SEQUENCE LISTING (145 sequences)

SEQ ID NO: 1 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 2 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 3 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 4 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 5 - DNA, 1000 bp, Arabidopsis thaliana
  misc_feature (1)..(1000): Ceres Promoter PT0613
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 6 - DNA, 351 bp, Arabidopsis thaliana
  misc_feature (1)..(351): Ceres Promoter PT0625
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 7 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 8 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 9 - DNA, 998 bp, Arabidopsis thaliana
  misc_feature (1)..(998): Ceres Promoter PT0660
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 10 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 11 - DNA, 999 bp, Arabidopsis thaliana
  misc_feature (1)..(999): Ceres Promoter PT0672
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 12 - DNA, 1000 bp, Arabidopsis thaliana
  misc_feature (1)..(1000): Ceres Promoter PT0676
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 13 - DNA, 998 bp, Arabidopsis thaliana
  misc_feature (1)..(998): Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 14 - DNA, 1000 bp, Arabidopsis thaliana
  misc_feature (1)..(1000): Ceres Promoter PT0683
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 15 - DNA, 1000 bp, Arabidopsis thaliana
  misc_feature (1)..(1000): Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 16 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 17 - DNA, 1000 bp, Arabidopsis thaliana
  misc_feature (1)..(1000): Ceres Promoter PT0708
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 18 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 19 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 20 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 21 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 22 - DNA, 1000 bp, Arabidopsis thaliana
  misc_feature (1)..(1000): Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 23 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 24 - DNA, 763 bp, Arabidopsis thaliana
  misc_feature (1)..(763): Ceres Promoter PT0837
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 25 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 26 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 27 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 28 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 29 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 30 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 31 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 32 - DNA, 999 bp, Arabidopsis thaliana
  misc_feature (1)..(999): Ceres Promoter YP0019
gatataagtagaatcattttttgccgccgtttctcgctaacacaccgaaaactgaatcaaatctcctagctcttctacgcaaaatcgagtgcatcgacaatggcggaacgtggtgtcgaacgtggtggagatcgcggcgatttcggacgtggattcggtggtcgcggcggtggaagaggtggtccgagaggtcgtggtcgccgtgcaggtcgtgctccagaggaggagaaatgggtgccagtgactaagcttggtcgtctcgtaaaggaaggtaagatcacaaagattgagcagatctacctccattctctcccagtcaaggagtaccagatcatagatttactcgtcggtccttcattgaaagacgaagtgatgaaaatcatgccggttcaaaaacaaaccagagccggtcagagaacgagattcaaggccttcatcgtcgtcggagatagtaacggtcacgtcggattaggagtcaaatgctccaaggaagttgcgacggcgatcagaggcgcgatcattctcgcgaaattgtctgtggttccgatacgaagaggttattggggtaacaagattggaaaaccacatacggttccgtgtaaggtaaccgggaaatgtggatctgttactgtacgtatggttccagctccgagaggttctggtattgtggcggctagagttcctaagaaggttcttcaattcgctggaattgatgatgtctttacttcttctagaggatccaccaaaactcttggaaacttcgtcaaggtatgtactttcacaatggctgttttggtttgatgaactctgaattaggcagtgaaaaagtaatcattaccagttaagtgaatttatattgaagattaggatttagctgattgtattggtttgagcatgtgagtttgtgtttaagattgcttgaattgaaatgctttaggttgtttgattacgctaaattctgactaatgtaattcaaattgttgttgtttttttttggtc

SEQ ID NO: 33 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 34 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 35 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 36 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 37 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 38 - DNA, 1024 bp, Arabidopsis thaliana
  misc_feature (1)..(1024): Ceres Promoter YP0092
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaat

SEQ ID NO: 39 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 40 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 41 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 42 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 43 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 44 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 45 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 46 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 47 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 48 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 49 - DNA, 1000 bp, Arabidopsis thaliana
  misc_feature (1)..(1000): Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 50 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 51 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 52 - DNA, 1004 bp, Arabidopsis thaliana
  misc_feature (1)..(1004): Ceres Promoter YP0128
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 53 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 54 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 55 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 56 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 57 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 58 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 59 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 60 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 61 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 62 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 63 - DNA, 999 bp, Arabidopsis thaliana
  misc_feature (1)..(999): Ceres Promoter YP0275
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 64 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 65 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 66 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 67 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 68 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 69 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 70 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 71 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 72 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 73 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 74 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 75 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 76 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 77 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 78 - DNA, 667 bp, Arabidopsis thaliana
  misc_feature (1)..(667): Ceres Promoter PD1367
acagttttcttttctcatcttacaacaagtttccaggaggatagagacataaacgaagctcggattgtatcgttctttttagcttttattcacatccgaaagtcctgtagtttagattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttctcctctttcacttcatcacttggcttgtgaattcattaattcctgcgaa

SEQ ID NO: 79 - DNA, 1000 bp, Arabidopsis thaliana
  misc_feature (1)..(1000): Ceres Promoter PT0565
caccaaatatagtgttatttcaatactaaaatggtgttatggttggagatgccctaaagataaacatgacgagacacgagatttattaatttcttgatcaaccataacttaataacttaatattaatttcacttaataatttccaattaagtgaatctttacttcaccaaaagttcctaacgaactcttattttctagcatcaatattaccatgaactagcatcaatactatcatgaaaaattcctacttcctatccaactcttaataacaatgctagtcttaacaatattcatcaaaaacttgatatagaccttctaacttagccacgactagtatcggtgaataccaaaattaatgtattcatgagaacttgagatttctctaatgtattcttgttactaaacaagtaacaacactcaagaaatatcatgatcaaatattttactcataaactccatatttcacattttgaaaattttaaacagcaaatcacattgaattttcgtggtaaaagtatttaaaattgaaaaatagcagctcctgatttcaatgtataaatttatctttatatggtttatgtctccaacttattttaaaaaagagagaaagagcacccaaaaggtgaccgtttgaaattcgaatttatttccgtttgaaattcgaattcaaaaaaagtaaaccgaaccgagtctcgttactgactgtcacacattgtttccctaaaagctaattaacccatacgtggcgtaatataacaggtcagtgatcaatactaaataacagacatacacctttaaaattcgtgcacgctccaaaacaaaatctacacttcaaaatcaacggtcacgatcattcctcaaatttcaaaaaattatttaacctcacttccttcgctttgtttttaaaacctctctctctttctctttctctttcgccattaaaactctgtttcctttttcagagattctcagagaagattcattttaccctaagaaaaaa

SEQ ID NO: 80 - DNA, 999 bp, Arabidopsis thaliana
  misc_feature (1)..(999): Ceres Promoter YP0015
ttgagccttattgttgttattgacttttagccaatagaaagagatggaaattcaataattatccacaaaattccaaatcattggtgtacaaaaagatctaaggctgttatattttcaaaaaagaaagaaaagaaatgcaacaaatatggattaaactgtggtttgtaaattgagctttgcatgaaaactttatcactatgatttcactactccatatttattgactaaagtggcactaatgaatttcttaatcatgaaatcttgtatcaaaaagtactaaaataaacatgacattggcaattaggaaaattctaaattagaaattagtaaaaatgaaaggtgaaagggaaagatgatgatatgaattggttggtgaccaggagaaatgtatcccgatttttgcagacactttcagtgtccccattcatataattatggcccacctcgttaagatttttcattcaccaccataacaagatctaagcttagatttcatgtaattaaacatataatatacttgccaatactatctaataaagtatacttaagcaaaaattattactctagtgtaaggcgatgaaatataagtttagttgaaaatttatgtcgatataacaaagtataatgaattaagaccttggttttcgattaacaaactaattaaacactagttttgcctaataaaaccgggaatcgtattcaaaaccgaacgacaaaacaagggacaagttgagagacaaaaccaaatcagcatctttcttccagaaatgtcatgaccacatgacgtcatcttgacccttcttcattgtgatatctgtggataaagcgcacgtgtttaattcacgaaccttcgtagtaacgaaaaatccacaactttcatattttttaattacccactaaactaaaacaaatttggaaaaacatgaaaaactttttctttttttccaggttcgtgaacctcgtaccctctatataaacctcttaaccaccttccacata

SEQ ID NO: 81 - DNA, 999 bp, Arabidopsis thaliana
  misc_feature (1)..(999): Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataggcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaacttatatgagtaatagaaacgatcctaatattaggaattttagagattttctctcatctgtttcttaacttttcaatatttttattttttaaaattgtatgagtttctactaagaaactactgctggagttggtcttagcttcccaatgcttctccacctatatatatgcatatctccttcttaaaac

SEQ ID NO: 82 - DNA, 999 bp, Arabidopsis thaliana
  misc_feature (1)..(999): Ceres Promoter YP0080
aagcggcaatttagtaagaagtactcaaagtatcatttaccaaaagtatatggttttgggaagagttgttagggatgtattctttctaaacagatgatatgacgatgttcttgaaaactaatgttaaagacggaatctctggcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaattacgaaagaaaacaggaaatcaaatcaaaagataaagagaaggtaaaaaaaggcaagaagcactaatgtttaatatttatagttttctccattaaagaaaaagcgatgatgtgtgttctcatcttttgtgaaagtatatatattgcttttgcttttctcaaaagcaaaagactcatccaacaagaacaaaaaaaaaaactaaagctcaatccaaaagacgaagaatgcattggatactacaacttctttttcacttttctttcaaatttacaattatgattttcacaatacagtttattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgtttttatttttctgatgttaca

SEQ ID NO: 83 - DNA, 999 bp, Arabidopsis thaliana
  misc_feature (1)..(999): Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 84 - DNA, 1000 bp, Arabidopsis thaliana
  misc_feature (1)..(1000): Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 85 - DNA, 999 bp, Arabidopsis thaliana
  misc_feature (1)..(999): Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaaacttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 86 - DNA, 252 bp, Arabidopsis thaliana
  misc_feature (1)..(252): Ceres Promoter PD0898
cgcaggcccgatcggcctaaataattatagtcataagaggacccaaataaataaataatgggattctctgaggtgtatttttttatgcacacgtaaaagcgtgaaagatttgtaaacctactttatatatatacttccatctctttgtctttgttacaatttgaatcagagagaaattaagaagcgaaaaacaaagaacgagaggaggcgagaggtatagaagaatattccttgtggccggcaaggccaatc

SEQ ID NO: 87 - DNA, 283 bp, Arabidopsis thaliana
  misc_feature (1)..(283): Ceres Promoter PD0901
caaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagctttttaaaatatttctccacgtgcctctttgtgtttgtctatata

SEQ ID NO: 88 - DNA, 2101 bp, Arabidopsis thaliana
  misc_feature (1)..(2101): Ceres Promoter At_pDME
gcataggtaccgtcgacgctagcacttttgtcagatttcggctctgggatttcatgtccagtagttgtaactatctgttcaaggaatccagtcttttcacctgttctgaacagactgggcgttgacggtacatattgaaacgaactttcacagactgacccaactgcttctcttctaattggtgaattcaaattgtaaagaggcttgttacacgctggaggtccatctgttcaaaaccatgactttcttaagatattctaattcatgttctactctaagtactgcaagaacggaagaaagggaaaaaaaaaaactgaaaatagatttgatcactcacattgacgaaccttgaataagctatccccaccactcagcaagcttgtgaaggagaagggcatctgatcaggattcaacaagtcatccaattcaaactcattatagccagcttgagaagtgccactctgggtgaaaggaccatttaccatacccacatctgaagtaaaatttccattgaccggctctacattaccggtggtgtttctgataactggtgctaaaggagaagtcacctcactcctgcccaaaagatctctctcactattactccaagcttgtgcatgtgatcccgctaagctattgatcatcatacttaagttggtaacgccctgatgagcaccatgatcaaaaaccccattatgatccacaccagtgttgcagaatcccctgtctacaaattcaccacctggaaacccatttagatcctggtttatcactctctcatctaccatcagacttgatctaggttttttgggtgtaaatggaatccaagaacccatgaactcttgttgagtttgattctccaaaggaactcgaaaatatctatcccccggatcagccctcgaattcataacacacttgatgaatcactcccccttcaaaccttatcactacctcaccccaaataacactcacctaactctccgaaaactctctccaaacaccaaaacaaagcaacatggacgaacactcctttttaatctgacttggttagagaaggcgacattaactaagaatctgcaccataaaatgcaataaatgattttttgtcagatatttttctcatcaacaaaacgagaaacacattgaatagcgaagaattttttagctggaaacggtaaaagaagagcgaataaagtaagcaatgtttatggggaaagtgaagataaaagcaaatcagaggtttttattacgcagaatcaaaatcaggtgttaatagctgagatttatgatcagaaaacaggaacatgtattgattaacataaaaacccagtgattcttctcgtgatcggaaaatgtaacagctggaaaatttagcagaacaaattccagataaaaaagggagagagaaaaaaaggaaataatatgtaaataaacagaaaatggagctaaaagctcaccgatctaagctttctgattctcaggcgagtcaatgccggagaacactgagtcagctatgccgttagagatagagatagagagagagtgtggtgagatgaagaaggaataaagtgaaatatcagttggcgttagcaatatgggcctgaggcccaaattatattcacgaggacgttgttgcttaagtgtattttgttgttaattgtcattagtcctattaaaatggtttagtttgcaagttttcagataaagcaacaacgctaaacccaacttttgttggttataagttataacttataaatgttcattagaatcttactaaatttaatgttttggtcttcttcctatatttagattttaaagtatgtcaacacgatttttaaatgaaatttaatgttgcaaacttgcttcttcatgccaacatttgaccgaattacataaaggaagttcaatgctaaaatcgtatttttgtaaaacgctcataatcactattttcgctagagtatctaataatttccatatattcatgaaattaatttgaaatttgacacaaaattctcgtatttttttaacgtaaataaaggtataacaccgaactcgtattttacgactatgttcgtttctaattgcaccatctagaaaagct

SEQ ID NO: 89 - DNA, 1725 bp, Oryza sativa
  misc_feature (1)..(1725): Ceres PROMOTER ID no. pOsFie2-1
ctggcgccgatctccatcctgctagcagaggtggcgccggcgtcctcctgctcccgccgcattcgctcaccggtgagcatcctaatccatcccatccggtctcggtctcggcctcggcctcgaatttcgtctcctcgcccgccgcttgcgccgatccccatcctgcgagcagaggtggcgcccggcgtcctccttcttccaccgcatcgtcgctcgccggtgagcatccctccctacccacacctcatcagttattctgcgttatccctcaaaaagtttgggggttcaaccgaaacagggttagatctccctctgcctagctccacctactttcaagttgttaattttgatttccgtttttagcagatgtaatttggttactgaatctcatccaaacacccttagaacgaaaaatgtaagctcacataatttgtactcatccaaactcacataaatttcttaagaagaatctagaccacaaactacccatagtcacacaaaatatctttttatcaaggtatattcttataagtaatttttattccacaaaattttctaaaggacatactgtgacacttggcattaatctaatttaactttttttctttatcaggttataaattttctaagacatttgtgccacttggcatgaatctaatttaacattctttctttatcaggttataaattgaaaccattgaatgtactttgtcttcccgtctctaaaacattgtgatatatctataatattttaatatttttaagtttaaaaataatttagactaataatatagatatttaaattaggtaattaataaattcaaatcaactatagtttagaataaaaaaacaaaataatatatgccaagtacaatctaataacaatagaatgttagaaacaatatgaattcaatatttttataaattttaagtacgattcatacataaaattaaagaaaaatataaatttatatataagtgagaacataataaaatgcaagttaaaattattaaaggaaaaatattattaataaacaatgatgttgatgtatttctaaatattttcacgtattatttattaagtgtcaatgattgaaatggatgatttaccatgtaacagattaattattaaaattatattgttttcatccgttacaatgcacgggcattttactagtctatataattgatcgctctatcaataaaccttaatatcgacaagtaaccacaacgttaagccattgatcgacaacctaaaagctacaactgaccataaatttcactggcttagatgagatgtaaacacataaataaaccataaaaaacgagaattcggagatgatggcaccccaagtccccaactccaaaccattttccggacaaaaaaaaaacattcaactctaaaccacagactcatgagaccatgtaactcaagcaaatctgactaacaaagataactcgaaatcaaatcgagaaataaatgcagatttgctaaaaagacgtgtgtgaaccggtcccaaattctcacaaatctgatccatgaaaatctgcagccgttatgaacttccaaatttggttccaaactctgaacttttgctcgccctactaacccccctcacaaatcgcccaaaatccgaacgacgccgccgccggaatccgccaccgccgccgacggaagcaagggcaggtcaggccgtcgtcgggagggggggagaggagaggcgc

SEQ ID NO: 90 - DNA, 3000 bp, Oryza sativa
  misc_feature (1)..(3000): Ceres PROMOTER ID no. pOsFie2-2
gcttaacacatgaactaccaaaatatactgatcactttgttctagtcatacataccttaagtcattttattctgcagtgtttggattggagggagcattctagcatcccttgggtcgttccagcaaatgtggttctccaaagcagagtaagcacaacacagtattttaggttatgtttcccctatctcgtcacggacagctcacaagttaatgtgatttatctcactatagatacgaagaacatggagtatcctacatccaaaggaagtgcccatgaagttgtggagcatcgctacgatttgtgaccaaatttgggtgcatgtgggcaatcgtattacagccaccctgttgttgatctatatcgactattatccgacgatatttatcattatattatgactagttagtttgtagattttgagagggcaacataagaagcaatccagcttaacctgttatgttcttgatggtagattctagttcatgtgttgaatctgttctccctgctgtagaatgtatcgagttgctgctctctactctgtacttttagaataccttttcaatcatttggagtcagctgattgttgtactacttatacgccacctgattagtcatgtcaacaattaaacttgagcactggttaagttaagagtggcctgattgtagttgataatcacattttattcgtagacattgtatgctggatctttatcagccaccgtcagatcatcctctgtaataaatcttcatcagacgtgtgtgccaatcgcaaggaacacgaaatgcatccgaaatgttactctgagttaatcaatactataattcttggtcaaattaattatttatatctataaagtttaaattaaatttaggaaaatgaattcatgcaaatcttgtggtaagttgtcaatttcataaaaaatccagcttactactccctttttaggagtgtgttgtggctgcacacttctgccttttgatatatacggttctattctcggtgtactcctttattattattaaaacaatcccagttacttggtaagtgctaatcacgaatcaaagtcaacataacaaatcatgtgcgtacagctataactcgattacacaaacaacaaaattcatatttgaacataaatccagttgtagcatatctggtagtataaagttttttttttgtatagaagagttttaatttctgtaagttttggaaagcatttaatcctagaaattgtagtgtagctcaactaaaaaataaatgaacttgaatcgaaattgggttgtatcataaatctttaccactcaaacgaatatttatcctaaaccacaaatgactcttttcatcaaggaatgttttgttttcagcattttaaaaaaaaacttttctaatatggttttcatgtttcgttcttttgaaatttaacatctatttaatttgcacggctccataaattcaacggatacatattctgaataattactaaggaggcatatatcggctctcttaatacaaccgcttgtttctcaaaatttattttgagttttgtctacacattctcaaggacggtacaaacacactatagatgttcacaattttttttttctaaagttgattgatggacaaatgtttgaacatataaacatataagcactgaatatttgcttatgcaggaggtatttatatcaagttcgatactttactaccatagtccctaggacactaaaatgccttcaatgatctgatgaagcctaagagagaatattgatcagtggagcgacttgcaactacacatggcacaagtagactagacacggtatatattcatattaacttgttaaaattttactacttaacagttcacttgtggtgcatccatatcaattcttacttacacaatatttgtaaaaacaacctaacactataggatgacctagacaacctttatgtcaatcacacttagaagatgatcgtctttttaataaataatgtgtactacacaccatgctctccatatagatcaagatctacaaacccttccacttataaaccttaccaccaaaaactcattaagttgcttcatttatctatgctattaagaaaaaaacttatttcgtttatgccatttctagaaatggctagtcacactattcacaatattatataataaataaaagtttcaaatattcatccaccaaaaatcatcaagtcgtgggacttatatgttaattagagaagtccctttgggtgcaatcgattttggaaaccctaaattttttctatacatagaagagagagatgtctagttgcaattgcttttgcgatgtgccaaccacccttctagctttcatccacgtctacttaattgccattcttcttcttctttttcttcactattactacctcctatcttagcgaatcttcttcttcttcactattactacctcccaccttagtgaattcatcctcattgttcacaatgacattgctaagttaactaggtatgctaagtacacaattagaatataacctagagcctttgtttccatcatacttaaaagatgacatttttatatagataaagtgtgctactcacaaggcttactatatatatgtatgatacacacaaactccacaacccaaaactctttcaagttgtgtggcccatctatgctattaaaaagcccatttagcccatccaacatgagaaaccctagggttttttccctataaaagatacctaggttattgttgcttttccaccccgcccgccgccgctccctattcctatttaatcccatctctcttcctcatcaccgctctcctctctccaggcaagaggtacgcactttttgtttcggatttgaaatctttgcttcgttttactatcattggtcataagttcttttttgaagatgtttgagaataagtttatcattgagattatcgtcacttgtgataggaagtacgcaacctcaagccggacaagacgtgagcaaag

SEQ ID NO: 91 - DNA, 2023 bp, Oryza sativa
  misc_feature (1)..(2023): Ceres PROMOTER ID no. pOsMea
gagagcagaacatagtagccgctgttttctgggggtgcaatttgtgcaagatcgctatccttatggaccatgcaagcaccaagcaatattaagccaggtccaacagcggtcttggggaattcagaaatgagcttaaaaacctccttgagctggccagctcagccaaggaggtccatcatgcatgtgcatgctcaatacttggaattattgcaaaatgatcggtcattgactggaagactttgcgcccttcctcagccaaccttatgtggctgcatgcatagagtaccaacaggaaggtagcgtttgttggaataaggtttgcatccagcatgtccttgtagagcttcaaagcctcagcaccttggcccatgaaggccatatccagctaattgcattccatgagaccacattcttgctatccatactgttgaagtgaagatgctccgagcttcggaaatgcttccacactatgcatacatgtcaatgagcactgtcatgacataaacattgggccccaagtcctcctcagcgataatcctatgcagccactttcccagggacaaagctccaagctgtgcacacgctgaaagagagctagaaatgatgattggatttggtcacacgctaagtaccagcatttgctcaaagagggcaattgccatctccgtccagccattctaggcataccctggtattattgctttccatgattccgattccgtggtcttctatggcatcgcattgaaggccttccttgcagactccatatcatttaacctacagtacaatatggtaattgctgtcgacactggagaattcgcagtaaatccagacttgagaggaccatgtaagcattgatcaagcagttcattcccaaacagactatacgggatcagtgccagtgctcgagtttggcttcaattccaaggccatcaacccaataaacagattaactgatgaaccaaccatgcaattcgccgagcaaacatagattaagcattgtaggcaaccaaatctggattctccatcaagtcaaagagacgccatgcagaattccacatccccgctgtatacaccgagatcaaccggtcagaacatgctcatactccgccaaccctctcttcagaacatgctcatactccgccaaccctctcttctctgcaagaggcatcctccccaattccccattgttatatctgttgctggtaagaccgttgccagcgtggttgtgtcagaccgaacagactctgcactcgccatcctcacgaacgactccagggcctccgaaccaggaagcccggccggccatcagcgtgttccacataacggtatccggcgactgcacagtgtcgaacaccttgcgtgcgtggtcacctctggacagcatgaagcgtacaggctacagcttggccaatgcggacgccacgaacgtgtcggcggcgtaacccgcgcgtgcagcgcgccgcgcgcgggctgcggagtcggttggagacgacacgccgccgccatgagagcaatgagcgaggtggcggcgaaggcgaaggagaagtagtcgaggcaagcggaagagaaggcggcagcggagaaagcgatcggggcggcggaggaggtgggtgggagggagggacgcgtagcggaggtcggaggaggagggagctgaggtttccggggcgggggtcgagagggtagtgtacggaggcgagggacacggcgaggatctggtcgaggtagcgcagtgtgaaggaaagcgcgatgaggcggagggcgccggcgaagagcggcgcggcggatagcgggaggaggcggcgccggcggggtctcatccgattggaaacagattgggaagggggagggggtaggaatacgtggcgtcggcagtattaggtagagagagaaaccctttccatcctttgtctcttagccccgaaggagagagaaaaatcagaaaaaaaaaaccctccgcgtgtgggggaagcagagctccggacgctggcgccgctcgcgccaccgcacccgcaccgcc

SEQ ID NO: 92 - DNA, 2034 bp, Oryza sativa
  misc_feature (1)..(2034): Ceres PROMOTER ID no. OsYp102
gaacgacccaaacgcgtaaatggtggtactggtttccctgctttgccgagtaccagcagccacgaagaacgttacacaatcgagtacaaaatctataagagcaagtttaatagcatagccaaatactacctctaaatcatctatagccaatttaatagttcatttattcaataattacttataaacatatactacaatcattaatatatggtcttacttcttatacacataatattttggagtccgtgttacagctggctataaatataagggattttggttggatgtggtacatcctattataatgaatctagacatgaaacctgtccaaattcatcgtgctaggatacgccacatctaaccaaaatctcttatctttagggatggagagagtaataattaaatgaagctaggtagagtttcccggtcaatacgcttgcgtgtgcttataagagcatggccaacagtttcccgatactcttcccaatatcagttttgaggagttttgttggaaaaaatcgctccaacagtagacctaaatcacccctaaaagcttggcgtttccaaacccgcatatttcgttctccacttgtagggaagagactcggcgcccaatccttcaaccgcatgcacttcgcgcgcgctgtgtgaaaattttcctaccaggttcttctttgtgcgttcgtctacctgtgagtcaatccatcacgccagcagcctcatcttccccgcagctgtctgggaaagcagccatggctcccccaagcttccccagcgtcgacatttttttctcagcggcagcgccagacccatctccaacccaattgggcggaccttcgtcggcgctcccccagcaccaccaccgactcgaatcggccgtcgcccctattcatctccaatcgtccctcgaccctaccgcatcctgcagcacagcctgtctctcgcgtcagactggcgctgcgctccccccggtaatgtgcaggcgacaaaggccccatgcgatgcgaccagcagccggcgacaaccggaggtgcccagtcgctggccttcatcgaatcatcgtgcacctcggtcggagtcgatttctgattgttgctgctgctcaaatctggagcttgctattgctgagaactgcttggtggtggtactggaaatttgttgtttgctggctgatgaaaactgttgttctttgctgctaaaaactgctgcttgctagtactgaaaagtactattgcagctgctgaaatatcttgctgcttgctgctgaaaacttcaagttgttaacaccgttcacactaaaaaagctgaaattttttttctgggctgaaaaccccattgttgatgattgcagaaccaatatttttccatgtaaaatacaggagatcgtggtaataatcaagtgaaatatcattttggggcaaatactcagatcgtacctgaagccaatggaaacattgttcaatgcttaaactgtcagttatgatgtcaaagagattgatcactgaatgtcctgaaaggagccgtgaggaggatgcagcattgcagcgtgcgcgagcgtgagtggaggagaggaatgacgattctgttggtagttgtcgatgtggcctactttttttgttttgaggattaaattttgggaatctcttggagataaaaggtattctcataccttaaatcctttttagagatctaaaaaaaatgatttaggggattgaattttgggtggctgttggtgatgctctaagttgcacatcctggggaaaaacctccctaatccatcagcaaaccgatcaaccacccacgacaagtcgacgccaccgttttttttttctccctcctaagtcctaaccccacaaaaatcccgcgaactttcgtctcaccacgcgccgcgtgccccctacaaataccaaacaacacccaccacgtccactcacaaaccacgcaggaaacctcagaaaatcaccgtacgcgacgcgggcccaagaaaaccccgacagaaaccgcgcagcagcaacaccaccaccggcgtcggag

SEQ ID NO: 93 - DNA, 1877 bp, Oryza sativa
  misc_feature (1)..(1877): Ceres PROMOTER ID no. OsYp285
ggcccgagttaaacgatcttccacgtgtcagcgaatcctagtcgttcgatgaatctgaatctgacttgtggtggttggacggccacgtgttaaaaaagggaaacgtccgcatcacccgatgctgggacatttgcaatttcgatccagctgtagattgaccagttgttactctcttttttttaacaccatacaaacgtaatactccctctgtcccaaaatataagtattttttttaacctcggttcagtcttcgaggtgctactttgaccaataatatttataaaaataagatgttttaaataaagagagttgcatattatgatagctcgtttaatgataaacaaagtaccatcaaatttacatgattaatctttttaatttatttgctattaatagttaaaatttaaaaagtttgacttcacactgttctaaaaatacttatattttgggacggagggagtacacattagagcaggtacaatagcagactagtagccagctataaacatattttaatgagataaaagatgagagagaacagcgggctacagatctgtagccagctgcagcacggactccaagacattgtgtgtgtatgacaggtgggaccatatattaatagtacagtaagtaactattgtatgaattggctattagattagctataggtgaattgtagctagtagtgggctatactattgaacttactcttatatctctcaatatctccagaaaactaggacgatatatattgatattaacaaagtcatcatagatatctcgctatcgacatatatattacctatcactgaaaaaataattaatcataaatgcaagcacatatactacgttcaacactgaatgtaggtagattggtagacgggttccaccgcaagaaaagcattgcaccagtgaagaaagaaacatcggaatttgtatgtagtttgttgtttgatgaattcttttgattaaaaaaaactaaaatcagagttgattcagttaatggtgttgcctacgatatacttccatatcatgatatcactgtagactatgaatcatatctttaattaaaactaaatcaagaaattaagtatgagacctcaactcaatgaagaatttctagttgaaaaacattcctagtgtgcgttcggatggaggtagggatcttctctccgttcatataaaaccggatggttcattagaacatgattaattaagcaacagttaatctaaaaataaattaatattttttaagaaatttttgtatagagatcttttgaaaaaaatacattggttagaaagcatactaataaaaagagaaaaataagaacatagtactatagtagaaaatgagaacttggagtatttgagaggatgggaaataagaagattaagaagatgcgtaaagtgaacggttaacgcatgattgattaattaaatattaattattttaaatttggaaaataaattagtatgatttttaagcaacatatatatatatatatatatatatatagaaaaacatagttttagaaaatataagcgtgtaaaacgatatgcaggaacgaaacgttgagcattcaaaatttcaaattgaacatatgaatcaagagagaataaaaaaagaggccttctaggctggcatggacaattggacatgttttcaactagggtttcaagcttcgagcatccacttttgtccttgcaaactttatacggcaaggcccgtgaatctagccccccacaccaccccacccgcccgcgccgcgcggccgcctcgcctcccctcccttctcctcctctccgcccccgccgccaggccgtccacctccgccgtctcctcccccattcgcacccaaggcgctggcgcggaaggc

SEQ ID NO: 94 - DNA, 1912 bp, Oryza sativa
  misc_feature (1)..(1912): Ceres PROMOTER ID no. p472e10
gcgtacatggaagttttatgagattgttttagcgttacattattgttctcatgggttttgttgaaccgtgctatagaaccagaacgaagcaatagtcacgtaggataaaccaaatcaccttatctattaggtgtatatggaagttttatgagattgttttagcgttacgttattgttctcatggtttttgctgaaccgtgttatagaagaacccggaacgaagcaatagtcacgtaggataaaccaaatcaccttatctattaggagtatatggaagttttatgagattgttttagcgttacattattgttctcatggtttttgctgaaccgtgttatagaacctagaacgaaacaatagtcacataggataaacaaaatcaccttatctattaggtgtatatggaagttttatgagattgttttagcgctacgttattgttctcatggtttttgttgaaccatgttatagagcccgaaacgaaacaatagtcacataggataaatccaaatcatcttatctattaggtgtatatggaagttttatgagactgttttagctttacgttattgttctcatagtttctgtagaaaccgtaacctgaaacaaagcaaatggttacataggacaaaccaaatcacacaaacttcactaattggtaagcttggtaggctcgcaggaacgaaaacacaactaattggtaaaataaatcgcatttgacatatctagctaatccgattaatcttatactctcatcatctaatttttagctgaccaccagcttccaaattttgaaatttgaagctttgattataggatttatttttcatctaagtttactttccggtcttcgatttcaaattgataatgatacaaatataaaaacttttacttttatttgaaagccaaatgaaaaataccctgaaacgaagaaaaagtcatttaagacaaacttagagataccccgatgtgtatgatcaaaatggggtctgatacactgctgatcagttcccacattgattttggtgtgatattccgttccataatcgtctttaaaaaacaaaagagggaaaaaaacaaaacactatgcaaccgtgcaaatgaaagcatcgtcaaatgattaaaaacgtcaaaccaattcaatcaaccccaaactccaaaccaactttttttttctcttttcttttttttctttttgtcgatcttgagcgaagcaatcctccaaagtccaaaccaccaatcgaagcaagaacacaaaaacaaaaaacagcaccagcgaattcggtgccgcccatcggttatggctctcgccccacacatcttgcgttccttctcgcagcaaacatttcccaaatctcaaaaaaaaaaagaaagaaaagaaaaaccaaaagaggaggatgataccgtgatgacaccatgcaaggcagttcgtcacatgatctggttcgctccaaaaagctgatagtaaaaatcatcccaaaatatctcctcggagaaaaattcttaccacaccgtccctctcctgttcatccctgttcgtggccgaatcttttgtttttaccgaggaatcttttgattagtggttgtagtgacatcatggacagaagaggaggttggtaattaggcggggtaaaaaaggaccgaggcgacgcgagagctcgtctcctccactcctcgtcctcgtcctcctcctcctcttcctccatttttttttcttttctttttatttgattacgccgtcgctgtcgagtagcgcgtcagctgcatccgcggttataagtagcggccaccacccaccacccccggcttcctctcccactgcgccctccgcgtgagcggcagcaagtgttcactgcgttcttcttctcgatttatctttcttggtttcttgatctgtagcttattagcggcc

SEQ ID NO: 95 - DNA, 1946 bp, Oryza sativa
  misc_feature (1)..(1946): Ceres PROMOTER ID no. p523d11
ccctgattcttctgatggaactaggggaggctgtgtggccatttttcccgttggagggtttcgtctagatctgtcgggtgtgggacatgcggattgcaggtgctgtcggttgtgttggcggcggcgggtcctgccgggatagttggccgccgacggccgcttggctgttgggttgcacggtgtgtgctggctggtagcgaggatggttttagggtgttgggcgaaagctctgtccgactcatagccggcctgacggcgatgaacgtccttggacatcatgcaatgcccctcctggaggcgtcgtcgcaagagcatctccagtagagaccctaaatacaattcctaaacagtttttaggtgctaaggacaaaaaataaactccagcaaaacccatactacaggtcctaaaataggaaggacctcaaatacccctccgcagtccctaggcctgggggctgtagaccgaggccctatcgccgtttttctacgcgggaggaaatttcctgacgtgtggtgtctgtcttccctcccgcggaatcgctgccacggcgccgatcttcgccagctcgctggttccgccgctcgtggccgacggtgcgaccatccagtacctccaccggccactgcttgtcgtccgcgtgcccgcttgcttgttttttcgtggtccttgatcagttcgcacactgatgcactatatggtagacaagaatgttctgaaattcatgaccatcagaaacatgttctaaacaatcctgctctcgattggtttatggctaactgtggttctaaacgatcatggcataaaaattattgttctgttcctttaaagtttgtggtgcttggtaggttgagacaattaggctgcttgcaattatgcagtagttccttcaaagattattctgcagtgttgttcttttgtgtcagttgtgagttgaagtttaacttcaaggttttttttttctaggaggatttaagctctttctgaagtttctcagatagattagattggaaaaggtatagagttaattttatctattgattatagttcttatttaattgaactacgtagtgtcttgaatacttgccggtaggatttcactcccatgtttgagaattttgaatttgaattatggtatttaaaattatggatttgaatacaattgaattctatacattagaaatattcgtatttgaattattactatgttaaactaggtgtaagcatagagtataatcagaaatacaagagaaaaagaaatgggggctaagaaatagggtctgctggtagagttggaggtaatttttgaattcttagaaaatagggacagccctcattcaacctttgaggactctaaaatagggactactgctggagatgctctaacaccctgttcccccttgctgctgggtgaaaaccctttccagtctcctgtttatgcgatggtggcgtcctttccgacgtcgtcaccttcttcaaggcatcgtttttggagaaaccctgcaaccagtccccctgctttcccatccttctcccctattccatcccctcctcctccccttttcttctgtcaagggctcctatgcttggaaactctcatgtatctcttctctgtaatatattcaggtggggaaatgttggatttttattgattggaatactgtattgggtcatctcggtgacaccaaagctgtactttggtggagtagcaatctttgcccttattgaccggataggattttggttaaatttatctacgtttttgtttgcggttcatcttttttcctaccagtcttatacaagatggtacagtttagcaactgattgttacattgcaatatataaatcgaagtgatagaagccacctcaagtaaatctaactattgttcataattcaaaggtcaagaccaatttctcagttcctgcgactgcgcgaaaaaacaaaacc

SEQ ID NO: 96 - DNA, 1951 bp, Oryza sativa
  misc_feature (1)..(1951): Ceres PROMOTER ID no. p524d05
cgagatccaccgatggtttacgcgtacgccgacggctcacacatcccccggtgcccaacagaaaccacacaccacccgcacgaaaaaaaccgaaccgcacgtgcgcgcgcgctccacgcacaccccaaacagacggcacggcgggagcgcgcgcgcgcacgcgagccgaggagaaaacaaacgggggaaacaagctggaaaagcaaaaggggaaaagaacggagcggaggcttcacccacggccaccgcgacgcgccaccagcgtgcggtgcaatgcaacgtacgccaagccgaaacggcaggcagcatcgcgcacgcacgcacacacaggccacagcacacgcgagcgacgtacgcgagtgcatgcagatgcatgcgcggggctcgcgcgagaccggccgatgggttcgcttctcttctctctcccgtcccgttgcgtcgtcatagacaaaagtcggttttgcttttggttttttggctctgaggcactgacgtgcgggccagcgtacgcctgcgtgccccgcatgtcatcgtcgacaccggccggggaccgggtaaaatgtgttgcgggagggagagggggagagagagatcgcgcgggcttcacgcaacggcgctacaaatagccacccacaccaccaccccctctctcaccattccttcagttctttgtctatctcaagacacaaataactgcagtctctctctctctctctctctctctctctctctctctgcttcacttctctgcttgtgttgttctgttgttcatcaggaagaacatctgcaagttatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtgcagagatcttccacagcaacagctagacaaccacc

SEQ ID NO: 97 - DNA, 1836 bp, Oryza sativa
  misc_feature (1)..(1836): Ceres PROMOTER ID no. p530c10
gcctctcgaccacgagtttagcacttgtgcaacatatatgcgtgcgatgaacatctactgatgcgccatgcgaattttagcgttcgttcatgacgcttccaacggcacagaggctgagcagcagcatgcatgcatggctcttgtgaaaacaaaaaaggttactggtaaatgacatgctgctgtagctagctagcagaatgcaaggcccatgcatatgcaatgctatgcgacaagtacagtaccagcatgtatggtagccagctaactaatctatcagcagaggcagcaagctcgtgcatggtgtgatgcacttctctccagtaatctagtggtaattttcacccaaagcgttgctcatatggacagtaattagtaatattaccaaggttcacaatcccgttacctgaccaaatactactcacgaatggtatctctggttttcgttaaaaccgttggtaaaccagcaaaaatagacaaaatttgtcaaaattttaaattttagttttttttttttaacttagccgggaaaccttgaagtttgtgctgtcgagctgtcctgggaaggacggttttggttgggattgtgaaccctggttactgcacttcatttttgaacagatattagtgcaacagacaaatgccaacgcatttttttctgtttaccggcaagctgaagcttttacgatccccatacagccgttgctgcaaacctgccaagaaagagcagcagaaacaggtgtcattttgtggtggaaagccaagtaaagtaaacagaagatggaagatagtgaggaccagggagtgaggcaggggacacatggcccacgcctccctgcacattttcgtgtataaatacaggtggatgcatcgctctcccagcatccatcggttctctgctctgttcatccatagagtttcctcctcttctcctttagtgcaaggtagagaagagcatgtgtgtgtgtgtgtgtgtgtgaactgtgaagtgcagagtgcttctgtagttctgtgttatgtccatagtgatcttgttaggattgttgctatggatgcatgatgttatggttgatctctgaattacagtagggacttttctgagatctctggattagtggggggtgctaaatttttttctggttgcatcagcttgggtttctggtattggtgtgggttcttgctctgaattttggttcagaatgtcgatttgtttgtgtttgttctctgaagttgagagtagctatgatccatccagcacagaactgcaggtcctgcctgccggctgcatatacaggacatgccattttgcaagctctgggcttatggtttctcttttggagttcttcttcttgcatgatctgtgttctctaacaaaggaagcaagatttagcaactttattcagagacaagaaaaggatctggcaaccttttgtttctgttttatcctactcgtaaagattgttatttaagcaaaaatttcccaaaagttttaaatataatttccatgatgtgccactctcatgtccttgaacctggcactcattatgggctcctcagaagtgctgtagctaatgtcactaatcttttgtatctttgttcatagtcttgtattttatgatgcttatccctttgtgctttccatgtttgatgtccaaatgtcatggcaatgtttttgacttctagtaggggttttagtacctttttgttagataagtacatccaaattctgtttatttattcaaaaatcattctgtttattcactgaaaacatttgtccattcaatggactcataaactgtctgtgtttttcaggcttgaggatccatctagaagatagca

SEQ ID NO: 98 - DNA, 1895 bp, Oryza sativa
  misc_feature (1)..(1895): Ceres PROMOTER ID no. y678g10
acaagcctatttcacccttacaacaattcggaagaatatagatgggttttaaacatttgataatatttgctccccactcagatttggttactcgaaattgtacaagacctgacattcgtcatctggacactctagtagataaacgtgctggctgatgctagataaacagatgtaaagatgaccacttcaccatcaaccgtaaaaccggacgaagatcaccaaaaattgatactttggagcaacgataggcagcttcgattcagatagcacaatacttaaaagaccacatactagcatcgaattgatacatctcccctccaaatgaggctccaaaaactatccattgtttgatacagcaagcaataggatgtgagaaactgagattggcatttgtatttcactactctcatctgatgagacatgactaggctgtaactgaagctgaatctaaagaggaagattagtgtgggattgcagacaaaactgctactacttccttcctgcactgcaagaagaagaaatctgtatccagtctgtgttgaaccccattaaagcacacacagcagcttcgattcagacagcaaaagaaacattctgatagatagcatcaaattgatactagtatttcgtttgtgtcaaaaaaactctcgatatgtcgtaatcaaagctcgaaaatcccatttgtttgatacagcagcaacagcaagaaaggaacccctactccgatccagccactgaaacagtactaatgaatccggattcgcgcattcatcctatctgatgtgatgaaaagaagctagagtataagaatctaatctgggagaaggttgaggtcagtcgtcgaaggcggatgaggggtcggcgaggtgggcgaagcgggcggcggaggcggaggagaggaggaggaacttgcggacgcaggacctgacgcagtcctcctccttcttgccgagggttcgccggtagaaggtggtgacgcagtcggagaagcaccggtgcgacacccagttgtacagccgtatcctgcgtgcacaaaaatccatccatcgctactccactctctctgcgaggaggaagggaaggaaagtaagagattaaacgtacgcgtctcgggtctggagcttgtcggcgacggcctccatgcgcgccttgtcctcctcctcctccccgccgccggcggccatggcggcggcggcgtccatgctcttcttcagtagcagcacaagaagaagaagaagaaggagaaggagaaggagaagcgtagcccaagccctaaggccctttagtatagttgaagtggtgagatgggccgtggtgggccttcggtaattgagcccatgggctcaaccccgaaaatgccagtgggctaggtgaggtaaaccgtgcacgtgacgctttcagtttcttttcttttctttccttattatatcatcaaaaaaagaaaagaaaaagagaaaaaaaggtatggaagatactgtatagtatacgctagcagcataagctccgtccgtataattatttcttgtacgcatatgatgtacagtatgtattttacgagctgtatactaccattgcgttggatttatgctggagctatttgcctatgtagtggagtattctagaaggatgcttgtgcgccgtccattgcctgcagaaacggacggcgcgggtgggtgggccccacagggcggtgactgacgcgtgggccaccacattgggatttggctttgctttgctttcgtgccttgtcagccgctgcccccggccccttcttctccttcttcttcttcttcttcttcttctccctcaccatcaccaacaagagagaggaggagtggattcatcgatcgagaagtcgaggtagtacatacgttggattggattggaggaggaga

SEQ ID NO: 99 - DNA, 1971 bp, Oryza sativa
  misc_feature (1)..(1971): Ceres PROMOTER ID no. y730e07
tcagggaggtatgtggttatcttgccttcaagttttacattttgtttccatgatattcacatgctgtattgcaggttattgctctttgtgatcatccatgcttgttggaaaaggaggaaaccaaatcattgttcaggtgaatatcggcacctttatttcatcagcatcaaacagatatgcagagaacttaaatggagatatctagtgcaaacactcacattcctttagtttgcttaccatatacttcatccttttgtttctctctactgattgagttttgactagaaatattacatgttagttgagcataggagtttcaaaaaccaaaatcttattgagaaattttcaaggtggtttatccctagttaaaagggctaggactaaatcgattaactatgcaactggcatatcaccctaacttaatttctaaaagagttctgctcatgaacttccataaatagttgactatcatactgaaatttgaaattctagtgagtatctgatgccccatctttgctgcagtgctgatgccatttctcgagcaacaacaattcttgcctcgattcctggaagagcaactggagcatacagccacagccaggtgagcagctcaggctgatacatttactcactacaaagaaaaaaaaagaatcttaatttcaccgtactcatttttcctagggcatcaaagggctgcgtgatgcaattgctgctggaattgcatcacgtgacggataccctgcaaatgcagacgacattttccttactgacggagcaagccctggagtaggaactttaccttctttttaaatcttactggacattttttgaataaacaggaagcagttcgaatctcattatgatgctattctccccctctgttttaggttcacatgatgatgcagttactgataaggaacgagaaagatggcattctctgcccaattcctcaatatcctttgtactcagcctccattgctcttcatggtggagctcttgtatgttttgaattctcagcacattttcaatatggctgcattcatgctgcaccaaagcctaattgagagcattttgttttaggtcccgtattatcttaatgaatcaacaggctggggtttggagatctctgaccttaagaagcaactcgaagattctcggttgaaaggcattgatgttagggctttggtagttatcaatccaggaaatccaactgggcaggtttgcattcattgctttcttgtctaatttggagagcatcttggattgttgcaatttctgttcacaccatattctgcatgtatctacctaaggcatatatatttgcaattcttgtatctttttatgtgattttccattgttagggaacatatgtatttttgtttgtctgcaatgtgcatgaagcatttgcagctggtgcaggtacccaacaaaagaactgtaatcatgttttaattcatttgcaggttcttgctgaggaaaaccaacgggacatagtgaagttctgcaaaaatgagggacttgttcttctggctgatgaggtaagcgattgttacttgagcaactccacaacaaactttcagctgcttaattccttttcgctgtgctgtctgtaacatcaacactattcatattgataggtgtaccaagagaacatctatgttgacaacaagaaatttaactctttcaagaagatagcgagatccatgggatacaacgaggatgatctccctttagtatcatttcaatctgtttctaagggtaaatacgatgatctgttttcttattttctattggcactggattctcaaaaggattttcttgctgacaacaggatattatggtgaatgtggcaaaagaggaggctacatggagattactggcttcagtgctccagttagagagcagatctacaaagtggcgtcagtgaacttatgttccaatatcactggccagatccttgccagcctcgtc

SEQ ID NO: 100 - DNA, 1773 bp, Oryza sativa
  misc_feature (1)..(1773): Ceres PROMOTER ID no. p756a09
tgtgcctgtgccgctttacagccaagcccattagcaaggctcaaagatgggctagtttttctcggcccaagccgtctgttgaacagcgtggagaaggccacacggcccacgtgcatacgcaggccgcgcactggatttcaagatgggctgcgcgaggtggacggcccagattgctacggccttctacggcgtcacgttttttcgtggtgcggctggtgcccgtgcttcgcgtacacgacagtgtacacgctgcactgcactccaaagaaatccgccgaaagtgcagttatacgtagcgacaatctgcaatacgtaccaacagccgaaagcatatatggacaagcagccacgcaagccatcagcacaacccacacgaagagcaggttttttttttcgaatcaagccatacggtagtgcgacgtttctattgatatagcaggaaaaaaaatacaaatctatagcattgagagactagttaggagaagaaaaagacggccacaccacatgcctacatctgatcctgctactgaaaacaaaacaagcacacgacacctagaaggaatggttcacactaagagaagttttaacaaaggagagaggtggttgttggaatcaacatgtaattccaatagaaaaaagaacttgattagttgtagtaatccgtaagtaaacagaatcatatagataatggtacaagcctgacccagttgttgatattttttttaatctccctgtcttgcacgtgcggtatagatgctaatgtgatgtggcagcaccgacgtcacacctgtgacatctggccatatgtctacagctaatgctgtgttttgttcaatttttattaaaggcaaataaatatctatatctacggttgtgcctataccaattgaagttatgtcatatgaggcgttttcgtgctatctactgatgaaatttacctctcgtacatcagaaccgtgcaatatcattacttatgtcagtgtaacgggataaattggtagagtttttgagagtggaagcttcctgttttttcaaaatttggtaagatagcaataacaataatgagtttggtttgttgtcctattaaaatttggtaatgccaaaatttagtagggttaaaaataacaacaaagtaaatattccttagtttaaattgttttagttgaaggttaaacattaccaaaaattggtaggttaaaaatgttaataaaaaaagcaaagcccttagtttaaattgtttcagttgaatgttcaacattgctcacaaaatgttctcttaaatagtactttattattacaaagagcatctgaatctgtattaaaaaagtacaaaaaaaaacattctgaatctagaaagggaaaatatctagaagcgactgcacgcggcccccacgaaaagcccatgcacgtgggccccatcccgaaaaaagagcaacagcctcaccgcctacctgcatgtgcaagtggacggtgcgcggctgcgcgccgcaacgcgacgcccccccccccccccaccccaccacccaccggccccacacgtcagctatacagtgggacccacccctccggccccacatgtcagcaagacagtgatacctcctcccccgcctcctcgcgcggcgcgcaacgcacacgcttccccttcatctcagtcgcgcggactcctcagtcctcacactccccacgaactcgaatccccaactataaataatccaccggaaaattcacaattcgatcgcctctctcgatcggagatttcgcaatttctccgcc

SEQ ID NO: 101 - DNA, 1993 bp, Oryza sativa
  misc_feature (1)..(1993): Ceres PROMOTER ID no. y760g09
gcttggaacagcagagatttggcataagaacaaatttgtaaatgtaatttgtatgatattgtagctagactgtttggagcaaatcaattccgtggcgctacaaaagaatctctttttgaaaaaactaaaattacaacaaaaacggcacgctttgcaaaccatggtgtaacgtttgcccacaacaacctgtataagaaaacaagctttacagcttcgtacaactctggttagcaaactaattttgtcacgctaaggaatcagtttctcatagcaccgaccagtttcacctataaattagaggatactgcacagcccttgatcacaatacagtgcatttctacaatcttttgttgcccattcatctgggttttcttctgcttctttttttttcctagagagtacggttttctttgtaattctttaatttgttgcaaccatgaatgtattggcatctaagatcttcccttcccgctccaatgttgccagcgagcaacaacaatcgaagcgcgagaaagcaactattgatgacgctaagaactcgtccaagaacaaaaatcttgaccgcagtgtcgatgaggtaaccgatcttccccacaaaacatattcataaataccattacttgattttttttatggaattccttattcatgtagaacatattttctatttgatgaattctccatgcatgatgtttcaatcttcttttttttattgtgtggagtatataaaagtaattagaatttgtagcacctggacatatgcagcaaattattcatctactactatagttcggatttatttttatcgatgcaaattggatttggatagaaatgtacattcttttattttagtcagaataaaagtttcttctatctagaatatactataataacatatctatctaaaacaaatatggtacaacacacttgcaactagcagcaagttccctgaaagatgtttgtctaatgctatggtgatctctttcactacagtttggtgtatgtgtgtccatagtagaatatgagtcctgcaaaagcaaacatcatcatgccaacaaaaatggcccatgtgccatcaataattcaaggtgcccgttgatgagtaacagaacatttgattgtgtcaccctaccacaaacacacatggaaggccattgcattccctataaggacatcatggtcattccaaaatgtactgacacctgctcaatgcagacaaaaaccccttcaaaaaacagaagaatctccctcttaaaaaaactgattaaatgattatttctgaaataaaaatgttgagtttttatttttaaatagtttatatcattctattcttttagaaacgtagtacaaacatagatacttacagcgtgcgcatactcatctatataaatgcacacctctgaaaaactaaagagaagtggaaaaaatggcaagatttactaataattagattatagtttttcacatctaataggaaaattatagattaaataattttttgaaagaaaaaaatatttgaaaacttatttattttcaagtatttgaaattatttaaataaagagtaaattttagaaaactacaactacagtgaaaaaactatcagtttgctataacttttacgtgatatgttgctacagttgtcacctacatgtcctgtagcagtatatcacatcaaagttgtagttttgtgataatttttcatgctattggtgcaaaaaactgaaatagatcattaatattacagcaaactgatagttctatcactgtagttatagttttctgaaatttaagatctaaaagaagaaaaaaagggggggggggggggtgagatttacacacagccacacgacacgaggcagggctaccccactagacaatctgtccactcaccactggcctcacttccttgatctcttctcgtcttctccaccccgcacgcggccacccccgcagggaccccgtgacccgcgcccgcgcccgcgcctcaccgca

SEQ ID NO: 102 - DNA, 1854 bp, Oryza sativa
  misc_feature (1)..(1854): Ceres PROMOTER ID no. p780a10
gggttacgaaccgggactacaaagggtttctccatcagtgcacactctaaagaaaatctagcaccaacccaggttagccgctatacatgaccggacgtcaccaaccctatggaaggatatgatgctgttaggtacatggattagttgctgtctagattacgtgcaggtaattaacacatccaggagaaaacactggacagtgcgtacgtacttaattagtgatcaaccaaaaatatgcatgatattgcaatccagctaattacgttaggtgcacataagccagatgtagtataagctaagccagccgtttccatacgacatatgcataaggatgcaattatcctgatgcacgcttgatttaatttgatgggatgcgtacatattttgattccttgtcctaaagtatgcaaaaatccctgtccatcaggtgtgttgtctacacacggctatgtctcattgtgttatatatgttgacttgaactttttcgcaaaatggatttcattaattggttccttttcaaagtgactttagtatattataggaaacggtgaagatgacctctataccacctaatttaatcgaccttgtgttgttaggtggcacatcaaatatcattatctatatctctacctataccttatataagtaacccaggggaaaaaaatcgaacccatgaattgtgagatcacaattcagagattaaaacaaggtatgccaaatatgagtatatagtataccatataaaataactcaaattcgaattaagaataaacatgaaaaatagcaattggctttgaagattaattacgtactctgctgaaaaaaaaaccaaaagaatctggaaagaacataagtgtgaaatttcagtatcttctcaacagtacagaagaattatttatattaaaaattgcatcatttttttggaaaagggatatatatatatacacacacacacaaacacacacacacacacacacacacacattcagacagaacataaccatatagccatgcacccgaccgatgctaacggctcacactcgccaaagtatggctagctaaattttgatcccatgaattttctatactctagcaggcctatcttcagccaacatctttttaatttcttccctaaccagaaattggtcatctaaggagtcaatttttattttctctaagttcaaacaaacttattttttttggggcgaatgtacatctaacaggacccacaggtagacgtgattttttctaaaaaaagatgttataaaattgcaccttgtatcaaaatactttgacatatatacattccaaagggagaatatgttgctagacacttgtaataattgattggttcagaaattaatcactaattgtccgtaaagggtttaattaatcgttagtggttacagttggatgatatatgccaaaatgaacggtgaatttcgaatctttcttgcatctggtggctattaattactttaggagtaaatttaaaaaactatatgtatgttaatatcaaactatcacaaactacttatttgagacattgtattataaactatagatttcgcaccaaaaatatcacaaaactacatatttaaagcccaaactcaaaaaactatggttttgttatataaacgttatatgtaaatatgtcaaccaaacgtcgtcacatggagaaaccagataaaacagactgacagtctggagaaccattaaaatcttacaagatcacacactgcaaactgcatgctctctctccctctcaacgcctatataagcacatccatcccccctatgatcaaagcatcacagaaaccataaacacacaggcatctgattagagaaatcta

SEQ ID NO: 103 - DNA, 1894 bp, Oryza sativa
  misc_feature (1)..(1894): Ceres PROMOTER ID no. y790g04
tccgcttgcttggagaattttgcgcgttcacaccggcagaactattatttttagcttaatcaaaccggccatgtgatccctgattattttctgtttttttaactcaccaaatttatttcaaattagaaacatattacatggttaaccttacatttgaatgaactaaagcaatcttcaaatctttcgcaaagcatcttttactaggataggctaggtgagatatgttgtgacaaacgtgagctggatcgatgctatagtttgtacacacctttctcatataaagagtgataaaactccaaggaaaaacagattagcacttttttggggccatcctaatgcaagcaagcaaggcttatatggcctgtgcttttttgctttaataagccttttagtccccttccctagtctcatgaagttcatggcaccaaacacctcaacaagtggcaaatgatgaaatgatgtaaatgcacaactactttattttgggctggacgtgttggttctcaactgaacctgcaccgctatcagacagtgtacataacgcaatcgctgagcaaaggaaacagaaaggctactgcccagcgccattttatttggccatttctgctgcaaaagctctctttatttgtttctgaatatttgaatgccaatttggcgacaccaatttctagagagtttccgtggtggcaagacaacctggtacttattgtatagtgctttccttttcgagttgattttccatttgcatttgcaaagatttatataacaaatttgagtataaagaatacatcagtgatgaagtggcgtgactggctcaaatcgagctaagagagatcactcgagcaataatgaacagtgaatcagaataatggatacgttactgtccagtacattgctactgatccttgatgcgtgtgttttgtggtgataagtttgagccgtaaaagcagtggtcgaagctaaacaaaacaacaccatcaaaccaattttggagttttatctgggatattatgcgtggtagtggtattcttggatgcctttggtgacataatttgttgttgaccccaactttttttaaggacaaaaatgtttgtgtcaacactagtgttactatgtgcccatgtcatatgtacactgcttaagcggtgagcaccagaaacatacaaccgatgaagcgtacgttgctcacacgagcaaaagtaactttggtgtaaagatatttggctcttctctagtttgttggagcacattacgttgcattttcgacctattataagtcacactaaccattttacattttcatgatctgctcaatttcgtgcacacctcctgtacatgttaatttctctctagtgctaattaacgatgggctctgcacaaactcccctggttttgatacagacaagtccaattttattcccgcttaaaactaacaaagcttgcattttatctataacacgtctaatttcttgtgggcactgcacatattcccctggttttgatacaggcgtatccaaaattcactcacacttaaaagctcaaaaaagctcccattttaatcaccacacgtctaacaaatttcttgttcacatccacagaagaagctatccatgctgtactttacattgcagtattagactttttatactacttttacattacattattagacctttttttaacacaaaaatccacctacccaaccaattttttgccgggctggtcctcctccccccgcatgagccgcccgtgcgatgacgtctcccggtgggtcacaccgtcacacaccgtgctataaataggggggcttggcctctccgccatgagcaccacacttcaccagcttcgctttgcacaaagcctcagtgcctcactgcacttgcaccggtcacta

SEQ ID NO: 104 - DNA, 8812 bp, Artificial Sequence
  Vector Construct containing a MET1 RNAi sequence
aaatccaagctcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgagcgctagcgcctatatcgctagcgatcgcgagctacgtacacatcatgcatcgcgatcgagcttcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctagatttcacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcctagaattaattcaggtaggtcagatttgagtaacaggtctaacaggtctaggaggagcaggaagctcgaaatctctttgccagaatccaacatcatgccatcctccatgcttgtatccagcagctctaagagttcctctagcagtgtatccaagagcctcatgaagtctaacagaaggatcgttaggaagtccaataacagcaacaacagacttgaatccttgagcctccatagacttaagaagatgagtgtaaagagtagatccaagtccaagtctttgatgtctatgagaaacgtaaacagtagactcaacagtccaatcgtaagcgtttctagccttccaaggtccagcgtaagcaattccagcaacaactccctcaacctcagcaacaagccaagggtatctatcttgaagtctctcaagatcatcgatccactcttgaggagtttgaggctcagttctgaagttaacagtagaagtctcaatgtaatggttaacaatatcacaaacagcagccatatcagcagcagtagcaggtctaatctcaacaggtcttctctcaggagacattttgtttagctgtcaaaacaaaaacaaaaatcgaaacatcagaatcaacaaaaatacatcaaccatcaactatacaacaaccaaaacgtcaacaatataatcaaacacagatccactgaaacaaaaccacatatcaccagttgagctatcatatcaaaccacgagacaacaggtatatcaaatctaaggaacatcaccaaccaaatacatcagaatcaactataaccagagcagatacagatcgacatgataaaaaacatgcgaagacgatatcaaaactaaacgctatcaattaatcagaggattatacatcagactcaataggaacaatattgatcgacgagtaaacggatctaaagctagagaatcaaaagcagtataacaacagcaaagaataagcgataatcacagtcaatatagagctaaaactaagaatctaaaccctaaacagctacaataatcataagaagatgaagatcggagacactaaagagagaaaatatctaacctgcaagtaagaatctgaaaggagtcttgcggctacgaaaatgtgagaaatatgagagcgcaccctaatcctggtcgactcgagggtacttatagctacgaggtgtctagggttttcgctttctctttgtggttctacttttactaatttgcccttacgcgttttgggcctttctatttttttggttgtgaatttacccaacaaagaattacaaaaatggatccacaaaattctcatacatttttttcttcaatttgaaatgttaaatagcttataattatgtgttgtttggttaagaaattgtataattgtataaatttttttataaaaaaactctcttgatgatcgaaaaggtgacggaaaaccctagccgtcatgagttggctttgatagatctatggaattaaattaatactagtatataaattgataaatcgaaattacagcctaattaatgggacataaaacatatatttatctggcgccagaattcgaagctaaatgccatggatgtttaaacctaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcagcccttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacagggcgctcttccgctcgccctttggcgcgccggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccgggcgcgcctgaagtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatggggggcgcacctattgacatttgaggggctgtccacaggctgaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaaaagggcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctattccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttagggctaccatggaggcggcggccaatcttgcttgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagtgggccctaagcttcatttaaatccactgcagtggttccaagaaagagagcaatggtgccactgaacctggtaatgagcctgttgccagcaagagaccgaagagagcagctgcctgttctaacttcaaagagaagtcattggacttatcagaaaaagattcaattatcacaatcaaggaaagtcgggttgaagagaaggaaatagaggctgttaatttgacaaggacgggacctgaagatggtcaaccttgcagaaaaatcatcgatttcatcttacatgatggagatggtaatctgcaaccctttgaaatgtctgaagttgatgacattttcataacagctcttatcatgcccttggatgatgatctggaaaaggataggggaaagggaatatgttgttcggggtttggacgaattgaaaactgggcgatttctggctatgatgaaggtgctgcagtaatttgggtctcaacagaaacatcagattacaaatgtgtgaagccagcaagcagttacagatcttattttgaacactttagtgagaaggcacgtgtctgtgttgaagtctataagaagttagctagatcagttggtggaaatcctcaggtggacttagaagaattaattgctggtgttgtccgttccatccattgcactggtctagaccc

SEQ ID NO: 105 - DNA, 612 bp, Oryza sativa
  misc_feature (1)..(612): N-terminal domain of OsMet1
ttccaagaaagagagcaatggtgccactgaacctggtaatgagcctgttgccagcaagagaccgaagagagcagctgcctgttctaacttcaaagagaagtcattggacttatcagaaaaagattcaattatcacaatcaaggaaagtcgggttgaagagaaggaaatagaggctgttaatttgacaaggacgggacctgaagatggtcaaccttgcagaaaaatcatcgatttcatcttacatgatggagatggtaatctgcaaccctttgaaatgtctgaagttgatgacattttcataacagctcttatcatgcccttggatgatgatctggaaaaggataggggaaagggaatatgttgttcggggtttggacgaattgaaaactgggcgatttctggctatgatgaaggtgctgcagtaatttgggtctcaacagaaacatcagattacaaatgtgtgaagccagcaagcagttacagatcttattttgaacactttagtgagaaggcacgtgtctgtgttgaagtctataagaagttagctagatcagttggtggaaatcctcaggtggacttagaagaattaattgctggtgttgtccgttccat

SEQ ID NO: 106 - DNA, 8862 bp, Artificial Sequence
  Vector Construct containing a MET1 RNAi sequence
aaatccaagctcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgagcgctagcgcctatatcgctagcgatcgcgagctacgtacacatcatgcatcgcgatcgagcttcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctagatttcacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgaacgttattagttcgccgctcggtgtgtcgtagatactagcccctggggccttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcctagaattaattcaggtaggtcagatttgagtaacaggtctaacaggtctaggaggagcaggaagctcgaaatctctttgccagaatccaacatcatgccatcctccatgcttgtatccagcagctctaagagttcctctagcagtgtatccaagagcctcatgaagtctaacagaaggatcgttaggaagtccaataacagcaacaacagacttgaatccttgagcctccatagacttaagaagatgagtgtaaagagtagatccaagtccaagtctttgatgtctatgagaaacgtaaacagtagactcaacagtccaatcgtaagcgtttctagccttccaaggtccagcgtaagcaattccagcaacaactccctcaacctcagcaacaagccaagggtatctatcttgaagtctctcaagatcatcgatccactcttgaggagtttgaggctcagttctgaagttaacagtagaagtctcaatgtaatggttaacaatatcacaaacagcagccatatcagcagcagtagcaggtctaatctcaacaggtcttctctcaggagacattttgtttagctgtcaaaacaaaaacaaaaatcgaaacatcagaatcaacaaaaatacatcaaccatcaactatacaacaaccaaaacgtcaacaatataatcaaacacagatccactgaaacaaaaccacatatcaccagttgagctatcatatcaaaccacgagacaacaggtatatcaaatctaaggaacatcaccaaccaaatacatcagaatcaactataaccagagcagatacagatcgacatgataaaaaacatgcgaagacgatatcaaaactaaacgctatcaattaatcagaggattatacatcagactcaataggaacaatattgatcgacgagtaaacggatctaaagctagagaatcaaaagcagtataacaacagcaaagaataagcgataatcacagtcaatatagagctaaaactaagaatctaaaccctaaacagctacaataatcataagaagatgaagatcggagacactaaagagagaaaatatctaacctgcaagtaagaatctgaaaggagtcttgcggctacgaaaatgtgagaaatatgagagcgcaccctaatcctggtcgactcgagggtacttatagctacgaggtgtctagggttttcgctttctctttgtggttctacttttactaatttgcccttacgcgttttgggcctttctatttttttggttgtgaatttacccaacaaagaattacaaaaatggatccacaaaattctcatacatttttttcttcaatttgaaatgttaaatagcttataattatgtgttgtttggttaagaaattgtataattgtataaatttttttataaaaaaactctcttgatgatcgaaaaggtgacggaaaaccctagccgtcatgagttggctttgatagatctatggaattaaattaatactagtatataaattgataaatcgaaattacagcctaattaatgggacataaaacatatatttatctggcgccagaattcgaagctaaatgccatggatgtttaaacctaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcagcccttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacagggcgctcttccgctcgccctttggcgcgccggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccgggcgcgcctgaagtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatggggggcgcacctattgacatttgaggggctgtccacaggctgaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaaaagggcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctattccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttagggctaccatggaggcggcggccaatcttgcttgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctctaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagtgggccctaagcttcatttaaatccactgcagtggttgagctaggtggttcagacaaaccaaaggatgggcaatcagagaactgtcttgcaacacttgacatttttgctggttgtggaggtttatctgaaggattgcagcgatcaggattgtcacttactaaatgggctattgaatatgaagaacctgctggggatgcatttggtgaaaaccatccagaagctgcagtatttgtcgaaaactgcaatgtgattctgaaggcaattatggacaagtgtggtgattctgatgattgcatctccacttctgaggctgctgaacgagcagctaaactttctgaggacaagattaagaatctgcccgtgcctggcgaagtagaattcataaatggtggccctccgtgtcagggtttttctgggatgaacagattcaatcaaagtccctggagcaaagtccagtgcgagatgatcttagcattcctgtcatttgcggagtatttccgtcctagattctttctcttagaaaatgttaggaactttgtctcgttcaacaaaggacagaccttcagattgacactggcatcactcctggagatgggataccaggtccgatttggaattttagaggcaggggcttatggtgttgcgcagtccaggaaaagggcattcatttgggccgctgcacctggagagactcttccattgcactggtctagaccc

SEQ ID NO: 107 - DNA, 662 bp, Oryza sativa
  misc_feature (1)..(662): C-terminal domain of OsMET1
ttgagctaggtggttcagacaaaccaaaggatgggcaatcagagaactgtcttgcaacacttgacatttttgctggttgtggaggtttatctgaaggattgcagcgatcaggattgtcacttactaaatgggctattgaatatgaagaacctgctggggatgcatttggtgaaaaccatccagaagctgcagtatttgtcgaaaactgcaatgtgattctgaaggcaattatggacaagtgtggtgattctgatgattgcatctccacttctgaggctgctgaacgagcagctaaactttctgaggacaagattaagaatctgcccgtgcctggcgaagtagaattcataaatggtggccctccgtgtcagggtttttctgggatgaacagattcaatcaaagtccctggagcaaagtccagtgcgagatgatcttagcattcctgtcatttgcggagtatttccgtcctagattctttctcttagaaaatgttaggaactttgtctcgttcaacaaaggacagaccttcagattgacactggcatcactcctggagatgggataccaggtccgatttggaattttagaggcaggggcttatggtgttgcgcagtccaggaaaagggcattcatttgggccgctgcacctggagagactctt

SEQ ID NO: 108 - DNA, 2596 bp, Arabidopsis thaliana
  misc_feature (1)..(2596): Ceres CDNA ID no. 23505366
ggttttgaatttgctctcaaaagcgacggagacgactgtttggctcggtgatttctcccgccgtttgggtttttcttaccggaatttccttctccttcgatggttagtctgcgctccagaaaagttattccggcttcggaaatggtcagcgacgggaaaacggagaaagatgcgtctggtgattcacccacttctgttctcaacgaagaggaaaactgtgaggagaaaagtgttactgttgtagaggaagagatacttctagccaaaaatggagattcttctcttatttctgaagccatggctcaggaggaagagcagctgctcaaacttcgggaagatgaagagaaagctaacaatgctggatctgctgttgctcctaatctgaatgaaactcagtttactaaacttgatgagctcttgacgcaaactcagctctactctgagtttctccttgagaaaatggaggatatcacaattaatgggatagaaagtgagagccaaaaagctgagcccgagaagactggtcgtggacgcaaaagaaaggctgcttctcagtacaacaatactaaggctaagagagcggttgctgctatgatttcaagatctaaagaagatggtgagaccatcaactcagatctgacagaggaagaaacagtcatcaaactgcagaatgaactttgtcctcttctcactggtggacagttaaagtcttatcagcttaaaggtgtcaaatggctaatatcattgtggcagaatggtttgaatggaatattagctgatcaaatgggacttggaaagacgattcaaacgatcggtttcttatcacatctgaaagggaatgggttggatggtccatatctagtcattgctccactgtctacactttcaaattggttcaatgagattgctaggttcacgccttccatcaatgcaatcatctaccatggggataaaaatcaaagggatgagctcaggaggaagcacatgcctaaaactgttggtcccaagttccctatagttattacttcttatgaggttgccatgaatgatgctaaaagaattctgcggcactatccatggaaatatgttgtgattgatgagggccacaggttgaaaaaccacaagtgtaaattgttgagggaactaaaacacttgaagatggataacaaacttctgctgacaggaacacctctgcaaaataatctttctgagctttggtctttgttaaattttattctgcctgacatctttacatcacatgatgaatttgaatcatggtttgatttttctgaaaagaacaaaaacgaagcaaccaaggaagaagaagagaaaagaagagctcaagttgtttccaaacttcatggtatactacgaccattcatccttcgaagaatgaaatgtgatgttgagctctcacttccacggaaaaaggagattataatgtatgctacaatgactgatcatcagaaaaagttccaggaacatctggtgaataacacgttggaagcacatcttggagagaatgccatccgaggtcaaggctggaagggaaagcttaacaacctggtcattcaacttcgaaagaactgcaaccatcctgaccttctccaggggcaaatagatggttcatatctctaccctcctgttgaagagattgttggacagtgtggtaaattccgcttattggagagattacttgttcggttatttgccaataatcacaaagtccttatcttctcccaatggacgaaacttttggacattatggattactacttcagtgagaaggggtttgaggtttgcagaatcgatggcagtgtgaagctggatgaaaggagaagacagattaaagatttcagtgatgagaagagcagctgtagtatatttctcctgagtaccagagctggaggactcggaatcaatcttactgctgctgatacatgcatcctctatgacagcgactggaaccctcaaatggacttgcaagccatggacagatgccacagaatcgggcagacgaaacctgttcatgtttataggctttccacggctcagtcgatagagacccgggttctgaaacgagcgtacagtaagctcaagctggaacatgtggttattggccaagggcagtttcatcaagaacgtgccaagtcttcaacacctttagaggaagaggacatactggcgttgcttaaggaagatgaaactgctgaagataagttgatacaaaccgatataagcgatgcggatcttgacaggttacttgaccggagtgacctgacaattactgcaccgggagagacacaagctgctgaagcttttccagtgaagggtccaggttgggaagtggtcctgcctagttcgggaggaatgctgtcttccctgaacagttaggacacattaataagccaggccttgaaaccacttctgtgttttttttttttttttccggaacatgatcggttacttttggctgggaggatttaattattagagggctcggaagtttttgtaagttaaagaactcacttaaaaccctgaaaacatgacagttaatggtgattagctctcaatgtgatgaaaacaattggccctc

SEQ ID NO: 109 - protein, 764 aa, Arabidopsis thaliana
  misc_feature (1)..(764): Ceres CDNA ID no. 23505366
MVSLRSRKVIPASEMVSDGKTEKDASGDSPTSVLNEEENCEEKSVTVVEEEILLAKNGDSSLISEAMAQEEEQLLKLREDEEKANNAGSAVAPNLNETQFTKLDELLTQTQLYSEFLLEKMEDITINGIESESQKAEPEKTGRGRKRKAASQYNNTKAKRAVAAMISRSKEDGETINSDLTEEETVIKLQNELCPLLTGGQLKSYQLKGVKWLISLWQNGLNGILADQMGLGKTIQTIGFLSHLKGNGLDGPYLVIAPLSTLSNWFNEIARFTPSINAIIYHGDKNQRDELRRKHMPKTVGPKFPIVITSYEVAMNDAKRILRHYPWKYVVIDEGHRLKNHKCKLLRELKHLKMDNKLLLTGTPLQNNLSELWSLLNFILPDIFTSHDEFESWFDFSEKNKNEATKEEEEKRRAQVVSKLHGILRPFILRRMKCDVELSLPRKKEIIMYATMTDHQKKFQEHLVNNTLEAHLGENAIRGQGWKGKLNNLVIQLRKNCNHPDLLQGQIDGSYLYPPVEEIVGQCGKFRLLERLLVRLFANNHKVLIFSQWTKLLDIMDYYFSEKGFEVCRIDGSVKLDERRRQIKDFSDEKSSCSIFLLSTRAGGLGINLTAADTCILYDSDWNPQMDLQAMDRCHRIGQTKPVHVYRLSTAQSIETRVLKRAYSKLKLEHVVIGQGQFHQERAKSSTPLEEEDILALLKEDETAEDKLIQTDISDADLDRLLDRSDLTITAPGETQAAEAFPVKGPGWEVVLPSSGGMLSSLNS

SEQ ID NO: 110 - DNA, 2256 bp, Populus balsamifera subsp. trichocarpa
  misc_feature (1)..(2256): Ceres GDNA ANNOT ID no. 1533431
atggggagcgaagtgaaaagtgaagcctctgctgattctcctacttccgttcttgaagacgaggagcaatgcaatttgaaagtagaagaagaggtgtttgtggaagcaaaaaatggggacgcgtctcttatatcaatatctatggcggaggaggaagagaagctgttgaaatccaggatgaaagaagaagagatagaaaaagcggcggcggaggaagctcaattggatgaaagccagtttaatagattggaccagcttttaactcaaactcagctctattcagagtttctgctggaaaagattgatcaaatcactgctaatggagcggagcaagagagtgaacctgtcgagcagaaaaagagaggtcgtggatcaaaaagaaaagccgctgcgcaatacaattctaggaaagccaagagagcagttacagcaatgcttacaagatcaaaagaagttgacaaggttgaggatgcaaatttgactgaggaagaaagagctgagaaagagcagagagaacttgtacctttgctaactggtgggaggttgaaatcttatcagattaaaggtgtgaaatggttgatctctctctggacaaatgggctcaatgggatccttgcagatcaaatggggctcggaaagactatccaaacaattggtttcctagcacatctgaaagggaatggtttaaatggaccctatatggttattgcccctctttccactctctcaaattgggtgaatgagatttcaaggtttgctccttctatggactcaattatctaccacggtagcaagaagcagagggatgagataaggaggaagcacatgcccagatcaattggctccaaattcccaattattgttacttcctatgagattgcattatcggatgcaaaaaagtatttgaggcattacccatggaagtatgttgtggttgatgaggggcaccggttgaaaaattcaaaatgcaaactgctgaaggagttgaaatacttgcacgtggacaataagctcttattaactgggacacctctgcaaaacaatttggctgagctttggtcactgttgaatttcattttgcctgatattttccaatcccatgaagaatttgagtcatggtttgatctgtcaggaaagtgcaataatgaaacaatgaaggaagaagtagaagaaaggcgaagggctcaggctgtaaccaaactccatgcaatattgcgtccctttctcctacggagaatgaaaactgatgtagagcagatgcttcctcggaagaaagagatcatcttatatgctaccctgactgagcatcagaagaaattccaggagcatttaatcaataagacactggaggactatttacgagagaagctggataccggacgtggcatgaaaggaaggcttaataatttgatgattcaacttcggaaaaattgctaccacccggatctcttagagtctgcctttgatggctcatatttctatccaccagttgaacagatagttgggaagtgtggcaaattccaattgctggatagattgctgaatcgtttgtttgcactccagcacaaagttctgattttctcccagtggactaaaattttggatataatggactattattttagtgagaaaggatttgaagtttgtagaattgatggctctgtgaagttagatgaaaggaagaaacagatccaggaattcaatgacgagaacagtcagtttaggatttttcttcttagcacaagagctggtggattgggtatcaacctcacttcagctgatacctgtatcctgtatgacagtgactggaaccctcaaatggatctgcaagccatggatagatgtcaccgaattgggcaaaccaagcctgttcatgtttacaggcttacaacagctcagtctgttgagggtcgtattttgaaaagagcttttagtaaattgaagcttgagcatgtggtcattggaaaaggacagtttcatcaagagcgaaccaagtctaccggcacagatctcatggaggaggagatgctggccctgcttcgggatgaagaaacagcagaagacaagttgatacagacagatatcagtgatgaagatttagagagggtgttggatcgcagtgatttggtcgtaggctcatcatcagatgacatagaaaacatggctgccgctgtttcaattcctctgaaagggcctggttgggaggtggttgtaccgactgccagtggaggcgtgctctctacccttaatagttag

SEQ ID NO: 111 - protein, 751 aa, Populus balsamifera subsp. trichocarpa
  misc_feature (1)..(751): Ceres GDNA ANNOT ID no. 1533431
MGSEVKSEASADSPTSVLEDEEQCNLKVEEEVFVEAKNGDASLISISMAEEEEKLLKSRMKEEEIEKAAAEEAQLDESQFNRLDQLLTQTQLYSEFLLEKIDQITANGAEQESEPVEQKKRGRGSKRKAAAQYNSRKAKRAVTAMLTRSKEVDKVEDANLTEEERAEKEQRELVPLLTGGRLKSYQIKGVKWLISLWTNGLNGILADQMGLGKTIQTIGFLAHLKGNGLNGPYMVIAPLSTLSNWVNEISRFAPSMDSIIYHGSKKQRDEIRRKHMPRSIGSKFPIIVTSYEIALSDAKKYLRHYPWKYVVVDEGHRLKNSKCKLLKELKYLHVDNKLLLTGTPLQNNLAELWSLLNFILPDIFQSHEEFESWFDLSGKCNNETMKEEVEERRRAQAVTKLHAILRPFLLRRMKTDVEQMLPRKKEIILYATLTEHQKKFQEHLINKTLEDYLREKLDTGRGMKGRLNNLMIQLRKNCYHPDLLESAFDGSYFYPPVEQIVGKCGKFQLLDRLLNRLFALQHKVLIFSQWTKILDIMDYYFSEKGFEVCRIDGSVKLDERKKQIQEFNDENSQFRIFLLSTRAGGLGINLTSADTCILYDSDWNPQMDLQAMDRCHRIGQTKPVHVYRLTTAQSVEGRILKRAFSKLKLEHVVIGKGQFHQERTKSTGTDLMEEEMLALLRDEETAEDKLIQTDISDEDLERVLDRSDLVVGSSSDDIENMAAAVSIPLKGPGWEVVVPTASGGVLSTLNS

SEQ ID NO: 112 - DNA, 2541 bp, Oryza sativa subsp. japonica
  misc_feature (1)..(2541): Public GI no. 51536001
atgtcggaagttagatccgtccctgggatgaaggtggaagcatccccggccgcggccgccgctgccaccgtggattccccgacatccgttctggaggatgatgagaagatttctgaatgtaagaatggggatgtcttggataccaccgaggcgattaagcaagaggaggatcatctcgatgtgcttattgaggagaaagttgatggttttgtggatgcaagttcttccctgaatgtggaacctgcggctaacaacagtgatttgtcacctctcactgtgccggtgaaggaagaaggtcagttattggagcctgtcaaggaggaaaaagctgatgattttgtggatgcagttccatctctaccgattgaccttgaggccaagaatggtgatgcatccctcatcactgatgcgatgaaggaggaggaagagaagttgcacgacgcccgggttaaggctgaagaagaagaggtagcacggaagagggaagaggctgcaagacttgcttttgatccaaatgcacgttttaacaagttggacgagctactatcacagacacaactctattcagaatttctacttgagaagatggaaacgatcgctgatgtggagggcgtccaaactcatgctgaagaggagccagtagaagagaagaagaatgggcgtggcagaaagaggaaagctacttctgcaccaaagtacaatgataaaaaagccaagaaagcagtggcagtcatgcttacaagatctcatgaagattgctcacctgaggattgtactctcacagaagaagaaaggtgggaaaaagagcaagccagacttgttcctctaatgactggcgggaagttgaagtcctaccagataaagggtgttaagtggttaatatcactttggcagaatgggcttaatgggatattggctgatcaaatgggccttgggaaaacaatacagactatcggttttcttgcccatctcaaggggaagggtcttgatggtccatacctgataattgctcccctatccactctctcaaactgggtgaacgagatctcaaggtttgttccatctatgactggtctgatttatcatggggacaaagcagcccgggcagagataaggagaaaattcatgcccaaaaccactggcccagattttccattaatcgtaacttcctatgagatggctatgtcagatgcaaagcatcttgctcactataagtggaagtatgttattgtggatgaggggcatcggctaaagaattccaagtgtttattattaagggagctaaagcgcctgccaatggataataagctccttttgactggaactcctcttcagaataatctagcagaactgtggtccctgctgaacttcattttgcctgatatattctcttcacaccaagaatttgagtcatggtttgacttttctgcgaaaggaggtgaagaagaacaagaggatagtgaagagaagagaaaagtcgatgttgtttcaaagcttcatgccatattgcgcccgttccttctaaggcggatgaaggaggatgtagagcatatgcttccacgaaagaaagagataatcatttacgctaacatgactgaccatcagaaacaaatccagaatcacttggttgaacaaacatttgatcaatacctgcatgaaaaatcagaaattgttctgcggaaacctggcattaaggcaaagctaaataatctgttaattcagttgaggaaaaattgcaaccatcctgatcttctggagtctgcgtatgactcatcaggtctgtatccacctgttgagaagctcttggaacagtgcggcaaatttcagctcttgaacagattgctaagtttgctgcttgcacggaagcacaaggttctaatattttcacagtggacaaaagttttggacattatcgagtactacttggaaacaaaaggccttcaggtttgccgaattgatgggagtgttaagttggaagagaggaggaggcagatagcagaatttaatgacttgaacagcagcatgaatatctttattctaagcacacgggctggcgggcttggtatcaatcttacttcagcggatacctgtatcctatatgatagtgactggaatcctcagatggatttgcaggccatggatcgatgtcaccggattggtcaaacacgcccagttcacgtctatcggttagcaacatcacattctgttgagggccggatcatcaagaaagcatttggcaagttgaggttggagcatgtggtgatcgggaagggtcagtttgaacaagacagagccaagcctaatgccctagatgaagcggagttgctggcgctgctcagggacgagcagggcgacgaggaccggatgatccagacggacatcagcgatgaggacctcctgaaggtgatggaccggagcgaccttacaggccctcctgccaatgccgacgccgcccctctcgtccccctgaagggacccggctgggaggtggtggtgccgacgaagagcggcggcggaatgctgacgtcgctcaccagttag

SEQ ID NO: 113 - protein, 846 aa, Oryza sativa subsp. japonica
  misc_feature (1)..(846): Public GI no. 51536001
MSEVRSVPGMKVEASPAAAAAATVDSPTSVLEDDEKISECKNGDVLDTTEAIKQEEDHLDVLIEEKVDGFVDASSSLNVEPAANNSDLSPLTVPVKEEGQLLEPVKEEKADDFVDAVPSLPIDLEAKNGDASLITDAMKEEEEKLHDARVKAEEEEVARKREEAARLAFDPNARFNKLDELLSQTQLYSEFLLEKMETIADVEGVQTHAEEEPVEEKKNGRGRKRKATSAPKYNDKKAKKAVAVMLTRSHEDCSPEDCTLTEEERWEKEQARLVPLMTGGKLKSYQIKGVKWLISLWQNGLNGILADQMGLGKTIQTIGFLAHLKGKGLDGPYLIIAPLSTLSNWVNEISRFVPSMTGLIYHGDKAARAEIRRKFMPKTTGPDFPLIVTSYEMAMSDAKHLAHYKWKYVIVDEGHRLKNSKCLLLRELKRLPMDNKLLLTGTPLQNNLAELWSLLNFILPDIFSSHQEFESWFDFSAKGGEEEQEDSEEKRKVDVVSKLHAILRPFLLRRMKEDVEHMLPRKKEIIIYANMTDHQKQIQNHLVEQTFDQYLHEKSEIVLRKPGIKAKLNNLLIQLRKNCNHPDLLESAYDSSGLYPPVEKLLEQCGKFQLLNRLLSLLLARKHKVLIFSQWTKVLDIIEYYLETKGLQVCRIDGSVKLEERRRQIAEFNDLNSSMNIFILSTRAGGLGINLTSADTCILYDSDWNPQMDLQAMDRCHRIGQTRPVHVYRLATSHSVEGRIIKKAFGKLRLEHVVIGKGQFEQDRAKPNALDEAELLALLRDEQGDEDRMIQTDISDEDLLKVMDRSDLTGPPANADAAPLVPLKGPGWEVVVPTKSGGGMLTSLTS

SEQ ID NO: 114 - DNA, 2649 bp, Triticum monococcum
  misc_feature (1)..(2649): Public GI no. 45357056
atggcggcgaccgcctacgagcgccgcctgctcgccgccgccgacctcgtcctctccgccgacggcaagtcccagctcccccgcctctcctccgccgacctcggcgtcacggccgacctcaagccgcaccagctggacggcgtcgactggctcatccgccgctaccacctcggcgtcaacgtcctgctcggcgatgagatggggctggggaagaccctgcaggcgatttctctgctgagctacctgaagatcaaatccatcgcccccggaccgttcctggtgttatgccctctaagtgtgacggatggctggttgtcggagttcggtaaattctgccccaccttgaaggtcatccagtatgttggcgataagccgcatcgtcgtcagatacggagaacgatccatgaagatgtccaaaaatcttcacattcagatgaattaccatttgatgtgatgctgacgagctatgacatagccttaatggatcaagattttctttcccaagttccttggctctatgtggtgattgatgaggcccagcgtcttaaaaatccatcaagtgtactgtataatgttcttgaagaacgctttatgatgccaaggcgtctactactaacaggcactcctgtccagaataacctttctgaactatgggcgttgatgcacttctgcatgccttcagtttttgggcccctagatgaatttctttctactttcaaggaagcagggaatttgttatcaggtagtgaagctaataaagcaaacagacaattcaagatccttaaacatatactcagagcatttatgctccggcgaacaaaggctttactaattgagagtggaattctggaattgcctccattaactgagctgacagtgatggtacctctggcacccttacagaagaagatatacttatcagtgttgagaaaagagctgcaaacacttctttcatttactggtggatcttctcgtcatcaatctctgcagaacattgtagtacaactgcggaaagcttgcagtcatccatatctattcagtggtattgagcctgagccttatgaggaaggggagcatttggttcaggctagtggaaagctcattgtgctagaccttgttcttgagaagctccacaggttaggtcatcgtgttgtgctatttgctcagatgactcagacgctggacattctgcaggatttcttagaactgcgtaattacacgtatgagcgcctggatggttcagtacgtgccgaggaacggtttgcagcaatcagaaatttcagctctcaacctaccaaaggtgttgtaagagatgacagtaatccaagtggagcttttgttttcatgatatcaactagagcagggggtgttgggcttaatctcattggtgctgatactgttattttttatgagcaagactggaatcctcaggctgacaaacaggctttgcagcgtactcaccgcattggacagttgaatcatgtattatcaataaatttggtatcgcaacgcacgattgaagaggtcatcatgcgaagagcagagagaaaacttaagcttagccacaatatttttggagataaggatgcaactgatgggaaaggaaatgacttgggaaatgaagctaatgacatgtgttcgattatatttggcttacaccagtttgaccccgcagatacagctgcggaaacaatcaatgaggagacactggaaaaattgaaatcaatgtcagaaaatgttatcaaaatgcgtactcatgaaccttcagaaaaggatgaccgagcatttgaaatcaatccagatttgactgagggcagcgggcttgtaattacaagagcttgtgattctattagcattgatccaggggttgacgaagctgcatacctatcctgggtagagaagtttaaggaggcttcacattctattgaagatgtcccagttgaattagaaaggcaaagacctgcacctgaagataagctcttgaaacgtgaagctaataagaaaaaggctgaagaaaagagattggccaagtggaaagatttggggtaccagacattaggggtcaaagttcctgataacataccaaatcagaatatttctgactcaggatccgtccaacttgtatatggagattgcactgatccatcaaaagtttgcgctgcaaagcctgccattatattcagttgtgtagataattctggaacatggggtcatggaggaatgtttgatgctttgactagtctctcaacgtacattccagatgcttatcatcgtgcttctgaagtcgacgatcttcacatgggggatcttcacttgattcagctagatgaagccaactgtagccggaatttggatgcacctctatgggtggcactagctgttgtgcagtcttataatccgaaacgtaagattccaagaagtgaaatctcaatgtctgacttggagctgtgtttgtcaaaggcggccttctcagctgctcagcgttctgcgagtatccacatgcctcggattggtcagcgaagtggctcccagcgatcagagtggtataccatagaacgcctgctcaggaaatacgcatcgcttcatggcatcgacattttcgtgtactacttccggcgaccaaccaggcaacagccagactcagattaa

SEQ ID NO: 115 - protein, 882 aa, Triticum monococcum
  misc_feature (1)..(882): Public GI no. 45357056
MAATAYERRLLAAADLVLSADGKSQLPRLSSADLGVTADLKPHQLDGVDWLIRRYHLGVNVLLGDEMGLGKTLQAISLLSYLKIKSIAPGPFLVLCPLSVTDGWLSEFGKFCPTLKVIQYVGDKPHRRQIRRTIHEDVQKSSHSDELPFDVMLTSYDIALMDQDFLSQVPWLYVVIDEAQRLKNPSSVLYNVLEERFMMPRRLLLTGTPVQNNLSELWALMHFCMPSVFGPLDEFLSTFKEAGNLLSGSEANKANRQFKILKHILRAFMLRRTKALLIESGILELPPLTELTVMVPLAPLQKKIYLSVLRKELQTLLSFTGGSSRHQSLQNIVVQLRKACSHPYLFSGIEPEPYEEGEHLVQASGKLIVLDLVLEKLHRLGHRVVLFAQMTQTLDILQDFLELRNYTYERLDGSVRAEERFAAIRNFSSQPTKGVVRDDSNPSGAFVFMISTRAGGVGLNLIGADTVIFYEQDWNPQADKQALQRTHRIGQLNHVLSINLVSQRTIEEVIMRRAERKLKLSHNIFGDKDATDGKGNDLGNEANDMCSIIFGLHQFDPADTAAETINEETLEKLKSMSENVIKMRTHEPSEKDDRAFEINPDLTEGSGLVITRACDSISIDPGVDEAAYLSWVEKFKEASHSIEDVPVELERQRPAPEDKLLKREANKKKAEEKRLAKWKDLGYQTLGVKVPDNIPNQNISDSGSVQLVYGDCTDPSKVCAAKPAIIFSCVDNSGTWGHGGMFDALTSLSTYIPDAYHRASEVDDLHMGDLHLIQLDEANCSRNLDAPLWVALAVVQSYNPKRKIPRSEISMSDLELCLSKAAFSAAQRSASIHMPRIGQRSGSQRSEWYTIERLLRKYASLHGIDIFVYYFRRPTRQQPDSD

SEQ ID NO: 116 - DNA, 3261 bp, Chlamydomonas reinhardtii
  misc_feature (1)..(3261): Public GI no. 68144413
atgtcgctgctggacgatcctaactttgtagggcaagacgaggatgatgacgtggcgaaccagctggccgaggaggacactcaggaccaggcggaggacgacgacgacgacgagagcgtaaagacggaggatgtggactatgagccggagctcggggatgacgcggtggtctcccctgcaaagctggcagttgcgaaggcggaaacggaacgccttaagcatcaggcaaagcagaagaaggagatgctggagcgcatgcgcgagcagcagaaccagctggccacgatgggcgacgcggagcgggcgcggcaccgcatcaacttcctgttgaagcaggcggagattttccagcacttcgcgtcggactcggccgtgaaggaggccaagaaggccaagaccaagggccgcggccagcgcaaggaggaggacgaggacgctgagctgctgcaggacgaggacgacggcggcacgcacgcgggccaccgcctgcaggtgcagccgtccatcatcacgggcggcacgctgcgcgagtaccagatgcagggcctcaactggatgatccacctgtacgacaacggtatcaacggcatcctggccgatgagatgggtctgggcaagacgctccagaccatctcgctggtggcctacctgtacgagtaccgcggcatcactgggccgcacatcgtcatcacgcccaagtccacgctgggcaactgggtgaacgagttcaagcgcttcgcgcccatcatccgcgtcaccaagttccacggcaacgcggacgagcgcatgatccagaaggagaccacctgcgcgcccggccgcttcgacgtggttgtgacgtcatacgagatggtgatcaaggagaagaaccactttaagcgcttccactggcgctacatcatcatcgacgaggcgcaccgcatcaagaacgagaacagccgcctgtcgctggtggtccgccagctcaagaccaactaccggctgctgatcacgggcacgccgctacagaacaacctgcacgagctgtgggcgctgctcaacttcctgctgcccgagatcttcagcagcgcggagaagttcgaggagtggttcagcctgggcgacggaagcaaggagaaggaggcggaggtggtgcagcagctgcacaaggtgctgcgccccttcctgctgcgccgtgtcaagtcagacgtggagcgcggcctgccgcccaagaaggagaccatcctcaagatcggcatgagcgagatgcagaagaagtggtacgcggctctgctgcagaaggacgtggacgcgctcaacggcggcgccgaccgcgccaagctgctcaacgtggtcatgcagctgcgcaagtgctgcaaccacccctacctgttccagggcgcggagcccggcccgcccttcattaccggcgagcaccttgttgagaacagcggcaagctggtgctgctggacaagctgctgccgcgcctcaaggagcgcgagagccgcgtgctcatcttcagccagatgacgcgcatgatcgacatcctggaggactactgcctgtacaggggctacggctactgccgcattgacggcaacacggacggcgaggcgcgcgacaacatgattgacgagttcaaccgccccaactcctccaagttcatcttcctgctgtccacccgcgccggcggcctgggcatcaacctggccaccgccgacatcgtggtgctgtacgacagcgactggaacccgcagatggacctgcaggccatggaccgcgcgcaccgcatcggccagaagaaggaggtgcaggtgttccgcttctgcatcgagaactccatcgaggagaaggtgatcgagaaggcctacaagaagctgcgcctggacgcgctggtcatccagcagggccgcctcacggagaactcggccaccaaggtgaacaaggacgacctgattaacatggtgcgctacggcgccgagctggtgttcagctccgactcatccaacatcacggacgctgacattgacgccatcatcaagaagggcgagcgcgacacagccgacctgaaccagaagatgcagcagttcacggacaacgccatgaagttcaccatggacggcggcatggtgtacgacttcaaggacgccgacgacgagcgggccgacattggcgacctcaaggccatcatgggctccaactggattgacccgcccaagcgcgagcgcaagcggcaccagctcaactacaacgacgccgactactacaagaacgcgctcaagaccggcgccaaggacccgcgcgcgcagcggctgcccaagatgccgcagctgcaggacttccagttcttcaacgtgccgcgcatccaggagctgtacgagaaggagcacaaccacgaggagcacaagcgtgcgctgaccaaccgggaggcggcgctgcgcaaccagggcgccagcgacgaggccgtggcggaggcgctggcggacaaggaggacgacccgcagccgctcacggaggaggagctggaggagcgcgagaagctgctggaggagggcttcaaggagtggaccaagcgcgacttcaacgcgttcgtgcgcgcctgcgagaagtacgggcgtgagaacatcccccagatcgctgcggaggtggatggcaagacggaggacgaagtgcgcgagtacgccaaggtgttctggcagcgctaccgcgagctggcggactacgagaaggtgatcaagaacattgagcggggcgagcagaagatccagcgccagcaggacatcatgaacgccatcgccgccaagctggagcggtacaagaacccctggcaggagctcaagatccagtacggcgccaacaagggcaaggcctacacggaggaggaggaccgcttcatcctgtgcatggtgcacaagctgggctacggcaactgggacgacctcaaggccgagattcgcaagagctggcgcttccgcttcgactggttcttcaagagccgcacgccgcaggagctgggccgccgctgcgagacgctgatccgcctgatcgagaaggagaacgaggacaatgccgacgcgccgccgcgcaccggcggtggccgcggaggcggtcgcggagggcggggcggcggccgtggaggcggcgacggccctgggcgcaagcgcaagggccaggtggcgacgccagcggccggcacgccagcggtgggcggcacgccgcagcccggcagcgaggaggggtcaccgcagccgccggaccgcaagaagggccgcatggcgtaa

SEQ ID NO: 117 - protein, 1086 aa, Chlamydomonas reinhardtii
  misc_feature (1)..(1086): Public GI no. 68144413
MSLLDDPNFVGQDEDDDVANQLAEEDTQDQAEDDDDDESVKTEDVDYEPELGDDAVVSPAKLAVAKAETERLKHQAKQKKEMLERMREQQNQLATMGDAERARHRINFLLKQAEIFQHFASDSAVKEAKKAKTKGRGQRKEEDEDAELLQDEDDGGTHAGHRLQVQPSIITGGTLREYQMQGLNWMIHLYDNGINGILADEMGLGKTLQTISLVAYLYEYRGITGPHIVITPKSTLGNWVNEFKRFAPIIRVTKFHGNADERMIQKETTCAPGRFDVVVTSYEMVIKEKNHFKRFHWRYIIIDEAHRIKNENSRLSLVVRQLKTNYRLLITGTPLQNNLHELWALLNFLLPEIFSSAEKFEEWFSLGDGSKEKEAEVVQQLHKVLRPFLLRRVKSDVERGLPPKKETILKIGMSEMQKKWYAALLQKDVDALNGGADRAKLLNVVMQLRKCCNHPYLFQGAEPGPPFITGEHLVENSGKLVLLDKLLPRLKERESRVLIFSQMTRMIDILEDYCLYRGYGYCRIDGNTDGEARDNMIDEFNRPNSSKFIFLLSTRAGGLGINLATADIVVLYDSDWNPQMDLQAMDRAHRIGQKKEVQVFRFCIENSIEEKVIEKAYKKLRLDALVIQQGRLTENSATKVNKDDLINMVRYGAELVFSSDSSNITDADIDAIIKKGERDTADLNQKMQQFTDNAMKFTMDGGMVYDFKDADDERADIGDLKAIMGSNWIDPPKRERKRHQLNYNDADYYKNALKTGAKDPRAQRLPKMPQLQDFQFFNVPRIQELYEKEHNHEEHKRALTNREAALRNQGASDEAVAEALADKEDDPQPLTEEELEEREKLLEEGFKEWTKRDFNAFVRACEKYGRENIPQIAAEVDGKTEDEVREYAKVFWQRYRELADYEKVIKNIERGEQKIQRQQDIMNAIAAKLERYKNPWQELKIQYGANKGKAYTEEEDRFILCMVHKLGYGNWDDLKAEIRKSWRFRFDWFFKSRTPQELGRRCETLIRLIEKENEDNADAPPRTGGGRGGGRGGRGGGRGGGDGPGRKRKGQVATPAAGTPAVGGTPQPGSEEGSPQPPDRKKGRMA

SEQ ID NO: 118 - DNA, 3336 bp, Oryza sativa subsp. indica
  misc_feature (1)..(3336): Public GI no. 37542688
atgagcagcctggtggagcggctgcgggtgcggtcggagaagcggccgctgtacacgctcgatgagtccgacgacgacctccgcgcgcggcgggggcgggaaggggagggatcggcacagcgacggccccaccgagcggatcgagcgggaggacgcgttattcacctgtgtgtgcagaaagaagatgcttgccagaaatgtggagaaaatgacaatctagtaccatgttcaacttgtacttatgcatttcacaggaaatgtttggttcctcgcttaaatatcacatctgataaatggagctgccctgaatgtgttagccctcttacggaaatggaaaagatattagactgtgaagaaacaaaacctgatgcttctgaagagactagttcttctgagtccggatcaaagaagaaacctgtcaaacgatatcttataaaatggaaaggaatatcacaccttcactgcacttgggtttcagaaagtgaatatttggaaactgccaagatatacccccggctgaaaactagactcaataacttccataagcaaatggattcaacggataagtctgatgatgactattctgcaattagacctgagtggactactgttgacaggatccttgctaccagaaaaagctctactggtgaaagggagtactatgtgaaatggaaggaactaacatacgacgaatgtacttgggaaaatgactccgacattgcagtctttcagcctcagattgaacgcttcaatgagattcagtccaggaggaagaagtctactgacaagtgcaagagtgtcactcgtgagatacgtcaatataaggagagcccgaagtttctctctggtgggacactacatccctaccagcttgaagggttaaacttcttacgctattcttggtaccataataaacgtgtaatacttggggacgagatgggtcttggtaaaacgatacagagtattgcctttttgggctctctatttgtggacaagcttggtcctcatctggtcgttgcacccctttcaaccttgcgaaattgggagcgtgaatttgcaacttgggcacctcaaatgaatgttgtaatgtattttggatccgcagcttctcgtgaaattattaggaagtatgaattttactaccccaaagagaaacctaagaagcttaagaaaaagaaatcatctccatctaatgaagacaagaaacaatcaagaataaaatttgatgtccttttgacatcttatgagatgatcaatatggattctactgttctaaaaaccatagaatgggaatgcatgattgtggatgaggggcatcgtttgaagaacaaagattccaagttgttcggtcaacttaaagagtatcatactaagcatcgtgttctcttaacgggaaccccagttcagaacaacctggatgaacttttcatgcttatgcacttccttgagggtgacagttttggtagtatagctgatctccaagaggaattcaaggacataaaccaggacaaacaagttgagaagctccatggaatgctgaagccgcatcttcttcggcgattcaagaaagatgttatgaaagaacttcctccgaaaaaggagctgattttacgtgttgagcttacaagcaaacaaaaagagtactataaagcaattctcaccaagaattatgaagtgttaactcgtcgtagtggtggacatgtttcacttattaatgttgtaatggaactacgcaaactttgttgccatgcattcatgacagatgaacccgaagagcctgccaattcagaagaagctttaaggaggcttttagaatcttctggaaaaatggagctgcttgacaagatgatggtgaaactgaaagagcagggtcacagggttcttatttattcacagttccagcacatgttggacttacttgaggattatttaagctaccggaaatggagttatgagcgtattgatggaaaaataggtggtgctgagaggcagatacgaattgatcgcttcaatgctaaaaattctactaggttttgctttcttctttcaaccagagctggtggtcttggtataaatctggcaactgccgatactgtaattatttatgacagtgattggaatcctcatgctgatttacaagctatggcgagagcccatcgcttagggcagacaagcaaggttatgatatataggcttgttagtcgtggtacaattgaggagcggatgatgcaacttacaaagaaaaaaatggtattggagcacttagttgttgggcgtctcacgaaaggcactaatattgtccaggaggagttggatgatattattcggcatggctcaaaggaactttttgacgatgaaaatgatgaagccggaaaatcttgccaaatccattatgatgatgctgcgattgatagattattggaccgtgaccaagctgacggggaagagcctgtggaagatgaagaagaagacgaatttctaaaaggattcaaggttgctaactttgagtacatagatgaagctaaggctttagctgcaaaagaggaggaggcacgcaaaaaggccgaagctgaagctgcaaactctgacagagcaaatttttgggataaattattgaaggacagatacgatgtacaaaaagttgaagagcatactactatgggaaaaggaaaaaggagccgcaaacagatggctgctgctgatgaggacgacattaccggcctgcatgacatgagttctgaagatgatgattactcttatgatgatgatgtgtcagataacgacacaagtttacaatcaggccttgctgggaggaggggcccatattccaagaaaaaacaacgtaatgttgattcacttccattcatggagggcgaaggacgggctttgagagtttatgggttcaaccaaattcagcgaacacaattccttcaaacgcttatgagatatggttttcagaactacgattggaaggaatttactcctcgattgaaggggaaaagtgttgaggaaatccagagatatgctgaacttgtcatgatccatcttcttgaggacatcaatgactcaggatattatgcagatggcgtgccaaaggaaatgcgcacagatgagacattggtcaggctagccaacatatcacttgtggaagagaaggtggctgccatggaacaaggaaaaataacaaaactctttcccagctatttgttatatgaatttcctagcttagttggtggaagagtatggaaagcggaacaggatctactgttgctgaaagcattaataaaggagccacccactgttaaggcggaggacaaaatggaaatcgatagttaa

SEQ ID NO: 119 - protein, 1111 aa, Oryza sativa subsp. indica
  misc_feature (1)..(1111): Public GI no. 37542688
MSSLVERLRVRSEKRPLYTLDESDDDLRARRGREGEGSAQRRPHRADRAGGRVIHLCVQKEDACQKCGENDNLVPCSTCTYAFHRKCLVPRLNITSDKWSCPECVSPLTEMEKILDCEETKPDASEETSSSESGSKKKPVKRYLIKWKGISHLHCTWVSESEYLETAKIYPRLKTRLNNFHKQMDSTDKSDDDYSAIRPEWTTVDRILATRKSSTGEREYYVKWKELTYDECTWENDSDIAVFQPQIERFNEIQSRRKKSTDKCKSVTREIRQYKESPKFLSGGTLHPYQLEGLNFLRYSWYHNKRVILGDEMGLGKTIQSIAFLGSLFVDKLGPHLVVAPLSTLRNWEREFATWAPQMNVVMYFGSAASREIIRKYEFYYPKEKPKKLKKKKSSPSNEDKKQSRIKFDVLLTSYEMINMDSTVLKTIEWECMIVDEGHRLKNKDSKLFGQLKEYHTKHRVLLTGTPVQNNLDELFMLMHFLEGDSFGSIADLQEEFKDINQDKQVEKLHGMLKPHLLRRFKKDVMKELPPKKELILRVELTSKQKEYYKAILTKNYEVLTRRSGGHVSLINVVMELRKLCCHAFMTDEPEEPANSEEALRRLLESSGKMELLDKMMVKLKEQGHRVLIYSQFQHMLDLLEDYLSYRKWSYERIDGKIGGAERQIRIDRFNAKNSTRFCFLLSTRAGGLGINLATADTVIIYDSDWNPHADLQAMARAHRLGQTSKVMIYRLVSRGTIEERMMQLTKKKMVLEHLVVGRLTKGTNIVQEELDDIIRHGSKELFDDENDEAGKSCQIHYDDAAIDRLLDRDQADGEEPVEDEEEDEFLKGFKVANFEYIDEAKALAAKEEEARKKAEAEAANSDRANFWDKLLKDRYDVQKVEEHTTMGKGKRSRKQMAAADEDDITGLHDMSSEDDDYSYDDDVSDNDTSLQSGLAGRRGPYSKKKQRNVDSLPFMEGEGRALRVYGFNQIQRTQFLQTLMRYGFQNYDWKEFTPRLKGKSVEEIQRYAELVMIHLLEDINDSGYYADGVPKEMRTDETLVRLANISLVEEKVAAMEQGKITKLFPSYLLYEFPSLVGGRVWKAEQDLLLLKALIKEPPTVKAEDKMEIDS

SEQ ID NO: 120 - DNA, 4720 bp, Arabidopsis thaliana
  misc_feature (1)..(4720): Ceres CDNA ID no. 23631543
tgacgtagcgaccaattagggtttcgcaatcttccagtagatttcgcttcgcaacggattttgaaaatggtggaaaatggggctaaagctgcgaagcgaaagaagagaccacttccagagattcaagaggtagaagatgtacctaggacgaggagaccaaggcgtgctgcagcgtgtaccagtttcaaggagaaatctattcgagtctgtgagaaatctgctactattgaagtaaagaaacagcagattgtggaggaagagtttctcgcgttacggttaacggctctggaaactgatgttgaagatcgtccaaccaggagactgaatgattttgttttgtttgattcagatggagttccacaacctctggagatgttggagattcatgacatattcgtttcaggtgctatcttaccttcagatgtgtgtactgataaggagaaagagaagggtgtgaggtgtacatcgtttggacgggttgagcattggagtatctctggttatgaagatggttcccctgttatttggatctcaacggaattggcggattatgattgtcgtaaacctgctgctagctacaggaaggtttatgattacttctatgagaaagctcgtgcttcagtggctgtgtataagaaattgtccaagtcatctggtggggatcctgatataggtcttgaggagttacttgcggcggttgtcagatcaatgagcagtggaagcaagtacttttctagtggtgcggcaatcatcgattttgttatatcccagggagattttatatataaccaactcgctggtttggatgagacagccaagaaacatgaatcaagctatgttgagattcctgttcttgtagctctcagagagaagagtagtaagattgacaagcctctgcagagggaaagaaacccatctaatggtgtgaggattaaagaagtttctcaagttgcggagagcgaggccttgacatctgatcaactggttgatggtactgatgatgacagaagatatgctatactcttacaagacgaagagaataggaaatctatgcaacagcccagaaaaaacagcagctcaggttctgcttcaaatatgttctacattaagataaatgaagatgagattgccaatgattatcctctcccatcgtactataagacctccgaagaagaaacagatgaacttatactttatgatgcttcctatgaggttcaatctgaacacctgcctcacaggatgcttcacaactgggctctttataactctgatttacgattcatatcactggaacttctaccgatgaaacaatgtgatgatattgatgtcaacatttttgggtcaggtgtggtgactgatgataatggaagttggatttctttaaacgatcctgacagcggttctcagtcacacgatcctgatgggatgtgcatattcctcagtcaaattaaagaatggatgattgagtttgggagcgatgatattatctccatttctatacgaacagatgtggcctggtaccgtcttgggaaaccatcaaaactttatgccccttggtggaaacctgttctgaaaacagcaagggttgggataagcattcttacttttcttagggtggaaagtagggttgctaggctttcatttgcagatgtcacaaaaagactgtctgggttacaggcgaatgataaagcttacatttcttctgaccccttggctgttgagagatatttggtcgtccatgggcaaattattttacagctttttgcagtttatccggacgacaatgtcaaaaggtgtccatttgttgttggtcttgcaagcaaattggaggataggcaccacacaaaatggatcatcaagaagaagaaaatttcgctgaaggaactgaatctgaatccaagggcaggcatggcaccagtagcatcgaagaggaaagctatgcaagcaacaacaactcgcctggtcaacagaatttggggagagttttactccaattactctccagaggatccattgcaggcgactgctgcagaaaatggggaggatgaggtggaagaggaaggcggaaatggggaggaagaggttgaagaggaaggtgaaaatggtctcacagaggacactgtaccagaacctgttgaggttcagaagcctcatactcctaagaaaatccgaggcagttctggaaaaagggaaataaaatgggatggtgagagtctaggaaaaacttctgctggcgagcctctctatcaacaagcccttgttggaggggaaatggtggctgtaggtggcgctgtcaccttggaagttgatgatccagatgaaatgccggccatctattttgtggagtacatgttcgaaagtacagatcactgcaaaatgttacatggtagattcttacaaagaggatctatgactgttctggggaatgctgctaacgagagggaactattcctgactaatgaatgcatgactacacagctcaaggacattaaaggagtagccagttttgagattcgatcaaggccatgggggcatcagtataggaaaaagaacatcactgcggataagcttgactgggctagagcattagaaagaaaagtaaaagatttgccaacagagtattactgcaaaagcttgtactcacctgagagagggggattctttagtcttccactaagtgatattggtcgcagttctgggttctgcacttcatgtaagataagggaggatgaagagaagaggtctacaattaaactaaatgtttcaaagacaggctttttcatcaatgggattgagtattctgttgaggattttgtctatgtcaaccctgactctattggtgggttgaaggagggtagtaaaacttcttttaagtctgggcgaaacattgggttaagagcgtatgttgtttgccaattgctggaaattgttccaaaggaatctagaaaggctgatttgggttcctttgatgttaaagtgagaaggttttataggcctgaggatgtttctgcagagaaggcctatgcttcagacatccaagaattgtatttcagccaggacacagttgttctccctccaggtgctctagagggaaaatgtgaagtaagaaagaaaagtgatatgcccttatcccgtgaatatccaatatcagaccatattttcttctgtgatcttttctttgacacctccaaaggttctctcaagcagctgcccgccaatatgaagccaaagttctctactattaaggacgacacacttttaagaaagaaaaagggaaagggagtagagagtgaaattgagtctgagattgtcaagcctgttgagccacctaaagagattcgtctggctactctagatatttttgctggttgtggtggcctgtctcatggactgaaaaaggcgggtgtatctgatgcaaagtgggcgattgagtatgaagagccagctgggcaggcttttaaacaaaaccatcctgagtcaacagtttttgtcgacaactgcaatgtgattcttagggctataatggagaaaggtggagatcaagatgattgtgtctctactacagaggcaaatgaattagcagctaaactaactgaggagcagaagagtactctgccactgcctggtcaagtggacttcatcaatggtggacctccatgtcagggattttctggtatgaacaggttcaaccaaagctcttggagtaaagttcagtgtgaaatgatattagcattcttgtcctttgctgactatttccggccaaggtattttcttctggagaacgtgaggacctttgtgtcattcaataaagggcagacatttcagcttactttggcttcccttctcgaaatgggttaccaggtgagatttggaatcctggaggccggtgcatatggagtatcccaatctcgtaaacgagctttcatttgggctgctgcaccagaagaagttctccctgaatggcctgagccgatgcatgtctttggtgttccaaagttgaaaatctcactatctcaaggtttacattatgctgctgttcgtagtactgcacttggtgcccctttccgtccaatcaccgtgagagacacaattggtgatcttccatcagtagaaaacggagactctaggacaaacaaagagtataaagaggttgcagtctcgtggttccaaaaggagataagaggaaacacgattgctctcactgatcatatctgcaaggctatgaatgagcttaacctcattcgatgcaaattaatcccaactaggcctggggctgattggcatgacttgccaaagagaaaggttacgttatctgatgggcgcgtagaagaaatgattcctttttgtctcccaaacacagctgagcgccacaacggttggaagggactatatgggagattagattggcaaggaaactttccgacttccgtcacggatcctcagcccatgggtaaggttggaatgtgctttcatcctgaacagcacagaatccttacagtccgtgaatgcgcccgatctcaggggtttccggatagctacgagtttgcagggaacataaatcacaagcacaggcagattgggaatgcagtccctccaccattggcatttgctctaggtcgtaagctcaaagaagccctacatctcaagaagtctcctcaacaccaaccctagataaccacccaaatttggcatttcctttttcaataatattagtcattat

SEQ ID NO: 121 - protein, 1534 aa, Arabidopsis thaliana
  misc_feature (1)..(1534): Ceres CDNA ID no. 23631543
MVENGAKAAKRKKRPLPEIQEVEDVPRTRRPRRAAACTSFKEKSIRVCEKSATIEVKKQQIVEEEFLALRLTALETDVEDRPTRRLNDFVLFDSDGVPQPLEMLEIHDIFVSGAILPSDVCTDKEKEKGVRCTSFGRVEHWSISGYEDGSPVIWISTELADYDCRKPAASYRKVYDYFYEKARASVAVYKKLSKSSGGDPDIGLEELLAAVVRSMSSGSKYFSSGAAIIDFVISQGDFIYNQLAGLDETAKKHESSYVEIPVLVALREKSSKIDKPLQRERNPSNGVRIKEVSQVAESEALTSDQLVDGTDDDRRYAILLQDEENRKSMQQPRKNSSSGSASNMFYIKINEDEIANDYPLPSYYKTSEEETDELILYDASYEVQSEHLPHRMLHNWALYNSDLRFISLELLPMKQCDDIDVNIFGSGVVTDDNGSWISLNDPDSGSQSHDPDGMCIFLSQIKEWMIEFGSDDIISISIRTDVAWYRLGKPSKLYAPWWKPVLKTARVGISILTFLRVESRVARLSFADVTKRLSGLQANDKAYISSDPLAVERYLVVHGQIILQLFAVYPDDNVKRCPFVVGLASKLEDRHHTKWIIKKKKISLKELNLNPRAGMAPVASKRKAMQATTTRLVNRIWGEFYSNYSPEDPLQATAAENGEDEVEEEGGNGEEEVEEEGENGLTEDTVPEPVEVQKPHTPKKIRGSSGKREIKWDGESLGKTSAGEPLYQQALVGGEMVAVGGAVTLEVDDPDEMPAIYFVEYMFESTDHCKMLHGRFLQRGSMTVLGNAANERELFLTNECMTTQLKDIKGVASFEIRSRPWGHQYRKKNITADKLDWARALERKVKDLPTEYYCKSLYSPERGGFFSLPLSDIGRSSGFCTSCKIREDEEKRSTIKLNVSKTGFFINGIEYSVEDFVYVNPDSIGGLKEGSKTSFKSGRNIGLRAYVVCQLLEIVPKESRKADLGSFDVKVRRFYRPEDVSAEKAYASDIQELYFSQDTVVLPPGALEGKCEVRKKSDMPLSREYPISDHIFFCDLFFDTSKGSLKQLPANMKPKFSTIKDDTLLRKKKGKGVESEIESEIVKPVEPPKEIRLATLDIFAGCGGLSHGLKKAGVSDAKWAIEYEEPAGQAFKQNHPESTVFVDNCNVILRAIMEKGGDQDDCVSTTEANELAAKLTEEQKSTLPLPGQVDFINGGPPCQGFSGMNRFNQSSWSKVQCEMILAFLSFADYFRPRYFLLENVRTFVSFNKGQTFQLTLASLLEMGYQVRFGILEAGAYGVSQSRKRAFIWAAAPEEVLPEWPEPMHVFGVPKLKISLSQGLHYAAVRSTALGAPFRPITVRDTIGDLPSVENGDSRTNKEYKEVAVSWFQKEIRGNTIALTDHICKAMNELNLIRCKLIPTRPGADWHDLPKRKVTLSDGRVEEMIPFCLPNTAERHNGWKGLYGRLDWQGNFPTSVTDPQPMGKVGMCFHPEQHRILTVRECARSQGFPDSYEFAGNINHKHRQIGNAVPPPLAFALGRKLKEALHLKKSPQHQP

SEQ ID NO: 122 - DNA, 4695 bp, Prunus persica
  misc_feature (1)..(4695): Public GI no. 37039880
atgggttccgcagcggcagcagaagcggcagaagcagcagcgctcttggaggccaaaggtgccaatgggactaaaccaccatcttcgtcatcttcaggaatgacgaagaagaagaagggtaaacaagattcccaaaaggcagcacctaaagctaagaagcgaaatttgcctcagagcagtgaagaagagccttcccgatctcggaaaatgccgaagcgggctgctgcttgcaaagactttaaggataggtctgttcatatttctgagaagtctagccttattgaaagcaaggaggaccagatagtggaggaagaaattcttgccgtacgcctgacttgtggcccggaccaagatgctgtgcgcccaaacagaagactgactgattttgttttgcatgatgcaactggttccgcacaaccccttgagatgttggaagtttctgacatgtttatatctggtgctatattgcctctcaatgaaagttctgacaaggacaagggaagaagtgttagatgtgaaggtttcgggcggatagaatcttgggacatctctggttatgaagatggctcccccgtaatatggctttcaactgaagttgctgattatgattgccgtaaaccggccagtagctacaagaaatactttgatcaattctttgagaaagcgcgtgcttgcatagaggtttacaagaagctgtctaaatccaactccgacaactccgaccccactcttgatgaattgcttgctggtattgcacgatcaatgagcgggagcaaattcttttctgggagtgcatctgtcaaagactttgttctatctcaaggcgagtttatttatgctcaagtaataggtctggaggaaacatcaaagaagaacgatcggccatttgcagagttacctgtccttgctgccctcagagatgagagtataaagcgtggaaattttgtgcaatcaaaaccgggaatttcaagtggtactttaaagattggtggagagaacggagtggattcagctggttcatccgtagttgaagctgaggaaaatgaggatgcaaagttggcaaaactcttgcaagaggaagaatactggaagtcaatgaaacaaagaaagcgccagggtcctgcctctgtgtcaagcaaatactacatcaaaattaatgaagatgaaattgccaatgattatcctctacccgcttattacaagaattgcattgaagaaactgatgagttcatagtttttgacaatgagtttgatatctgtaatgctgatgaccttcctcgaagtatgcttcataattggtgtctatacaactcggactcaagattgatttcgctcgagcttcttccaatgaaaccctgcgcagacattgatgttaccattttcgggtcaggggttatgagtgaagatgatggaagtggcttttgtcttgattctgatggtacttcaagtggtccaggagcccaggatgctgatggaatgccaatttacttgagtgcgataaaggaatggatgattgaattgggagcatcaatggtttcaatatcaatccgaacagatatggcctggtacagacttggcaagccatctaagcagtatgctctgtggtatgaaccaattctgagaacagcaaagattgggagaagtataatcactatgctgaaagatcaaagtcgagtagcacggctttctttcgcagatgtcattaagagactgtcagggttccaaaaggaccattgtgcttacatttcttctgatccagcatttgttgagaagtatgtcgttgtccatggacagataatactgcaactgttttcagaatttccagatgcgcagattaaaaaatgtccatttgtgattggtcttacaaagaaaatggaggagaggcaccatactaaatggttagtaaagaagaagaagcttgtggaaaagagtgaatcaaatttgaacccaagggcatcaatggcacctgtggtttccaagaggaagacaatgcaagctacaacaacaaggctgatcaacagaatctggggggagtactattcaaactactctccagaagattcgaaggagggagatattggagaaaagaaagaggaggaggaagttgaagaagaggatgtagaagaggatgatgtagaagagaatccaactgtaatggagcaagcccagaagccttcttcaatttcaagacaaaccaaatcatgcctcaacaacagggaaattttgtgggaaggggagccagtgggccaaacatgttctggtgaagctctttataagcgtgccattctttggggagaagaaatttctgttggcggtgctgttttggtggaacttgatgaatcccatgaacttcctgccatttattttgtggagtatatgtatgaaacattgaatggaagcaaaatgtttcatggaagagtgatggagcgaggatcccagactgttcttggcaacactgccaatgagagggaggtatttttgacaaatgagtgcacaaatttggcattaaaggaagttaaacaggcagctgctgtgggcattaaagtaatgccgtgggggcatcagtataggaaggataatgctgatgctaacagaactgatagagcaagggcagaagagaggaagaggaagggtttgccgactgaatattactgtaaaagcttgtattgcccagagagaggtgctttccttagtctttcacgtgatactatgggtctgggttctggtgcctgccactcttgcaaaatgaatgaagccgaggaggccaaggaagtatttaaagtgaattcatcaaaaactggttttgtatacaggggagttgagtactcagttcatgattatgtctatgtaagtccccattattttggtgtggaaaggatggaaactgaaattttcaaggctggaaggaatttggtgctgaaagcttatgtcgtgtgccaagtgctggagatagttgttatgaaggagtctaaacgacctgaaatagaatctacccaggttaaagtaagaagatttttcagaccagaggacatatctgttgagaaggcatacagttcggatattagagaggtctactacagtgaacaaacacacatcgtgcctgttgataatatagaaagaaaatgtgaagtcagaaagaagagtgatcttccagtatgtaatgctcctgtcattttccagcatattttcttctgtgaacatctatatgatccttctaaagggtctattaagcagttgccagctcacatcaaactgaggtactcaacaggaggtgggcatgctgattctagaaagagaaagggcaagtgcaaagaaggagaaaatgtttcagaagttgagaaccagagagttgattctgagcagaaacgcctagccacattggatatatttgctggttgcggtggcttgtctaatgggttgcgtcagtctggtgcttcaataaccaagtgggcaattgagtatgaagagcctgctggggatgctttcaaactcaaccatcctgagtcattggtttttatcaataactgcaatgtgatcttaagggccgtaatggaaaaatgtggggacacagatgattgtattgcaacttctgaagctgctgaattggctgcatcacttgatgaggaggttaaaaatgatttgccgttgccggggcaggtagatttcatcaatggaggacctccatgccggggtttctctggaatgaataggttcacccaaagcccttggattaaatttcattgtaaaatgatttgggcttgcttagcctttgccgactacttccggccaaagttgttcccgctggagaatgtgaggaaatttgtgtcattcaataaagggcagacatttcagcttactttggcttcccttctcgaaatgggttaccaggtgagatttggaatcctggaggccggtgcatatggaatatcccaatctcgtaaacgagctttcatttgggctgctgcaccagaagaagttctccctgaatggcctgagccgatgcatgtctttggtgttccaaagttgaaaatctcactatctcaaggtttacattatgctgctgttcgtagtactgcacttggtgcccctttccgtccaatcaccgtgagagacacaattggtgatcttccatcagtagaaaacggagactctaggacaaacaaagagtataaagaggttgcagtctcgtggttccaaaaggagataagaggaaacacgattgctctcactgatcatatctgcaaggctatgaatgagcttaacctcattcgatgcaaattaatcccaactaggcctggggctgattggcatgacttgccaaagagaaaggttacgttatctgatgggcgcgtagaagaaatgactcctttttgtctcccaaacacagctgagcgccacaacggttggaagggactatatgggagattagattggcaaggaaactttccgacttccgtcacggatcctcagcccatgggtaaggttggaatgtgctttcatcttgaacagcacagaatccttacagtccgtgaatgcgcccgttctcaggggtttccggatagctacgagtttgcagggaacataaatcacaagcacaggcagattgggaatgcagttcctcctactttggcctatgcattggggactaaactcaaggaagcaattgacagcaagaggttgtcttcacaagagtaa

SEQ ID NO: 123 - protein, 1564 aa, Prunus persica
  misc_feature (1)..(1564): Public GI no. 37039880
MGSAAAAEAAEAAALLEAKGANGTKPPSSSSSGMTKKKKGKQDSQKAAPKAKKRNLPQSSEEEPSRSRKMPKRAAACKDFKDRSVHISEKSSLIESKEDQIVEEEILAVRLTCGPDQDAVRPNRRLTDFVLHDATGSAQPLEMLEVSDMFISGAILPLNESSDKDKGRSVRCEGFGRIESWDISGYEDGSPVIWLSTEVADYDCRKPASSYKKYFDQFFEKARACIEVYKKLSKSNSDNSDPTLDELLAGIARSMSGSKFFSGSASVKDFVLSQGEFIYAQVIGLEETSKKNDRPFAELPVLAALRDESIKRGNFVQSKPGISSGTLKIGGENGVDSAGSSVVEAEENEDAKLAKLLQEEEYWKSMKQRKRQGPASVSSKYYIKINEDEIANDYPLPAYYKNCIEETDEFIVFDNEFDICNADDLPRSMLHNWCLYNSDSRLISLELLPMKPCADIDVTIFGSGVMSEDDGSGFCLDSDGTSSGPGAQDADGMPIYLSAIKEWMIELGASMVSISIRTDMAWYRLGKPSKQYALWYEPILRTAKIGRSIITMLKDQSRVARLSFADVIKRLSGFQKDHCAYISSDPAFVEKYVVVHGQIILQLFSEFPDAQIKKCPFVIGLTKKMEERHHTKWLVKKKKLVEKSESNLNPRASMAPVVSKRKTMQATTTRLINRIWGEYYSNYSPEDSKEGDIGEKKEEEEVEEEDVEEDDVEENPTVMEQAQKPSSISRQTKSCLNNREILWEGEPVGQTCSGEALYKRAILWGEEISVGGAVLVELDESHELPAIYFVEYMYETLNGSKMFHGRVMERGSQTVLGNTANEREVFLTNECTNLALKEVKQAAAVGIKVMPWGHQYRKDNADANRTDRARAEERKRKGLPTEYYCKSLYCPERGAFLSLSRDTMGLGSGACHSCKMNEAEEAKEVFKVNSSKTGFVYRGVEYSVHDYVYVSPHYFGVERMETEIFKAGRNLVLKAYVVCQVLEIVVMKESKRPEIESTQVKVRRFFRPEDISVEKAYSSDIREVYYSEQTHIVPVDNIERKCEVRKKSDLPVCNAPVIFQHIFFCEHLYDPSKGSIKQLPAHIKLRYSTGGGHADSRKRKGKCKEGENVSEVENQRVDSEQKRLATLDIFAGCGGLSNGLRQSGASITKWAIEYEEPAGDAFKLNHPESLVFINNCNVILRAVMEKCGDTDDCIATSEAAELAASLDEEVKNDLPLPGQVDFINGGPPCRGFSGMNRFTQSPWIKFHCKMIWACLAFADYFRPKLFPLENVRKFVSFNKGQTFQLTLASLLEMGYQVRFGILEAGAYGISQSRKRAFIWAAAPEEVLPEWPEPMHVFGVPKLKISLSQGLHYAAVRSTALGAPFRPITVRDTIGDLPSVENGDSRTNKEYKEVAVSWFQKEIRGNTIALTDHICKAMNELNLIRCKLIPTRPGADWHDLPKRKVTLSDGRVEEMTPFCLPNTAERHNGWKGLYGRLDWQGNFPTSVTDPQPMGKVGMCFHLEQHRILTVRECARSQGFPDSYEFAGNINHKHRQIGNAVPPTLAYALGTKLKEAIDSKRLSSQE

SEQ ID NO: 124 - DNA, 5286 bp, Daucus carota
  misc_feature (1)..(5286): Public GI no. 2895089
atgcagagaagagaaatataccaggagttgaagagaagagaaataaacgaaactgcagtttgtgattctgagaagggatctgcaagtggcaaaaagaaagtgaaagcacacaacttgcaaagtagtgaggtttctgccggctctcctaaaatcctaaagctagctgctgcttgtgctgatgacaaagagaaggctgacaaagtatctaaagaaatatcaagcactaaaacaagaaaagatcagggtgtagatgaagaagtcgctgtgcaaaaagcacatgcctcacagagtagtgaggttcctgctggttcccgaaaaatgccaaagcgagctgctgcttgtgctgatgataaagagaaagctgtgaaaatatccaaaaaggtatcaagcagtaaaacaaaaaagggtcagtgcgtagaagtagctgctgagcagaaagcacaagtctcaaagagtagcgacgtttctgctggttctcggaatatgccaaagcaagctgctggttgtgctaatgacaaagtgaaagctgtgaaaatatccagaaaaagcactaaagcaaaaaaggatcagggtttagatgaagaggaagtggctgtgaaagaggcacatgtctcacagagcagtgaggtttctgctggttcccgaaaaatgccaaagcgagctgctgattgtgctgatgataaagagaaagctgtgaaaatatccaaaaaggtatcaagcgctaaagcaaaaatggatcaggctttagacgaagaggaagtggctgtacaagaagcacatgtctcaaagagtagcgaggtatctgctggttcccggaaaatgccaaagcgagctgctgcatgtgcagatttcaaagagaaagttgttcaaatatccaaaaaagcatcaattatcgaaacgaaaaaagaccggtgtgtagatgaggaggaaatggctgtgcggctaacagctggacaagaagatggccggccatgcaggagactcactgattttattttacacaattctgatggggtacaacaaccctttgaaatgttggaagttgatgatttgtttatctctggcctgattttgcctcttgaagaaagttcccaaaaagaggattgtagcatcagatgtgaaggctttggacgaattgaagactgggccatttctggatatgaagacggggttccaataatatgggtttcaacggatgttgcagattatgattgtgtcaaaccatcagctgcttacaagaagcactatgaacacttttttgccaaagccactgcttgtattgaagtgtataagaaattgtcaaaatcttctggaggaaatcctgatttgagctttgatgagttgcttgctggggttgttcgtgcaatgaatggtatgaagtgcttctctcgtggggtatccattaaagatttcatcatttctcagggtgagtttatctacaatcaacttgttgggttggacgagacatctaaggatgatcagcaatttctcgagctacctgtcctcgtagccctaagagatgaaagtagcaggcatgtaaatgattttcaagaaaggattggatgcacaaatggaacactaaagattagagacaatgaggaccaaaagaattctgtaacagaggaaggagaggataaaaaaatggcaagattgctgcaggaagaagagttctggaagtcaatgaagcagaagaaaggtcagggttcaagggtggcttctaccaaatattatataaaaataaatgaggatgagattgcaaatgactatcctctgcctgcatattacaagacagccaaccaagaaacagatgaatacataattttcgatggaggccttgatgcatgttatactgatgatttgcctcgaagtatgcttcataactgggcattatacaactcagattcaaggctaatttccttggaacttcttccaatgaaaccttgtgctgaaattgatgtaactatatttggatcaggggtgatgactgaggatgatggatctgggtttaatcttgaaaccgacacatctcattcttcctctagtggatcaggaacagcaaatgttgatggcattcctatatacttgagtgccataaaggaatggatgatcgaatttggatcctcaatggtatttatatcgatacgcacagatatggcctggtataggcttggcaagccatcaaagcagtatgcaccatggtatgaaccagttcttaaaactgccagggttgcaatatccattatcacattattaatggagcaggcccgagtttctcgtctttcatttatggatgttatcaaaagagtttcagagtttgagaagggtcatcctgcttacatttcatctattccagcagttgttgaaagatatattgttgtgcatggacaaattatcctgcagcagttcttagaatttcctgatgagaagattaaaaagtctgcatttgtggccggcctcacaaaaaaaatggaagaaaggcatcacaccaagtggcttgtgaagaagaaaaagattttgcagagggatgaaccaaatttaaatcctagagcagcaatagcccctgtggtatctaagaggaaggctatgcaggcaacaacaacaaggctaattaacaggatctggggtgagttttattcaaactactctccggaagatatgaaagagggaataactagtgatgaaaaggaggacgaagaagctgaagagcaagaggaaattgatgatgaggaggaagatgaggagaaggaaactttagtggctttagaaaaaactcccacacccacctcaacaccaagaaaatcgaaatcgaactctaaactgaaggacgtaagctggaatgggaagcctgcggtaaaaagatcatctggtgagatgctgtataaacaggcaacactacatggaaacatgattgctgtcgggggggctgttttgacagatgatgcatcttgcctgaaccttccagccatctattacgtggagtacatgtttgaaagttcagatggaaaaatgattcatggaagattgttgcgacaaggatctgaaacggttcttggaaatacggctaatgaacaggagttatttcttacaaatgaatgcatggaatttgaactgatggatgttaagatgcctgttattgtggaaattcggtcaaggccttggggacaccagcacagaaaaataaatgcaaacgctgacaagattgataaggccagagctgtagaaaggaaaaataagggattggaaactgaatactactgcaaaagtttgtattggccagagagaggtgcttttttcagtcttccagttaattgcatgggtttgggttctggtatctgtagttcttgcagtgcaaataaggaccacactgaaaaagaaaaattttctgtgagctcatgcaagacaagttttgtgtacaagggaactgagtattccgttcatgattttctctatgtaagtcctgatcagtttgctacagaaagagtagggcaggaaaccttcaaaggtgggcgaaatgtgggattaaaagcttttgctatatgtcaattgcttgaagttgtggtccctaaaaaaccccagcaagctgatgacagttctactgaggtcaaggtaaggagattttaccgaccagaagacatttcagatgagaaggcttattgctcagacattcgagaggtttattatagcgaagaaacacacacccttcttgtcgaggcaattgaagggagatgcgaagtgagaaagaaaagtgaccttccaacatgtgatgcccccactatatatgagcatgttttctattgtgaatatctctatgatcctcataaaggttctctaaagcagttgccatcgaacatcaagttgaggtattcaactgttaagggtgcatatgattcttccttaagaaagaacaagggaaaatgtaaggaaggggaggatgatttagaagctgagaaatcaaaagagaactgtttggctacgttggatatctttgctggttgtggaggcctgtcagaagggttgcagcagtctggtgtctgtagaacgaagtgggcaattgaatatgaagagcctgcgggagatgcgttcaaactcaatcatccagacaccacaatgttcatcaataattgcaatgtgattctaaaggccatcatggataagagtggagatgcagatgattgcatttcaacaccagaggcagcagatttagctgctaaattaagtgaggaggaactaaagaatttgccactgccaggacaggttgattttatcaatggaggccccccttgtcagggattctctggaatgaatagatttaaccagagttcttggagtaaagtacaatgcgagatgattttagcattcttatcctttgcggattattatcgaccaaagtattttcttcttgagaatgtgaggaattttgtgtccttcaacaagggacaaacattccgtctagctattgcttcacttcttgagatgggttaccaggttcggtttggtatacttgaagctggagcatttggagttcctcaatcgaggaagcgtgcatttatatgggcagcatctcctgaagaaactctccccggaagttggccagagccgatgcatgtctttgctgcaccagagctaaaggttgcattacctggaaacaagcattatgctgctgtccggagtactcaagctggagcaccgtttagagctatcactgttagggatactataggagatcttccgatggttaccaatggggcgtccaagacaactttagagtatcgatgtgatcccatctcatggtttcaaaagaatatccgggcaaatatgatggtcctgacagatcacatatcgaaagaaatgaatgagctcaatctcattcgctgtcagagaattccaaagcgacgaggtgcagattggcatgaccttcctgaggaaaaggtcaaactgtcaactgggcaattagtagacttgataccatggtgccttccaaatactgctaagaggcataaccaatggaaagggctatttggaaggttagattgggaaggaaactttccgacttctatcactgatcctcaaccaatgggaaaagttgggatgtgctttcatcctgatcaagacaggattgtaacagtccgagagtgtgcacgctcccaaggctttccagatagctatcagttttatggcaacattctacacaagcaccgacaaattggaaatgcagttcctcctcctttagcatatgcacttgggaggaaactcaaagaagctttggagagcaagggctcaatgtaa

SEQ ID NO: 125 - protein, 1761 aa, Daucus ca rota
  misc_feature (1)..(1761): Public GI no. 2895089
MQRREIYQELKRREINETAVCDSEKGSASGKKKVKAHNLQSSEVSAGSPKILKLAAACADDKEKADKVSKEISSTKTRKDQGVDEEVAVQKAHASQSSEVPAGSRKMPKRAAACADDKEKAVKISKKVSSSKTKKGQCVEVAAEQKAQVSKSSDVSAGSRNMPKQAAGCANDKVKAVKISRKSTKAKKDQGLDEEEVAVKEAHVSQSSEVSAGSRKMPKRAADCADDKEKAVKISKKVSSAKAKMDQALDEEEVAVQEAHVSKSSEVSAGSRKMPKRAAACADFKEKVVQISKKASIIETKKDRCVDEEEMAVRLTAGQEDGRPCRRLTDFILHNSDGVQQPFEMLEVDDLFISGLILPLEESSQKEDCSIRCEGFGRIEDWAISGYEDGVPIIWVSTDVADYDCVKPSAAYKKHYEHFFAKATACIEVYKKLSKSSGGNPDLSFDELLAGVVRAMNGMKCFSRGVSIKDFIISQGEFIYNQLVGLDETSKDDQQFLELPVLVALRDESSRHVNDFQERIGCTNGTLKIRDNEDQKNSVTEEGEDKKMARLLQEEEFWKSMKQKKGQGSRVASTKYYIKINEDEIANDYPLPAYYKTANQETDEYIIFDGGLDACYTDDLPRSMLHNWALYNSDSRLISLELLPMKPCAEIDVTIFGSGVMTEDDGSGFNLETDTSHSSSSGSGTANVDGIPIYLSAIKEWMIEFGSSMVFISIRTDMAWYRLGKPSKQYAPWYEPVLKTARVAISIITLLMEQARVSRLSFMDVIKRVSEFEKGHPAYISSIPAVVERYIVVHGQIILQQFLEFPDEKIKKSAFVAGLTKKMEERHHTKWLVKKKKILQRDEPNLNPRAAIAPVVSKRKAMQATTTRLINRIWGEFYSNYSPEDMKEGITSDEKEDEEAEEQEEIDDEEEDEEKETLVALEKTPTPTSTPRKSKSNSKLKDVSWNGKPAVKRSSGEMLYKQATLHGNMIAVGGAVLTDDASCLNLPAIYYVEYMFESSDGKMIHGRLLRQGSETVLGNTANEQELFLTNECMEFELMDVKMPVIVEIRSRPWGHQHRKINANADKIDKARAVERKNKGLETEYYCKSLYWPERGAFFSLPVNCMGLGSGICSSCSANKDHTEKEKFSVSSCKTSFVYKGTEYSVHDFLYVSPDQFATERVGQETFKGGRNVGLKAFAICQLLEVVVPKKPQQADDSSTEVKVRRFYRPEDISDEKAYCSDIREVYYSEETHTLLVEAIEGRCEVRKKSDLPTCDAPTIYEHVFYCEYLYDPHKGSLKQLPSNIKLRYSTVKGAYDSSLRKNKGKCKEGEDDLEAEKSKENCLATLDIFAGCGGLSEGLQQSGVCRTKWAIEYEEPAGDAFKLNHPDTTMFINNCNVILKAIMDKSGDADDCISTPEAADLAAKLSEEELKNLPLPGQVDFINGGPPCQGFSGMNRFNQSSWSKVQCEMILAFLSFADYYRPKYFLLENVRNFVSFNKGQTFRLAIASLLEMGYQVRFGILEAGAFGVPQSRKRAFIWAASPEETLPGSWPEPMHVFAAPELKVALPGNKHYAAVRSTQAGAPFRAITVRDTIGDLPMVTNGASKTTLEYRCDPISWFQKNIRANMMVLTDHISKEMNELNLIRCQRIPKRRGADWHDLPEEKVKLSTGQLVDLIPWCLPNTAKRHNQWKGLFGRLDWEGNFPTSITDPQPMGKVGMCFHPDQDRIVTVRECARSQGFPDSYQFYGNILHKHRQIGNAVPPPLAYALGRKLKEALESKGSM

SEQ ID NO: 126 - DNA, 4680 bp, Lycopersicon esculentum
  misc_feature (1)..(4680): Public GI no. 2887280
atggcgtcaccccaacctaattcggagtcggtattagaacttccgaacaacgacaaatctggacacaaaaagaacaaacgcaaacaagattctgtgtcaaaaaggaaggcatctgcaactggtaagaaggaaaagaaacaggctgtttctgaaactattgaggagcccactgctggacgtaaaaggcctaagcgagctgctgcctgttcagattttaaagagaaatctgtgcatttatcaaaaaagtcttcagtcattgaaacaaagaaggaccattgtgtagacgaagaggatgtagctattaggttaactgcgggtctgcaagagtctcaacgaccctgtagaagattaacggattttgtttttcataactcagaaggaataccacaaccgtttggaatgtctgaggttgatgatctgtttatcagtggcctcattttaccacttgaggacagtcttgacaaagtaaaagcaaaaggaattagatgtgaaggctttgggcgtattgaagaatgggctatctctggctatgaagatggaactcctgtcatatggatctcaactgagacagctgattatgattgtttaaaaccctcaggtagttataagaagttttatgaccacttcttggccaaggcgacggcttgcgttgaggtttataagaagctttcaaagtcatctggagggaatcctgatttaagtcttgacgagttgcttgcaggggttgtccgagcgatgactggcataaaatgcttttcaggtggagtatccatcagggactttgtcatcactcagggcgggttcatatataaggaacttattggtctggatgatacatcaaagaagactgatcaactttttgttgagctacctgtcctagcttcccttagagatgaaagcagcaagcacgagacacttgcacaaccagagactatatcatctggtaatggtctacgtattggcccaaaagcaggaaatggaggagacaagatagttgaatctggtttggccaatggtccagcgccagaagatgaagatctaaaattggctaaattgttgcatgaagaggagtattggtgctccttgaagcagaagaaagaccgtaatacatcttcctcatccagcaaaatatacatcaagatcaatgaggatgagattgcaagtgattatcctttacctgcatattacaaaacatctaatgaagagactgatgagtatattgtctttgacagtggggttgaaacataccatattgatgagttgcctcgcagcatgcttcataattgggcattatacaactcggactcaaggctaatatctttagaactgctgccaatgaaagcttgtgctgatattgatgtaaccatttttgggtctggagtgatgactgctgatgatgggtctggctacaattttgacacagatgctaatcattcctcttcaggtggttctagatcagctgaaattgatggaatgccaatttacctgagtgctataaaagaatggatgattgagtttgggtcctcaatgatctttatatcaattcggactgatatggcctggtataggcttgggaagccattgaaacagtatgctccttggtacgaaccagtcataaagactgcaagattggcagtgagcatcattactttgttaaaggaacagaatcgtgtggctagactttcttttggagaagttattaaaagggtttcagagttcaagaaagaccatcctgcttatatatcatctaatgtagatgcagtggaaaggtatgtggttgtacatgggcaaattattctccagcagttttctgaatttcctgatgtaagcattaggaattgtgcatttgcggttggtctctcaaggaaaatggaagagaggcaccatacaaaatgggtgattaagaagaagaaggtgatgcagagactggaacagaacttaaatcctagagcatctatggcgccatctgtaaaaaggaaagctatgcaggctactacaacaaggctaatcaacagaatctggggggaatactattccaattactcacccgaggtgtcaaaggaggtggctgattgtgaggtgaaggatgatgaagaaccagatgagcaagaggaaaatgaagaggatgatgttccggagaggaacttggatgttccagagaaagctcatacaccttcttctacaagaaggcatattaagtcacgttctgacagcaaagaaataaactgggatggggaatccataggtaaaacagcttctggtgaacagttgtttaaaaaagctagagttcatggacatgagatagctgttggagattcagttctagtggaacatgatgaaccagatgagcttggttgtatttactttgttgaatacatgtttgaaaaattggatggtagcaaaatgcttcatggaaaaatgatgcaacgaggatctgacactgtacttggaaatgcagctaatgagagagaggtatttttgatcaatgaatgcatgaatctgcaactaggagatgtcaaagaaagtatagctgtcaatatcagaatgatgccttggggacaccagcatagaaacacgaatgctgataaacttgaaacagcaaaagcagaagacagaaagaggaagggattgccgacggaattttactgcaaaagcttttatcgccctgaaaaaggtgcttttttcagactcccgtttgataagatgggccttggtaatggtttatgctactcttgtgagttgcagcaaactgatcaggaaaaggaatcctttaagtttgatatgtccaaatccagttttgtatatctggggactgagtattcagttgatgactttgtttatgtaagccccgatcactttactgcagaaagagggggaaatggaactttcaaagccggaagaaatgtggggttgatggcctatgtagtatgtcaattactagaaattgttggacctaagggatctaaacaagctaaagtagattctacaaatgttaaagtcagaagattcttcagaccagaggatatatcttcagataaggcatactcttctgatatccgggagatctattacagtgaagatatacatacagttcctgtggaaataatcaaaggaaaatgtgaagtgaggaagaagtatgatatttcctctgaagatgtccctgccatgttcgaccatattttcttttgtgaatatttgtatgatccattgaatggatcccttaagaagttaccagctcagataaacctgatattgtcaaaaattaagctagatgacgcaacatctaggaagaggaaggggaagggaaaagaaggagtggatgaagttggggaactaaatgaaacttctccacagaatcgtttgtccacattagatatctttgctggttgtggtggcttgtctgaggggttgcagcattcgggtgtcacagatacaaattgggcaattgaatacgaagcgcctgctggagatgcatttagacttaatcatccaaagacaaaggtgttcatacataattgcaatgtgattttgagggctgtcatgcagaagtgtggagattctgatgactgtatctcaactccagaggcttctgaattagctgcagcaatggatgagagcgaactgaatagtttgccactgcctggacaagttgatttcattaatggaggccctccttgtcaggggttttctggaatgaatagatttaatcagagcacctggagtaaagtacagtgtgagatgattctggcatttttatcctttgctgattattatcggcccaagttttttctcttggagaatgttaggaattttgtttcgttcaaccaaaaacaaacatttcgcttaactgttgcttcccttcttgagatgggttatcaggttaggtttggtatccttgaagccggagcgtatggagttcctcagtctaggaagagagcatttatctgggctggctccccagaggaggttcttccagagtggccagaaccaatgcatgtttttgctgtcccagaattaaaaatcgcattatctgaaacttcatactatgcagctgtgaggagtactgctagtggagctccattccgttcacttactgtcagagacacaattggagatcttcctgttgttggcaatggggcaagcaagacttgcatagagtatcaaggtgatccagtatcctggttccaaaagaaaatccggggcagctcaataacattatctgatcacatttcaaaagagatgaatgagcttaacctaatcaggtgccaaagaatccccaagcggccaggagctgattggcgtgaccttgaagatgaaaaggttaaactatctaatggtcaactagttgatttgattccatggtgcctgcctaacactgctaagcggcacaaccagtggaaggggctctttggaaggttggattgggatgggaacttccccacttctattactgatccccagccgatgggcaaggtggggatgtgctttcatccagatcaagacaggattgttacagttcgtgaatgtgcacgttctcaaggtttcccagacagctaccaatttgctggtaacatcttgcacaagcacaggcaaataggaaatgctgttccacctcctttggcatatgcgcttggaagaaaactcaaagaagctgttgagagcaaaaataggctcacttag

SEQ ID NO: 127 - protein, 1559 aa, Lycopersicon esculentum
  misc_feature (1)..(1559): Public GI no. 2887280
MASPQPNSESVLELPNNDKSGHKKNKRKQDSVSKRKASATGKKEKKQAVSETIEEPTAGRKRPKRAAACSDFKEKSVHLSKKSSVIETKKDHCVDEEDVAIRLTAGLQESQRPCRRLTDFVFHNSEGIPQPFGMSEVDDLFISGLILPLEDSLDKVKAKGIRCEGFGRIEEWAISGYEDGTPVIWISTETADYDCLKPSGSYKKFYDHFLAKATACVEVYKKLSKSSGGNPDLSLDELLAGVVRAMTGIKCFSGGVSIRDFVITQGGFIYKELIGLDDTSKKTDQLFVELPVLASLRDESSKHETLAQPETISSGNGLRIGPKAGNGGDKIVESGLANGPAPEDEDLKLAKLLHEEEYWCSLKQKKDRNTSSSSSKIYIKINEDEIASDYPLPAYYKTSNEETDEYIVFDSGVETYHIDELPRSMLHNWALYNSDSRLISLELLPMKACADIDVTIFGSGVMTADDGSGYNFDTDANHSSSGGSRSAEIDGMPIYLSAIKEWMIEFGSSMIFISIRTDMAWYRLGKPLKQYAPWYEPVIKTARLAVSIITLLKEQNRVARLSFGEVIKRVSEFKKDHPAYISSNVDAVERYVVVHGQIILQQFSEFPDVSIRNCAFAVGLSRKMEERHHTKWVIKKKKVMQRLEQNLNPRASMAPSVKRKAMQATTTRLINRIWGEYYSNYSPEVSKEVADCEVKDDEEPDEQEENEEDDVPERNLDVPEKAHTPSSTRRHIKSRSDSKEINWDGESIGKTASGEQLFKKARVHGHEIAVGDSVLVEHDEPDELGCIYFVEYMFEKLDGSKMLHGKMMQRGSDTVLGNAANEREVFLINECMNLQLGDVKESIAVNIRMMPWGHQHRNTNADKLETAKAEDRKRKGLPTEFYCKSFYRPEKGAFFRLPFDKMGLGNGLCYSCELQQTDQEKESFKFDMSKSSFVYLGTEYSVDDFVYVSPDHFTAERGGNGTFKAGRNVGLMAYVVCQLLEIVGPKGSKQAKVDSTNVKVRRFFRPEDISSDKAYSSDIREIYYSEDIHTVPVEIIKGKCEVRKKYDISSEDVPAMFDHIFFCEYLYDPLNGSLKKLPAQINLILSKIKLDDATSRKRKGKGKEGVDEVGELNETSPQNRLSTLDIFAGCGGLSEGLQHSGVTDTNWAIEYEAPAGDAFRLNHPKTKVFIHNCNVILRAVMQKCGDSDDCISTPEASELAAAMDESELNSLPLPGQVDFINGGPPCQGFSGMNRFNQSTWSKVQCEMILAFLSFADYYRPKFFLLENVRNFVSFNQKQTFRLTVASLLEMGYQVRFGILEAGAYGVPQSRKRAFIWAGSPEEVLPEWPEPMHVFAVPELKIALSETSYYAAVRSTASGAPFRSLTVRDTIGDLPVVGNGASKTCIEYQGDPVSWFQKKIRGSSITLSDHISKEMNELNLIRCQRIPKRPGADWRDLEDEKVKLSNGQLVDLIPWCLPNTAKRHNQWKGLFGRLDWDGNFPTSITDPQPMGKVGMCFHPDQDRIVTVRECARSQGFPDSYQFAGNILHKHRQIGNAVPPPLAYALGRKLKEAVESKNRLT

SEQ ID NO: 128 - DNA, 4650 bp, Populus balsamifera subsp. trichocarpa
  misc_feature (1)..(4650): Ceres GDNA ANNOT ID no. 1478748
atgaggaagaacaaaaaagggaaacaaaaatcttcagtgtctaatgtaaagaaagaagtgccagaaaaaaacacaaaagggaagaagagaaattcccctgataccaacaaggaagagcctactggtggcgatggttcactaaaaaggcctaagagagctgctgcatgcaaagatttcaaggagaaatctgtacgattgcatgaagagaagtcttatgttgttgaatcaaagaaagaacaagttgtcgatgaagaaattctagctgttcgtttgactcaagggcaagaagagggtcgcccaaacaggagattgattgattttgttgtgcatgatgcaaatggcaacccacagcccttggagatggttgaagttgatgatatgtttatctctggcattataatgcctcacgaggaaagccttgacaaggaaaaagaagtgcatgttaggtgtgatggctttggaaggatagaagcatgggatatctctggttatgaagatggatccccagtcatttggcttagtactgaagtggcagattatgactgcattaagcctgctggtggctacaagaagttctttgatcatttcttccagaaggcacttgcttgtgttgaagtctacaaaaaactctcaagattctctggaggaaaccctgaattcacccttgatgagttgcttgctggggttgtgcgagcaatgagtggaaataagtgtttctctggtgcagtatccatcaagaatttcctcatttctcagggtgaatttatttatcaccaaataattggtttggatgagacttccacaaagaatgataagaaatttgcagacttgcctgttcttgtagccttgagagatgaaagtcgcaatcatggaaatgttctgatagcaaaagcagcaaattctggtgggaacttggtgattggtccagaatctgtggatggtgctgttgtgaatcagtctaaccaatccagtaccacagttgaggaagacgaggatgcaaagttagcaagattattgcaggatgaagagtattggcagtctaatatgaggcagaagaaaagtcgtggttcagtttctgcatccaacacaatctacatcaaaattaatgaagatgagattgcaaatgactaccctctccccgcgttttacaaacactctaatgaagaaaccgatgagtatatagctgttgcaagtgatgatgtaattgaccaccctgatgatctccctagaagaatgcttcataactggtcactttataactcagactcaaggttgatttctttggagcttcttccaatgaaaccttgtgaagacattgatgtcaccatctttggatcaggaagtatgactgaagatgacggaagcggattctgtcttgatgatggtcctgatcagtcttcttcaaggggtttggaggctcaggatgacatgggtttgccgatatttttgagtgcgataaaggagtggatgattgagtttggatcatcaatgatttttatatcactacgcacggacatggcctggtatagacttggaaagccatcaaaacaatatgcttcctggtataagccagtcctaaaaacagttaagcttgcaagaaccatcattacattgttgaaggagcaaagtcgagtatcacggctttcttttgcagatgtaatccggaaagtgtctgagttcaagaaggatcatcatgcttatatttcttctgatctagcagctgttgagaggtacgtggtagtgcatggacagataatactacagctttttgctgagtttccagatcagaagatcaagaaatgtgcttttgtggtcggtttgactcgtatgatggaggagaggcaccatactaaatgggtagtgaacaaaaaagcaattgtgcaaaagtgtcattccaatttgaatcctcgggctgcaatggatactgttgcttctggagcgtccaagaggaagcttatgcaggcaacaacaacaagactgatcaacagaatatggggggaatattattcaaactattctccagaggatttgaaagagggaaatgattgtgatgtaaaagaagaagatgaacttgaggaacaggatgaaaatgaagatgatgataaagaggtggtggtagagaaaacactaaaaccttattcagtgtttgaacattgtaaatcccatacttcacaaaaagaggtaagatgggatgggaatcctgtaagaaaaatatcttctggagaagacatttacaagcaagccattgtttgtggacaggtcattgtggtgggggctgctgtcttagtggaggttgatgaaccagatgaacttcctgccatttattttgtggagtacatgtttgaaacaagaaatggaagtaaaatgtttcacgggagaatgatgaagtggggatctgagacagttcttggtaacactgccaatgatagagaggttttcttaacaaatgaatgcatgaattataaactacaggatgttaagcaaacaataattctggaagttcgaaaaaggccttggggacatcatcacaggaaagataatgccaatgctgatagaattgatagggaaaaggcagaagagaggaaaaagaaaggattgccactagaatattactgtaaaagcttgtactggccagaaaggggtgctttcttcactcttccatttgatactatgggtcttggctctggtgtctgccactcttgtaacttaaaaatatctgaagaagacaagaatatttccaaagtgaattcttctcaaacaggattttcatacaagggaactgagtactcagtccatgattttgtctatgtgaaccctcatcaatttgccgtggaaagtggggagactgaaacttttaagggtggaaggaatgttggattgaagccttatgctgtgtgccagttattggaagtagttccaatggaaacaaaacaatctgaaacaagatccactgaggtcaaagttcaaagatttttcagaccagatgatatttcacctgaaaaggcatattgttctgatattcgtgagatatattatagtgaagaaacacatcttttatctgttgaggtgattgaagggaaatgtgaagtcagaaagaaaattgatattcccacatgcagtgctcctgcaatctttgatcataccttcttctgcgagcacatgtatgatccttccaatgggtccctcaagcagctaccagctcatatcaaatcaaagttttcagctgtgagtaaagatggtgatgttgcaagtagaaagagaaagggaaagtctaaagaaggagaaaatgatacagaagatgacaaacaattggaagcatctccagagtaccgtttggccactttggatatttttgctggttgtggtggcttgtctgagggattgcagcaagctggtgtctcaacaaccaagtgggctattgaatatgaagagcctgctggggaagcatttaaactcaatcatgcagaatcattgatgtttattaataattgcaatgtgatcctcagggctgtgatggagaggtgtggagatgcagatgactgtatttctacttctgaggctgctaaaatggcttcatcacttgatgcaaaggtgatcgatggtttgccattgccagggcaggtggatttcatcaatggagggcctccatgtcagggcttctctggaatgaataggtttaatcagagcacttggagtaaagttcaatgtgagatgatcttagcattcttatcctttgctgattacttccgcccaaagtatttcctcttagagaatgtgaggaactttgtatctttcaataaaggacagactttccgtttaactattgcttctcttcttcagatgggttatcaggtgagatttggaattttggaggccggagcatatggagtttctcagtcacgcaaacgtgcctttatatgggcagcctcccctgaggagatactcccagaatggccagagccaatgcatgtttttgctgccccagagttaaaaataacattgtctgagaagtcacaatatgctgctgttaggagtactgcttatggagcccctttccgtgcaataactgtcagagatacaattggtgatctcccagatgtggcgaatggtgcttctaagacaaatttggagtacggaaatgatcctatctcttggtttcaaaagaaaattcgaggcgacatggttgtcttgactgaccatatatcaaaagaaatgaatgagctgaatcttattagatgcaagaatatcccaaagcggccaggtgctgattggcgtgatctcccagatgaaaaggtcaaattgtctactgggcaaatggttgatttgataccatggtgccttccaaacacagctaaacgacacaatcagtggaaggggctatttggaagattggattgggaagggaacttcccaacatcaataacagatccccagccaatgggtaaggtggggatgtgctttcaccctgagcaggacaggattcttactgttcgtgaatgtgcacgatctcaaggattcccagataactatcagtttttcggtaacattcaacacaagcatcggcaaattggaaatgctgttcctcctcctctagcatatgcgttgggaaggaaacttaaggaagcactggatagtaagaggcagaaatag

SEQ ID NO: 129 - protein, 1549 aa, Populus balsamifera subsp. trichocarpa
  misc_feature (1)..(1549): Ceres GDNA ANNOT ID no. 1478748
MRKNKKGKQKSSVSNVKKEVPEKNTKGKKRNSPDTNKEEPTGGDGSLKRPKRAAACKDFKEKSVRLHEEKSYVVESKKEQVVDEEILAVRLTQGQEEGRPNRRLIDFVVHDANGNPQPLEMVEVDDMFISGIIMPHEESLDKEKEVHVRCDGFGRIEAWDISGYEDGSPVIWLSTEVADYDCIKPAGGYKKFFDHFFQKALACVEVYKKLSRFSGGNPEFTLDELLAGVVRAMSGNKCFSGAVSIKNFLISQGEFIYHQIIGLDETSTKNDKKFADLPVLVALRDESRNHGNVLIAKAANSGGNLVIGPESVDGAVVNQSNQSSTTVEEDEDAKLARLLQDEEYWQSNMRQKKSRGSVSASNTIYIKINEDEIANDYPLPAFYKHSNEETDEYIAVASDDVIDHPDDLPRRMLHNWSLYNSDSRLISLELLPMKPCEDIDVTIFGSGSMTEDDGSGFCLDDGPDQSSSRGLEAQDDMGLPIFLSAIKEWMIEFGSSMIFISLRTDMAWYRLGKPSKQYASWYKPVLKTVKLARTIITLLKEQSRVSRLSFADVIRKVSEFKKDHHAYISSDLAAVERYVVVHGQIILQLFAEFPDQKIKKCAFVVGLTRMMEERHHTKWVVNKKAIVQKCHSNLNPRAAMDTVASGASKRKLMQATTTRLINRIWGEYYSNYSPEDLKEGNDCDVKEEDELEEQDENEDDDKEVVVEKTLKPYSVFEHCKSHTSQKEVRWDGNPVRKISSGEDIYKQAIVCGQVIVVGAAVLVEVDEPDELPAIYFVEYMFETRNGSKMFHGRMMKWGSETVLGNTANDREVFLTNECMNYKLQDVKQTIILEVRKRPWGHHHRKDNANADRIDREKAEERKKKGLPLEYYCKSLYWPERGAFFTLPFDTMGLGSGVCHSCNLKISEEDKNISKVNSSQTGFSYKGTEYSVHDFVYVNPHQFAVESGETETFKGGRNVGLKPYAVCQLLEVVPMETKQSETRSTEVKVQRFFRPDDISPEKAYCSDIREIYYSEETHLLSVEVIEGKCEVRKKIDIPTCSAPAIFDHTFFCEHMYDPSNGSLKQLPAHIKSKFSAVSKDGDVASRKRKGKSKEGENDTEDDKQLEASPEYRLATLDIFAGCGGLSEGLQQAGVSTTKWAIEYEEPAGEAFKLNHAESLMFINNCNVILRAVMERCGDADDCISTSEAAKMASSLDAKVIDGLPLPGQVDFINGGPPCQGFSGMNRFNQSTWSKVQCEMILAFLSFADYFRPKYFLLENVRNFVSFNKGQTFRLTIASLLQMGYQVRFGILEAGAYGVSQSRKRAFIWAASPEEILPEWPEPMHVFAAPELKITLSEKSQYAAVRSTAYGAPFRAITVRDTIGDLPDVANGASKTNLEYGNDPISWFQKKIRGDMVVLTDHISKEMNELNLIRCKNIPKRPGADWRDLPDEKVKLSTGQMVDLIPWCLPNTAKRHNQWKGLFGRLDWEGNFPTSITDPQPMGKVGMCFHPEQDRILTVRECARSQGFPDNYQFFGNIQHKHRQIGNAVPPPLAYALGRKLKEALDSKRQK

SEQ ID NO: 130 - DNA, 4671 bp, Nicotiana tabacum
  misc_feature (1)..(4671): Public GI no. 56130955
atggcttattcttttttccatttttttgctggttattcaggacacaaaaaggagaaaagcaaacgagattctgtgtcaaaaaggaaggcacctgcaactgacaagaaggaaaagaaacagcctgtttctgaagctattgaggagcccactgctgcacgcaaaaggcccaagcgagctgctgcttgttcaaattttaaagagaaaaatgttcatttatcaaaaaattctgcagtcattgaaacaaagaaggaccaatgcgtagaggaagaggttttggctattcggttaactgcgggtctacaggattctcagcgaccctgtagaagactaacagattttatctttcataatttggaaggaataccacaaccttttgaaatgtctgaagttgatgatctgtttattactggtctcattttaccacttgaggacaataatgacaaagaaaaagcaaaaggaattagatgtgaaggctttgggcgtatagaagaatgggctatctctggctatgaagatggaactcctatcatatggatctcaacagagacagctgattatgattgtaaaaaaccctcaggtggctataagaagttttatgaccacttcttcgccaaagctacagcctgcattgaggtttacaaaaagctgtcgaaatcttctggaggaaatcctgatttaagccttgatgggttgcttgcaggggttgtccgagcaatgagtggtttaaaatgcttttcgggtggtgtatcaatcagggactttctcatttctcagggagagtttgtctataagcaacttatcggtcaggacgatacatcaaagaagactgatcagctttttgttgagttacctgtcctggcttcccttagagatgaaagcagcaatcaggaaatgctttcacaaccagagcctttatcatttggtaggactctaactataggtccaaaagtaggcaaaggagaaggcaagagagatcaatctgatttaaccactggtccagaacaagaagaggaagatctgaaattggccaaactgttacatgaacaggagtactggcactccttgaaccagaagacaagccgtagtacatcttcctcatctagcaaattttacatcaagatcaatgaggatgagattgcaagtgattatcctttacctgcatattacaagacatgtaatgaagagaccgatgagtatatcgtctttgacagtggggttgatacatactatattgatgacttgcctcgcagtatgcttcataattgggcattgtacaactcagactcaagactaatttcttcagagctcctgcctatgaaaccatgcgctgatattgatgtaaccatatttgggtctggagtgatgactgctgatgatggatctggatacaatgttgatgctgatgctaataactcctcttcaggtggttctggatcagctgagattgatggaatgccaatttatttgagtgcaataaaagaatggatgattgagtttgggtcctcgatgatctttatatctattcggactgatatggcctggtataggcttgggaagccatcaaaacagtatgctccttggtatgaaccagtcctaaagactgcgaagttggcagtgagcattattactttgttaaaggaacaaagtcgttgtgctagactttcttttggagatgtcattaaaagggtttcagagttcaagaaacaccatcctgcttatatatcatctaatacagatgtggtggaaagatatgtggttgtacatggacagattattctgcagcagttttcagaatttcctgatgaaagcattaggaaatgtgcatttgtgattggcctctcaaggaaaatggaggagaggcaccatacaaaatggttgattaagaagaagaaggttgtgcagagacatgaacagaacttaaatcctagagcatctatggcgccatctgtaaaaaggaaagctatgcaggctactacaacaagactaatcaacagaatctggggggagtactattccaattactcacctgagacgtcaaaggaggttgttgcttgtgaggtgaaggatgatgaagaagtagatgagcaggaggaaaatgacgaggatgatgctcaagaggagaacttggaagtttcagagaaaactcatacaccttgctctacaagaaggcatattaagtcacgttctgacagcaaagaaataaactgggatggggaatccataggtaaaacagcgtctggtgaactgttgtttaaaaagcctagaattcatggaaatgagattgctgttggagattcagttctggtggaacatgatgaaccagatgaacttccttctatttactttgtcgaatacatgtttgaaaaattggatggtagcaaaatgctccatggaagaatgatgcaacggggatctgaaactgtacttggaaatgcagctaatgaaagagaggtatttttgatcaatgaatgcatggatttgcaactaggagatgtcaaagaaagtgtagttgtcagtatcaggatgatgccatggggacatcagcatagaaaagcgaatgcttatgttgataaacttgatagagcaaaggcagaagacaggaagaagaagggattgccatccgaattttattgcaaaagcttttatcagcctgacagaggtgctttcttcagacttccgtttgataagatgggtcttggtaatggcttatgttactcctgtgagttgcagcaaattgatcaggaaaaggaatcttttaagttggatatgtccaactccagttttgtatatctggggactgagtattcaattgatgactttgtttatatacaccctgatcactttgctgtagaaagagggggaagtggaactttcaaagctgggagaaatgtggggttgatggcctatgtagtgtgtcaactaatagagatttctggccccaagggatctaaacaagctaaagtagattctaccaacgtcaaagtcaggagattcttcagaccagaggacatttcttcagataaggcatactcttctgatattcgggagatctactatagtgaggagatacatacagttccggtagaaacaattgaaggtaaatgtgaagtgaggaagaagtatgatattccgtctgaagatgtccctgccacctttgaccatgttttcttttgtgaatatttgtatgatccattgaatggatccctcaaacagttaccagctcaggtaaagctgagattctcaagagttaaactagatgatgctgcatctaggaagagaaagggaaaaggcaaggaaggagaggatgaactgagagttgggcaactaaatgtagcttctcaacagaatcgtttggccacactagatatctttgctggttgtggtggcctgtctgaggggttgcagcgttcgggtgtctcagatacaaaatgggcaattgaatatgaagagcctgctggagatgcgtttaaacttaatcatccagaggcaaaggtgttcatacagaattgcaatgtgattctgagggctgtcatgcaaaagtgtggagatgctgagaactgtatctcaacctcagaggcttctgaattagctgcagcaatggatgagaacgaactgaatagtttgccactgccaggacaagtggacttcataaatggaggccctccttgtcaggggttttctggaatgaatagatttaatcagagcacctggagtaaagttcagtgcgagatgattctggcatttttatcctttgctgattattatcggcctaagttctttctcttggagaatgttaggaattttgtgtcgttcaaccaaaaacaaacatttcgcttaactgttgcttcccttcttgagatgggttatcaggtgaggtttggtatccttgaagctggagcgtttggagttcctcagtctaggaagagagcatttatctgggctgcttccccagaggagattcttccagagtggccagaaccaatgcatgtatttggtgtcccagaattaaaaatcacattatctgaaacttgtcactatgcagctgtgaggagtactgctagtggagctccattccgttcgcttactgtcagagacacaattggagatcttcctgctgttggcaacggagcatccaagacctgtatagagtatcaagttgacccgatatcctggttccaaaggaaaattcggggcaactcaataacattatccgatcacattacgaaagagatgaacgagcttaacctaatcaggtgccaaagaattcctaagcggccaggagccgactggcgtgaccttccggatgaaaaggttaaactatgtaatggtcaactggttgatttgattccgtggtgcctgcctaacactgctaagaggcacaaccagtggaaggggctctttgggaggttggattgggatgggaacttccccacttcctttactgacccccagccgatgggtaaggtggggatgtgttttcatcccgaccaagacaggattgttacagttcgtgaatgtgcgcgttctcaaggtttcccagatagctatcaatttgctggtaacattttgcacaagcacaggcaaataggaaatgctgttccacctcctttggcatatgcactgggaaggaaacttaaggaagctgttgagagcaagaagaggtccacttag

SEQ ID NO: 131 - protein, 1556 aa, Nicotiana tabacum
  misc_feature (1)..(1556): Public GI no. 56130955
MAYSFFHFFAGYSGHKKEKSKRDSVSKRKAPATDKKEKKQPVSEAIEEPTAARKRPKRAAACSNFKEKNVHLSKNSAVIETKKDQCVEEEVLAIRLTAGLQDSQRPCRRLTDFIFHNLEGIPQPFEMSEVDDLFITGLILPLEDNNDKEKAKGIRCEGFGRIEEWAISGYEDGTPIIWISTETADYDCKKPSGGYKKFYDHFFAKATACIEVYKKLSKSSGGNPDLSLDGLLAGVVRAMSGLKCFSGGVSIRDFLISQGEFVYKQLIGQDDTSKKTDQLFVELPVLASLRDESSNQEMLSQPEPLSFGRTLTIGPKVGKGEGKRDQSDLTTGPEQEEEDLKLAKLLHEQEYWHSLNQKTSRSTSSSSSKFYIKINEDEIASDYPLPAYYKTCNEETDEYIVFDSGVDTYYIDDLPRSMLHNWALYNSDSRLISSELLPMKPCADIDVTIFGSGVMTADDGSGYNVDADANNSSSGGSGSAEIDGMPIYLSAIKEWMIEFGSSMIFISIRTDMAWYRLGKPSKQYAPWYEPVLKTAKLAVSIITLLKEQSRCARLSFGDVIKRVSEFKKHHPAYISSNTDVVERYVVVHGQIILQQFSEFPDESIRKCAFVIGLSRKMEERHHTKWLIKKKKVVQRHEQNLNPRASMAPSVKRKAMQATTTRLINRIWGEYYSNYSPETSKEVVACEVKDDEEVDEQEENDEDDAQEENLEVSEKTHTPCSTRRHIKSRSDSKEINWDGESIGKTASGELLFKKPRIHGNEIAVGDSVLVEHDEPDELPSIYFVEYMFEKLDGSKMLHGRMMQRGSETVLGNAANEREVFLINECMDLQLGDVKESVVVSIRMMPWGHQHRKANAYVDKLDRAKAEDRKKKGLPSEFYCKSFYQPDRGAFFRLPFDKMGLGNGLCYSCELQQIDQEKESFKLDMSNSSFVYLGTEYSIDDFVYIHPDHFAVERGGSGTFKAGRNVGLMAYVVCQLIEISGPKGSKQAKVDSTNVKVRRFFRPEDISSDKAYSSDIREIYYSEEIHTVPVETIEGKCEVRKKYDIPSEDVPATFDHVFFCEYLYDPLNGSLKQLPAQVKLRFSRVKLDDAASRKRKGKGKEGEDELRVGQLNVASQQNRLATLDIFAGCGGLSEGLQRSGVSDTKWAIEYEEPAGDAFKLNHPEAKVFIQNCNVILRAVMQKCGDAENCISTSEASELAAAMDENELNSLPLPGQVDFINGGPPCQGFSGMNRFNQSTWSKVQCEMILAFLSFADYYRPKFFLLENVRNFVSFNQKQTFRLTVASLLEMGYQVRFGILEAGAFGVPQSRKRAFIWAASPEEILPEWPEPMHVFGVPELKITLSETCHYAAVRSTASGAPFRSLTVRDTIGDLPAVGNGASKTCIEYQVDPISWFQRKIRGNSITLSDHITKEMNELNLIRCQRIPKRPGADWRDLPDEKVKLCNGQLVDLIPWCLPNTAKRHNQWKGLFGRLDWDGNFPTSFTDPQPMGKVGMCFHPDQDRIVTVRECARSQGFPDSYQFAGNILHKHRQIGNAVPPPLAYALGRKLKEAVESKKRST

SEQ ID NO: 132 - DNA, 4578 bp, Zea mays
  misc_feature (1)..(4578): Public GI no. 3132824
atgcagagcaaagccacaaaagaaggaagaggaatccacagaaaacaacaagctggagaatggatctctggatacaacagaagaggtgcatcatggagtcgaaaaagtgatggacatgttacccgcaagagaccaaggagatcagcggcctgttctgatttcaaagagaaatccatacgcttatccgaaaaaaaatctgttgtcatggtcaagaagaatcggatggaggaggaagaagtagatgctgtcaatctgacaaaacttggaccagaagatccaccaccttgccggaagttgatcgattttatcttgcatgatgcagaagggaacccacaaccctttgaaatgtcagaaattgatgacttctttataacagctcttatcatgcccatggatgatgatctagaaaaagagcgtgaaagaggagtacgctgtgaaggatttgggcgaattgaggactggaatatttctggttatgatgaaggtactcctgtaatctgggtgtcaactgatgttgctgactatgaatgtgtgaaaccatcaaccaattacaaatcttattttgaccacttctatgagaaggctcaggtgtgtgttgaagttttcaaaaagcttgcaaaatcagttggtgggaatcctaaccagggcctggatgaattgcttgctagtgttgttcggtcaaccaatgccatgaaaggatatagtggaaccatgagcaaagatttggtgatatccattggagaatttgtatacaatcaacttgttggtttggatgagacatcaaacaatgatgatgaaaagtttgctaccctgccagttcttctttctctaagagaccagtgcagatctagggtggaactgaccaagttgccctctaacttctcgaacacaagtctgaaaattaaggactcagagtgtgatgagacagcagaagacgatgatgatgcaaaattagctagattacttcaacaagaagaagaatggaaaatgatgaagaaacagaggggtaggcgtggaacaccatcccagaaaaatgtctacataaaaatcagtgaagctgagattgccaatgactatccccttcctgcatactataagccatttagccaggaaatggatgaatacatatttgatagtgatgacagcatattttctgatgatgtgccagttaggatactcaataactggacactgtacaatgcagattccaggcttatatctttggaattgatccctatgaaatcaggggcagaaaatgatgtggttgtctttggatctggtttcatgagagatgatgatggcagttgctgttctacagctgagtctgtgaaatcttcgtcttcctccagcaaagctgaccaactggatgcgggaatccctatttatttgagcccaatcaaagaatggattatagagtttggtggctcaatgatttgtataaccattcggactgatgtggcctggtacaagctacgccaaccaacaaaacaatatgctccatggtgtgagcctgtactgaaaacagcaaggcttgctgttagcatcattaccctgttgaaagagcagagtcgtgcctcaaagctttcttttgctgatgtcataagaaaagtagctgaatttgacaaaggaaaccctgcatttatatcttcaaacatcacacttgttgagagatatattgtggtgcatggacagataatactccagcagtttgcagattttccagatgagactattcgtcggagtgcatttgtcagtggtcttttattgaagatggaacagaggaggcatacaaagttagttatgaagaaaaaaactcaagtgatgaggggagagaatctgaatccaagtgcagcaatgggtccagcatcgaggaaaaaagcaatgcgtgcaacaacaaccaggctcatcaacagaatctggagtgattactatgcacatcatttccctgaagattccaaggagggagatggaaatgaaacaaaagaaattgatgatgaacaagaagaaaatgaagatgaggatgctgaagatgaaggacagattgaggagaacatctcaaagactcctccatcaacacggtcccggaagttgctatcacaaacttgtaaggaaatcagatgggaaggtgaaacatctgggaaaacattgtctggagaaactctatataaatgtgcttatgttagggaactcagaatacctgttggtggaacagtggctctagaagatgattcaggagacacagtcatttgttttgttgagtacatgttccagaaagttgatggttcaaaaatggttcatgggaggattctgcaaaaagggtcacagacaattcttggcaatgcagcaaatgagagggaggttttcttaactaatgactgcttagaattcaaattagatgacatcaaggaattggtaatggttgatatccaatcaaggccttggggtcacaagtacagaaaagagaattctgaagctgataaagttgagcaggtcaaagcagaagagagaaagaaaaagggccagcccatggtatatttctgcaaaagcttgtactggcctgagaagggtgccttctttgccctctcccgagataaaatgggtcttggtagtggtttatgtagttcttgtgataatatagagccagattctgatgaattgaaaatattctcgaagaccagctttgtctacagaaaggttacatataatgtcaatgagtttttatacataagacctgatttttttgctgaagatgaggatcgtgcaaccttcaaggctggccgaaatgtgggtctaaagccctatgcagtttgtcaaatattgtccatccctgaaggggctggatctaaaaaactcaatccagcatcagcaaatatcagtgctagaagattttacagaccagatgacatttcatcagccaaagcctatgcatctgacatcagagaggtctactatagtgaggatgtaattgatgtgcctgtggatatgatagagggaaaatgtgaggttagaaagaagaacgatcttgcaagttcagaccttccagtgatgtttgaacatgtatttttctgtgaacttatatatgaccgtgccagtggagctctcaagcagttgcctccaaatgttaggtttatgtctatggtgcaaaggacaagtgcgttgaaaaagaacaaaggaaagcagatctgtgagcctgatcaaatagattcaggtaaatggttggatgtgcctaaagagaaccgtctagctactcttgacatttttgctggctgtggaggtttatcagaagggctgcagcaagctggtgtatcttttacaaaatgggcgattgaatacgaagagcctgctggtgaagcatttaataaaaatcatccagaggctgtggtctttgtagataactgcaatgtgattctaaaggcaattatggataaatgtggggatactgatgattgtgtttcaacttctgaagctgctgaacaagcagcaaaacttccagaagtgaacattaataatcttccagtccctggcgaagttgaattcataaatggtggtcctccgtgtcagggattctctgggatgaatagattcaaccaaagcccatggagtaaagttcagtgtgagatgattctagcattcctctcattcgctgagtatttccgtcccagattctttctgttagaaaatgttcggaactttgtttccttcaacaaagggcagaccttccgtttggcagttgcatctcttctggagatgggataccaggtccggtttggaattctagaagcaggggcttttggtgttgcccagtccaggaaaagggcgtttatttgggctgctgcacctggagagatgcttcctgattggccagagccgatgcatgtgtttgctagccctgagctgaagataacactgcctgatggccaatactatgcagctgcaagaagcactgctggtggagcgcctttccgagcgattactgttagagatacaattggggatctgcctaaagtgggaaatggtgccagcaaactcacgcttgagtatggaggtgagcccgtgtcttggttccagaagaagataagagggagtatgatggtactgaatgatcacatatctaaggagatgaatgagctgaacctaataaggtgtcaacacattccgaaacggccgggttgtgattggcatgacctaccggacgagaaggttaagctgtcaaatgggcagatggctgacctgataccttggtgcctgcccaacacagccaagaggcacaatcagtggaaaggactgtacgggaggctggactgggaaggcaacttccccacatccgtcactgatccccagccaatgggcaaggtcggcatgtgcttccaccctgatcaagacaggatcatcacagtccgggaatgtgctcggtcacagggctttcctgacagctatgaatttgcgggcaacatccagaacaagcaccggcagattggcaatgccgtgcccccgcctcttgcctatgcacttgggaggaagctcaaggaagccgttgacaagcgtcaggaagccagcgcaggcgtgcctgcaccatga

SEQ ID NO: 133 - protein, 1525 aa, Zea mays
  misc_feature (1)..(1525): Public GI no. 3132825
MQSKATKEGRGIHRKQQAGEWISGYNRRGASWSRKSDGHVTRKRPRRSAACSDFKEKSIRLSEKKSVVMVKKNRMEEEEVDAVNLTKLGPEDPPPCRKLIDFILHDAEGNPQPFEMSEIDDFFITALIMPMDDDLEKERERGVRCEGFGRIEDWNISGYDEGTPVIWVSTDVADYECVKPSTNYKSYFDHFYEKAQVCVEVFKKLAKSVGGNPNQGLDELLASVVRSTNAMKGYSGTMSKDLVISIGEFVYNQLVGLDETSNNDDEKFATLPVLLSLRDQCRSRVELTKLPSNFSNTSLKIKDSECDETAEDDDDAKLARLLQQEEEWKMMKKQRGRRGTPSQKNVYIKISEAEIANDYPLPAYYKPFSQEMDEYIFDSDDSIFSDDVPVRILNNWTLYNADSRLISLELIPMKSGAENDVVVFGSGFMRDDDGSCCSTAESVKSSSSSSKADQLDAGIPIYLSPIKEWIIEFGGSMICITIRTDVAWYKLRQPTKQYAPWCEPVLKTARLAVSIITLLKEQSRASKLSFADVIRKVAEFDKGNPAFISSNITLVERYIVVHGQIILQQFADFPDETIRRSAFVSGLLLKMEQRRHTKLVMKKKTQVMRGENLNPSAAMGPASRKKAMRATTTRLINRIWSDYYAHHFPEDSKEGDGNETKEIDDEQEENEDEDAEDEGQIEENISKTPPSTRSRKLLSQTCKEIRWEGETSGKTLSGETLYKCAYVRELRIPVGGTVALEDDSGDTVICFVEYMFQKVDGSKMVHGRILQKGSQTILGNAANEREVFLTNDCLEFKLDDIKELVMVDIQSRPWGHKYRKENSEADKVEQVKAEERKKKGQPMVYFCKSLYWPEKGAFFALSRDKMGLGSGLCSSCDNIEPDSDELKIFSKTSFVYRKVTYNVNEFLYIRPDFFAEDEDRATFKAGRNVGLKPYAVCQILSIPEGAGSKKLNPASANISARRFYRPDDISSAKAYASDIREVYYSEDVIDVPVDMIEGKCEVRKKNDLASSDLPVMFEHVFFCELIYDRASGALKQLPPNVRFMSMVQRTSALKKNKGKQICEPDQIDSGKWLDVPKENRLATLDIFAGCGGLSEGLQQAGVSFTKWAIEYEEPAGEAFNKNHPEAVVFVDNCNVILKAIMDKCGDTDDCVSTSEAAEQAAKLPEVNINNLPVPGEVEFINGGPPCQGFSGMNRFNQSPWSKVQCEMILAFLSFAEYFRPRFFLLENVRNFVSFNKGQTFRLAVASLLEMGYQVRFGILEAGAFGVAQSRKRAFIWAAAPGEMLPDWPEPMHVFASPELKITLPDGQYYAAARSTAGGAPFRAITVRDTIGDLPKVGNGASKLTLEYGGEPVSWFQKKIRGSMMVLNDHISKEMNELNLIRCQHIPKRPGCDWHDLPDEKVKLSNGQMADLIPWCLPNTAKRHNQWKGLYGRLDWEGNFPTSVTDPQPMGKVGMCFHPDQDRIITVRECARSQGFPDSYEFAGNIQNKHRQIGNAVPPPLAYALGRKLKEAVDKRQEASAGVPAP

SEQ ID NO: 134 - DNA, 5681 bp, Oryza sativa
  misc_feature (1)..(5681): Ceres CDNA ID no. 23965502
atggtgaggcgggcgagcgtggcggcgtgcctgacctgcccgctctgcggccgcctcctccgcgacgccaccaccatctccgagtgcctccacacctcttatggtattttcaattgtgttttgttcagagttgatcacagtatgcaattcgtaaggtcaaggatatttcctttcaaacgaaggaaggttgagaacccagaaatcatttgtccagttgcgtcacctgtcaagaggaaagaaagatcactatcttcacttacaatccctgctcctcaggtgtctatacagaaatgtttgacaaagaggagaacgaaagcttcgtgcttacgcaactttcctttgcattctacctcgaggggcagtaaagatacatcaaagaaacttgggggttggagaccattgggttgtcaacttaagcttggcaaagacaaaaaatctctcaaatcaagtgtaaaagatacaaacagaaccaaaagtaagtctggtgatacagatgatggtgctcctgctagtaaagcaaaggctagagaaccctttacaagatatgggcgtgcagctaagaggactggaagaaagaaattgctcatgttgaagaacaaaaagaaaaggttcaaggcaaagcagcccagtaaaaagaggagattccgagcactatggttttatctacttgctgcttttgaccagagaggagtaccaactttaccacaactaccagcaaagtatttgaggatcaaggatgttgatttgcctgcttctattatacagaagtaccttgcacagaaacttaacctctcaagtgaaactgaggtagaagtgttgtgtggtggcaaagtagtgaaccaagggatgacactgcatgatctagcagattgctggcttgagaaaggaccaaagagccgaatgcgctcatcggggttgcgaactcggcgatggatcagtgacccgccgtggggagctatggcgaaaagtccacgttctgttgttaccacaggaacaaaaaggcgtagagcaaaggttcataaagaagatgagcctgttgagaatgaaaacttggagagtgaatttgatgtttccaagaaagagagcaatggtgccactgaacctggtaatgagcctgttgccagcaagagaccgaagagagcagctgcctgttctaacttcaaagagaagtcattggacttatcagaaaaagattcaattatcacaatcaaggaaagtcgggttgaagagaaggaaatagaggctgttaatttgacaaggacgggacctgaagatggtcaaccttgcagaaaaatcatcgatttcatcttacatgatggagatggtaatctgcaaccctttgaaatgtctgaagttgatgacattttcataacagctcttatcatgcccttggatgatgatctggaaaaggataggggaaagggaatatgttgttcggggtttggacgaattgaaaactgggcgatttctggctatgatgaaggtgctgcagtaatttgggtctcaacagaaacatcagattacaaatgtgtgaagccagcaagcagttacagatcttattttgaacactttagtgagaaggcacgtgtctgtgttgaagtctataagaagttagctagatcagttggtggaaatcctcaggtggacttagaagaattaattgctggtgttgtccgttccattaattcaaacagaagcttcaacggaacagtaaccaaagactttgtgatctcctctggtgagttcatatataaacagcttattggattagaccatacagctggcaatgatgatgagatgttggccacactgccagttcttgttgcactgaaagatgaatgtaaatcaagagcaggattcacacatttgccagctatgccctcgaatggaactctgaggattaaggatgggcaagacaagggactgactgaggatgaggatgcaaaattagcaagactgttgcaggaagaggaagaatggaaaatgatgaagcagagaggcaagcgtggaacttcacagaaaaatatctacatcaagatttgtgaaactgaaattgccaacgactacccacttccagcctactataaaccatataaccaagaaatggatgagtacatatttgatagtgatattggtatgtattctgatgatgtacctgtaagaatccttgacaactgggctctatacaattcagattccagactcatttctttggagctcatccctatgaaagctggtgcagaaaatgatattgtggtatttggatctggttttatgagagaggatgatggtagttgctgttcaacagctgagctagcacagttacattcttcctcaagtaaatctggccgggaagatccaggagttccaatttatttgagcccaattaaagagtgggttgtagaatttggtggttcaatgatctgcataaccattcgaactgacgttgcttggtacaaattacgccagccaacaaagcaatatgctccatggtgtgagcctgtgctgaaaacagcaaggctagctgttagtatcatcacccttttaaaagagcaaagtcgcgcttcaaagctttcttttgctgaagttatcaagaaagtagcagaatttgacagtagacaccctgcatttatatcatcgaaagcaccaaccgttgaaagatatgtcgtggtgcatggacagataatacttcagcagtttgcagactttccagatgaatctgtcaaacggtgtgccttcatcacaggtcttctagcaaagatggaggaaagtaggcacacaaagttggccatcaagaaaaaatctcaacagatgagaggggagaatctgaacccaagcgcaaaaatgggtccaatactgagaaagaagcttatgcgtgctacaactacaatgttgatcagcaagatatggggtgaatactatgccacttatttccctggggatacaaaggaagaagatcagaatgaaccaaaggaaattgatgatgatcaagaagaaaatgaagacaatgatgctgaagaggaggtaaatgttcaagatgagaaggccacaaggactccaccatcaacacggtctagaaagtcgtcagcagatactcgcaaggaaatcaaatgggaaggtcaaacagctggaaaaacagtgtctggagaagttctgtacaaatgtgttattgttcaagacctcagtatttctgttggtgcgacagtcacaacagaggatgattcaggagaaaccatcatgtgttttgttgagtatatgtatgagaaacttgatggtaaaaatatgattcatgggataattctgcaagaaggttcacagactgttcttggcaatgctgcaaatgatagagaggttttcttgactaatgactgtttagaatttgaagcaagtgacatcaaagagttggtgactgttaatatccaatcactgccttggggccacaagtacagaaaagagaattctgaagctaagagaattgaaaaggccaaggcagaggagaggaaaaggaagggcctgccagtggaatatatttgcaaaagcttatactggcctgagaaaggtggattcttctcccttccgtatgataaaattggaaatggcacaggcatctgtagctcctgtgagagaaaaccagttggcaatgaattcaagttactttctgagagcagctttgtctttgagaatattacgtataacatccatgactttctgtatatcaggcctgaatttttctcccaaggggagggccatgagacctacaaggctggaaggaatgtgggtctaaaaccttatgcagtctgccatctgctgagtgttcatggtcctgctggatcaaggaaagctaatccagaatcgacaaaagtgaaagtaagaaggttttaccgacctgatgacatttcatcaacaaaagcctactcatcagacatccgagaggtgtactacagtgaagatataataagtgtacctgtggtgatgatagagggaaaatgtgaggttcgactgaaggatgaccttccaaattcagatcttccagcggtggttgaacatgtcttttgttgtgaatatttatatgatcctgctaatggagctctcaaacagctaccgcccaatgttagacttgtgacactgacaaggaaggtacctgcttcaaaaaagaacaaaggaaagcaaatttgtgacattgagctaggtggttcagacaaaccaaaggatgggcaatcagagaactgtcttgcaacacttgacatttttgctggttgtggaggtttatctgaaggattgcagcgatcaggattgtcacttactaaatgggctattgaatatgaagaacctgctggggatgcatttggtgaaaaccatccagaagctgcagtatttgtcgaaaactgcaatgtgattctgaaggcaattatggacaagtgtggtgattctgatgattgcatctccacttctgaggctgctgaacgagcagctaaactttctgaggacaagattaagaatctgcccgtgcctggcgaagtagaattcataaatggtggccctccgtgtcagggtttttctgggatgaacagattcaatcaaagtccctggagcaaagtccagtgcgagatgatcttagcattcctgtcatttgcggagtatttccgtcctagattctttctcttagaaaatgttaggaactttgtctcgttcaacaaaggacagaccttcagattgacactggcatcactcctggagatgggataccaggtccgatttggaattttagaggcaggggcttatggtgttgcgcagtccaggaaaagggcattcatttgggccgctgcacctggagagactcttccagagtggcctgaaccaatgcacgtctttgctagccctgagctgaaaataactctacctgatggcaagttctacgccgctgtcaagagcaccgctgcaggagcccctttccgctcaattacagttcgagatacaattggggatctaccagctgtggaaaatggcgccggcaaaccaacaattcagtacggaagcggtcctgtgtcttggttccagaagaagattagaagcgacatggcttcactgaatgaccacatatctaaagagatgaatgagctgaacctcataagatgcaagcacattccaaagcgcccaggttgcgactggcatgacctgccagatgaaaaggtgaagctgtccacagggcagatggtggacttgatcccttggtgcttgcccaacacagccaaaaggcacaatcagtggaaaggactgtacggtaggttggactgggagggcaatttccccacttctgtaacggatcctcagccaatggggaaggtcggcatgtgcttccatcctgagcaggacaggatcattactgtccgtgaatgtgctcgatcccagggcttccccgatagctaccgtttcgctggcaacatccagaacaagcacaggcagatcgggaatgccgtgccaccgccccttgcctatgccctcgggaggaagctcaagcaagccatcgacgccaagcgttgagtggcttttaacttcactgcatcgccctcattttttggtcggtccaaataggtttaactaagcattacagttttctatattttgtgagcaattggactcctaaaattaattctgggatggttacatggattaccttttgt

SEQ ID NO: 135 - protein, 1846 aa, Oryza sativa
  misc_feature (1)..(1846): Ceres CDNA ID no. 23965502
MVRRASVAACLTCPLCGRLLRDATTISECLHTSYGIFNCVLFRVDHSMQFVRSRIFPFKRRKVENPEIICPVASPVKRKERSLSSLTIPAPQVSIQKCLTKRRTKASCLRNFPLHSTSRGSKDTSKKLGGWRPLGCQLKLGKDKKSLKSSVKDTNRTKSKSGDTDDGAPASKAKAREPFTRYGRAAKRTGRKKLLMLKNKKKRFKAKQPSKKRRFRALWFYLLAAFDQRGVPTLPQLPAKYLRIKDVDLPASIIQKYLAQKLNLSSETEVEVLCGGKVVNQGMTLHDLADCWLEKGPKSRMRSSGLRTRRWISDPPWGAMAKSPRSVVTTGTKRRRAKVHKEDEPVENENLESEFDVSKKESNGATEPGNEPVASKRPKRAAACSNFKEKSLDLSEKDSIITIKESRVEEKEIEAVNLTRTGPEDGQPCRKIIDFILHDGDGNLQPFEMSEVDDIFITALIMPLDDDLEKDRGKGICCSGFGRIENWAISGYDEGAAVIWVSTETSDYKCVKPASSYRSYFEHFSEKARVCVEVYKKLARSVGGNPQVDLEELIAGVVRSINSNRSFNGTVTKDFVISSGEFIYKQLIGLDHTAGNDDEMLATLPVLVALKDECKSRAGFTHLPAMPSNGTLRIKDGQDKGLTEDEDAKLARLLQEEEEWKMMKQRGKRGTSQKNIYIKICETEIANDYPLPAYYKPYNQEMDEYIFDSDIGMYSDDVPVRILDNWALYNSDSRLISLELIPMKAGAENDIVVFGSGFMREDDGSCCSTAELAQLHSSSSKSGREDPGVPIYLSPIKEWVVEFGGSMICITIRTDVAWYKLRQPTKQYAPWCEPVLKTARLAVSIITLLKEQSRASKLSFAEVIKKVAEFDSRHPAFISSKAPTVERYVVVHGQIILQQFADFPDESVKRCAFITGLLAKMEESRHTKLAIKKKSQQMRGENLNPSAKMGPILRKKLMRATTTMLISKIWGEYYATYFPGDTKEEDQNEPKEIDDDQEENEDNDAEEEVNVQDEKATRTPPSTRSRKSSADTRKEIKWEGQTAGKTVSGEVLYKCVIVQDLSISVGATVTTEDDSGETIMCFVEYMYEKLDGKNMIHGIILQEGSQTVLGNAANDREVFLTNDCLEFEASDIKELVTVNIQSLPWGHKYRKENSEAKRIEKAKAEERKRKGLPVEYICKSLYWPEKGGFFSLPYDKIGNGTGICSSCERKPVGNEFKLLSESSFVFENITYNIHDFLYIRPEFFSQGEGHETYKAGRNVGLKPYAVCHLLSVHGPAGSRKANPESTKVKVRRFYRPDDISSTKAYSSDIREVYYSEDIISVPVVMIEGKCEVRLKDDLPNSDLPAVVEHVFCCEYLYDPANGALKQLPPNVRLVTLTRKVPASKKNKGKQICDIELGGSDKPKDGQSENCLATLDIFAGCGGLSEGLQRSGLSLTKWAIEYEEPAGDAFGENHPEAAVFVENCNVILKAIMDKCGDSDDCISTSEAAERAAKLSEDKIKNLPVPGEVEFINGGPPCQGFSGMNRFNQSPWSKVQCEMILAFLSFAEYFRPRFFLLENVRNFVSFNKGQTFRLTLASLLEMGYQVRFGILEAGAYGVAQSRKRAFIWAAAPGETLPEWPEPMHVFASPELKITLPDGKFYAAVKSTAAGAPFRSITVRDTIGDLPAVENGAGKPTIQYGSGPVSWFQKKIRSDMASLNDHISKEMNELNLIRCKHIPKRPGCDWHDLPDEKVKLSTGQMVDLIPWCLPNTAKRHNQWKGLYGRLDWEGNFPTSVTDPQPMGKVGMCFHPEQDRIITVRECARSQGFPDSYRFAGNIQNKHRQIGNAVPPPLAYALGRKLKQAIDAKR

SEQ ID NO: 136 - DNA, 4374 bp, Zea mays
  misc_feature (1)..(4374): Public GI no. 20977598
atggtcaagaagaatcggatggaggaggaagaagtagatgctgtcaatcttacaaaacttggaccagaagatccaccaccttgccggaagttgatcgattttatcttgcatgatgcagaagggaacccacaaccctttgaaatgtcagaaattgatgacttctttataacagctcttatcatgcccatggatgatgatctagaaaaagagcgtgaaagaggagtacgctgtgaaggatttgggcgaattgaggactggaatatttctggttatgatgaaggtactcctgtaatctgggtgtcaactgatgttgctgactatgaatgtgtgaaaccatcaaccaattacaaatcttattttgaccacttctatgagaaggctcaggtgtgtgttgaagttttcaaaaagcttgcaaaatcagttggtgggaatcctaaccagggcctggatgaattgcttgctagtgttgttcggtcaaccaatgccatgaaaggatatagtggaaccatgagcaaagatttggtgatatccattggagaatttgtatacaatcaacttgttggtttggatgagacatcaaacaatgatgatgaaaagtttgctaccctgccagttcttctttctctaagagaccagtgcagatctagggtggaactgaccaagttgccctctaacttctcgaacacaagtctgaaaattaaggactcagagtgtgatgagacagcagaagacgatgatgatgcaaaattagctagattacttcaacaagaagaagaatggaaaatgatgaagaaacagaggggtaggcgtggaacaccatcccagaaaaatgtctacataaaaatcagtgaagctgagattgccaatgactatccccttcctgcatactataagccatttagccaggaaatggatgaatacatatttgatagtgatgacagcatattttctgatgatgtgccagttaggatactcaataactggacactgtacaatgcagattccaggcttatatctttggaattgatccctatgaaatcaggggcagaaaatgatgtggttgtctttggatctggtttcatgagagatgatgatggcagttgctgttctacagctgagtctgtgaaatcttcgtcttcctccagcaaagctgaccaactggatgcgggaatccctatttatctgagcccaatcaaagaatggattatagagtttggtggctcaatgatttgtataaccattcggactgatgtggcctggtacaagctacgccaaccaacaaaacaatatgctccatggtgtgagcctgtactgaaaacagcaaggcttgctgttagcatcattaccctgttgaaagagcagagtcgtgcctcaaagctttcttttgcagatgtcataagaaaagtagctgaatttgacaaaggaaaccctgcatttatatcttcaaacatcacacttgttgagagatatattgtggtgcatggacagataatactccagcagtttgcagattttccagatgagactatccgtcggagtgcatttgtcagtggtcttttattgaagatggaacagaggaggcatacaaagttagttatgaagaaaaaaactcaagtgatgaggggagagaatctgaatccaagtgcagcaatgggtccagcatcgaggaaaaaagcaatgcgtgcaacaacaaccaggctcatcaacagaatctggagtgattactatgcacatcatttccctgaagattccaaggagggagatggaaatgaaataaaagaaattgatgatgaacaagaagaaaatgaagatgaggatgctgaagatgaaggacagattgaagagaacatctcaaagactccttcatcaacacggtcccggaagttgctatcacaaacttgtaaggaaatcagatgggaaggtgaaacatctgggaaaacattgtctggagaaactctatataaatgtgcttatgttagggaactcagaatacctgttggtggaacagtagctctagaagatgattcaggagacacagtcatgtgttttgttgagtacatgttccagaaagttgatggttcaaaaatggttcatgggaggattctgcaaaaaggttcacagacaattcttgacaatgcggcaaatgagagggaggttttcttaactaatgactgcttagaattcaaattagatgacatcaaagaattggtaatggttgatatccaatcaaggccttggggtcacaagtacagaaaagagaattctgaagctgataaagttgagcaggtcaaagcagaagagagaaagaaaaagggccagcccatggtatatttctgcaaaagcttgtactggcctgagaagggtgccttctttgccctctcccgagataaaatgggtcttggtagtggtttatgtagttcttgtgataatatagagccagattctgatgaattaaaaatattctcgaagaccagctttgtctacagaaaggttacatataatgtcaatgagtttttatacataagccctgatttttttgctgaagatgaggatcgtgcaaccttcaaggctggccgaaatgtgggtctaaagccctatgcagtttgtcaaatattatccatccctgaaggggctggatctaaaaaactcaatccagcatcagcaaatatcagtgctagaagattttacagaccagatgacatttcatcagccaaagcctatgcttctgacatcagagaggtctactatagtgaggatgtaattgatgtgcctgtggatatgatagagggaaaatgtgaggttagaaagaagaacgatcttgcaagttcagaccttccagtgatgtttgaacatgtatttttctgtgaacttatatatgaccgtgccagtggagctctcaagcagttgcctccaaatgttaggtttatgtctatggtgcaaaggacaagtgcgttgaaaaagaacaaaggaaagcagatctgtgagcctgatcaaatagattcaggtaaatggttggatgtgcctaaagagaaccgtctagctactcttgacatttttgctggctgtggaggtttatcagaagggctgcagcaagctggtgtatcttttacaaaatgggcgattgaatacgaagagcctgctggtgaagcatttaataaaaatcatccagaggctgtggtctttgtagataactgcaatgtgattctaaaggcgattatggataaatgtggggatactgatgattgtgtttcaacttctgaagctgctgaacaagcagcaaaacttccagaagtgaacattaataatcttccagtccctggcgaagttgaattcataaatggtggtcctccgtgtcagggattctctgggatgaatagattcaaccaaagcccatggagtaaagttcagtgtgagatgattctagcattcctctcattcgctgagtatttccgtcccagattctttctgttagaaaatgttcggaactttgtttccttcaacaaagggcagaccttccgtttggcagttgcatctcttctggagatgggataccaggtccggtttggaattctagaagcaggggcttttggtgttgcccagtccaggaaaagggcgtttatttgggctgctgcacctggagagatgcttcctgattggccagagccgatgcatgtgtttgctagccctgagctgaagataacactgcctgatggccaatactatgcagctgcaagaagcactgctggtggagcgcctttccgagcgattactgttagagatacagttggggatctgcctaaagtgggaaatggtgccagcaaactcacgcttgagtatggaggtgagcctgtgtcttggttccagaagaagataagagggaatatgatggtactgaatgatcacatatctaaggagatgaatgagctgaacctaataaggtgtcaacacattcagaaacggccgggttgtgattggcatgacctaccggacgagaaggttaagctgtcaaatgggcagatggctgacctgataccttggtgcctgcccaacacagccaagaggcacaatcagtggaaaggactgtacgggaggctggactgggaaggcaacttccccacatccgtcactgatccccagccaatgggcaaggtcggcatgtgcttccaccctgatcaagacaggatcatcacagtccgggaatgtgctcggtcacagggctttcctgacagctatgaatttgcgggcaacatccagaacaagcaccggcagattggcaatgccgtgcccccgcctcttgcctatgcacttgggaggaagctgaaggaagccgttgacaagcgtcaggaagccagcgcaggcgtgcctgcaccatga

SEQ ID NO: 137 - protein, 1457 aa, Zea mays
  misc_feature (1)..(1457): Public GI no. 20977598
MVKKNRMEEEEVDAVNLTKLGPEDPPPCRKLIDFILHDAEGNPQPFEMSEIDDFFITALIMPMDDDLEKERERGVRCEGFGRIEDWNISGYDEGTPVIWVSTDVADYECVKPSTNYKSYFDHFYEKAQVCVEVFKKLAKSVGGNPNQGLDELLASVVRSTNAMKGYSGTMSKDLVISIGEFVYNQLVGLDETSNNDDEKFATLPVLLSLRDQCRSRVELTKLPSNFSNTSLKIKDSECDETAEDDDDAKLARLLQQEEEWKMMKKQRGRRGTPSQKNVYIKISEAEIANDYPLPAYYKPFSQEMDEYIFDSDDSIFSDDVPVRILNNWTLYNADSRLISLELIPMKSGAENDVVVFGSGFMRDDDGSCCSTAESVKSSSSSSKADQLDAGIPIYLSPIKEWIIEFGGSMICITIRTDVAWYKLRQPTKQYAPWCEPVLKTARLAVSIITLLKEQSRASKLSFADVIRKVAEFDKGNPAFISSNITLVERYIVVHGQIILQQFADFPDETIRRSAFVSGLLLKMEQRRHTKLVMKKKTQVMRGENLNPSAAMGPASRKKAMRATTTRLINRIWSDYYAHHFPEDSKEGDGNEIKEIDDEQEENEDEDAEDEGQIEENISKTPSSTRSRKLLSQTCKEIRWEGETSGKTLSGETLYKCAYVRELRIPVGGTVALEDDSGDTVMCFVEYMFQKVDGSKMVHGRILQKGSQTILDNAANEREVFLTNDCLEFKLDDIKELVMVDIQSRPWGHKYRKENSEADKVEQVKAEERKKKGQPMVYFCKSLYWPEKGAFFALSRDKMGLGSGLCSSCDNIEPDSDELKIFSKTSFVYRKVTYNVNEFLYISPDFFAEDEDRATFKAGRNVGLKPYAVCQILSIPEGAGSKKLNPASANISARRFYRPDDISSAKAYASDIREVYYSEDVIDVPVDMIEGKCEVRKKNDLASSDLPVMFEHVFFCELIYDRASGALKQLPPNVRFMSMVQRTSALKKNKGKQICEPDQIDSGKWLDVPKENRLATLDIFAGCGGLSEGLQQAGVSFTKWAIEYEEPAGEAFNKNHPEAVVFVDNCNVILKAIMDKCGDTDDCVSTSEAAEQAAKLPEVNINNLPVPGEVEFINGGPPCQGFSGMNRFNQSPWSKVQCEMILAFLSFAEYFRPRFFLLENVRNFVSFNKGQTFRLAVASLLEMGYQVRFGILEAGAFGVAQSRKRAFIWAAAPGEMLPDWPEPMHVFASPELKITLPDGQYYAAARSTAGGAPFRAITVRDTVGDLPKVGNGASKLTLEYGGEPVSWFQKKIRGNMMVLNDHISKEMNELNLIRCQHIQKRPGCDWHDLPDEKVKLSNGQMADLIPWCLPNTAKRHNQWKGLYGRLDWEGNFPTSVTDPQPMGKVGMCFHPDQDRIITVRECARSQGFPDSYEFAGNIQNKHRQIGNAVPPPLAYALGRKLKEAVDKRQEASAGVPAP

SEQ ID NO: 138 - DNA, 5286 bp, Daucus carota; identical to SEQ ID NO: 124
  misc_feature (1)..(5286): Public GI no. 2895089

SEQ ID NO: 139 - protein, 1761 aa, Daucus carota; identical to SEQ ID NO: 125
  misc_feature (1)..(1761): Public GI no. 2895089

SEQ ID NO: 140 - DNA, 4671 bp, Nicotiana tabacum; identical to SEQ ID NO: 130
  misc_feature (1)..(4671): Public GI no. 56130955

SEQ ID NO: 141 - protein, 1556 aa, Nicotiana tabacum; identical to SEQ ID NO: 131
  misc_feature (1)..(1556): Public GI no. 56130955

SEQ ID NO: 142 - DNA, 4680 bp, Lycopersicon esculentum; identical to SEQ ID NO: 126
  misc_feature (1)..(4680): Public GI no. 2887280

SEQ ID NO: 143 - protein, 1559 aa, Lycopersicon esculentum; identical to SEQ ID NO: 127
  misc_feature (1)..(1559): Public GI no. 2887280

SEQ ID NO: 144 - DNA, 4650 bp, Populus balsamifera subsp. trichocarpa; identical to SEQ ID NO: 128
  misc_feature (1)..(4650): Ceres GDNA ANNOT ID no. 1478748

SEQ ID NO: 145 - protein, 1549 aa, Populus balsamifera subsp. trichocarpa; identical to SEQ ID NO: 129
  misc_feature (1)..(1549): Ceres GDNA ANNOT ID no. 1478748

(19) United States
(12) Patent Application Publication
(10) Pub. No.: US 2006/0236421 A1
(43) Pub. Date: Oct. 19, 2006

(54) Secondary metabolite production via manipulation of genome methylation

(76) Inventors: Roger I. Pennell, Malibu, CA (US); David Vandinh Dang, Oak Park, CA (US); Nestor Apuya, Culver City, CA (US)

Correspondence Address: FISH & RICHARDSON P.C., PO BOX 1022, MINNEAPOLIS, MN, 55440-1022, US

(21) Appl. No.: 11/404,951

(22) Filed: Apr. 14, 2006

Related U.S. Application Data
(60) Provisional application No. 60/671,209, filed on Apr. 14, 2005.
(60) Provisional application No. 60/733,588, filed on Nov. 4, 2005.

(51) Int. Cl. A01H 1/00; C12N 5/04; C12N 15/82

(52) U.S. Cl. 800/278; 435/419; 435/468

(57) ABSTRACT

Methods for modulating the rate of production and accumulation of secondary metabolites, e.g., alkaloid, terpenoid or phenylpropanoid compounds, are disclosed. Also disclosed are compositions useful in such methods, e.g., a plant containing a recombinant nucleic acid that is effective for reducing the level of general DNA methylation.

### CROSS-REFERENCE TO RELATED APPLICATIONS

This application claims priority under 35 U.S.C. § 119 to U.S. Provisional Application Nos. 60/671,209, filed Apr. 14, 2005, and 60/733,588, filed Nov. 4, 2005, which are incorporated herein by reference in their entirety.

### TECHNICAL FIELD

This invention relates to methods and compositions for modulating the amount of one or more secondary metabolites in plants. In particular, the invention relates to methods and compositions for modulating the amount of one or more secondary metabolites in plants by manipulating the genome methylation status.

### INCORPORATION-BY-REFERENCE & TEXTS

The material on the accompanying diskette is hereby incorporated by reference into this application. The accompanying compact discs contain one file, 18207-005001.txt, which was created on Apr. 13, 2006. The file named 18207-005001.txt is 509 KB. The file can be accessed using Microsoft Word on a computer that uses Windows OS.

### BACKGROUND

Elucidating and manipulating an organism's temporal and spatial gene expression profile can be useful for developing new and improved biological products. Among the array of regulatory mechanisms that affect an organism's gene expression profile, the regulation of gene methylation has an important role. In many cases, gene methylation is regulated through site-specific methylation or demethylation of particular nucleotide sequences.

### SUMMARY

In one aspect, the invention features a method of producing one or more secondary metabolites which comprises extracting one or more secondary metabolites from plant cells. The cells contain a recombinant nucleic acid construct having a nucleic acid that modulates expression of a methylation status polypeptide and that is operably linked to a regulatory region that regulates transcription in the cells. The nucleic acid can be effective for modulating the expression of one or more genes involved in secondary metabolite biosynthesis. The secondary metabolite can be a terpenoid compound, an alkaloid compound, or a phenylpropanoid compound. The gene involved in secondary metabolite biosynthesis can code for an enzyme or regulatory protein involved in tetrahydrobenzylisoquinoline alkaloid biosynthesis, benzophenanthridine alkaloid biosynthesis, morphinan alkaloid biosynthesis, monoterpenoid indole alkaloid biosynthesis, bisbenzylisoquinoline alkaloid biosynthesis, pyridine, purine, tropane or quinoline alkaloid biosynthesis, terpenoid, betaine or phenethylamine alkaloid biosynthesis, or steroid alkaloid biosynthesis.

In some embodiments, the cells of the method are cells in tissue culture e.g., a rice tissue culture. In some embodiments, the cells are part of a whole plant, e.g., a rice plant. The regulatory region can confer constitutive transcription or selective transcription in inflorescences, embryos, or endosperm. The cells of the method can be a Papaveraceae tissue culture or a Papaveraceae plant. The regulatory region can confer constitutive transcription or selective transcription in laticifer cells, companion cells or sieve cells.

In another aspect, the invention features a method of producing a secondary metabolite, comprising growing plant cells that produce the secondary metabolite. The cells have a recombinant nucleic acid construct which includes a nucleic acid that modulates expression of a methylation status polypeptide, operably linked to a regulatory region that regulates transcription in the cells. Expression of the nucleic acid is effective for modulating the amount of one or more secondary metabolites in the cells.

The secondary metabolite can be an alkaloid compound, a terpenoid compound, or a phenylpropanoid compound. The terpenoid compound can be squalene, lupeol, α-amyrin, β-amyrin, glycyrrhizin, β-sitosterol, sitostanol, stigmasterol, α-tocopherol, β-tocopherol, γ-tocopherol, campesterol, ergosterol, diosgenin, aescin, picrotoxin, betulinic acid, asiaticoside, cucurbitacin E, glycyrrhizin, diosgenin, ruscogenin, lycopene, β-carotene, zeta-carotene, lutein, zeaxanthin, and antheraxanthin, phytoene, bixin, astaxanthin, yuanhuacin, yuanhuadin, glaucarubin, convallatoxin, squalamine, ouabain, or strophanthidin. The cells can be cells in tissue culture or part of a whole plant, e.g., a Taxus tissue culture or a Chrysanthemum, Tanacetum, Cinnamomum, Citrullus, Curcuma, Daphne, Euphorbia, Glycine, Glycyrrhiza, Gossypium, Guayule, Hevea, Lycopersicon, Mentha, Salvia, Rosmarinus, Simarouba, Artemisia, Taxus or Thymus plant.

The alkaloid compound can be salutaridine, salutaridinol, salutaridinol acetate, thebaine, isothebaine, papaverine, narcotine, narceine, noscapine, hydrastine, oripavine, morphinone, morphine, codeine, codeinone, and neopinone. Alternatively, the alkaloid compound can be berberine, palmatine, tetrahydropalmatine, S-canadine, columbamine, S-tetrahydrocolumbamine, S-scoulerine, S-cheilathifoline, S-stylopine, S-cis-N-methylstylopine, protopine, 6-hydroxyprotopine, R-norreticuline, S-norreticuline, R-reticuline, S-reticuline, 1,2-dehydroreticuline, S-3′-hydroxycoclaurine, S-norcoclaurine, S-coclaurine, S—N-methylcoclaurine, berbamunine, 2′-norberbamunine, laudanosine, or guatteguamerine. Alternatively, the alkaloid compound can be sanguinarine, dihydrosanguinarine, dihydroxy-dihydrosanguinarine, 12-hydroxy-dihydrochelirubine, 10-hydroxy-dihydrosanguinarine, dihydromacarpine, dihydrochelirubine, chelirubine, 12-hydroxychelirubine, or macarpine. The cells can be from a plant of the Papaveraceae, Menispermaceae, Lauraceae, Euphorbiaceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, or Rutaceae families, e.g., cells from a plant of the species Papaver bracteatum, Papaver orientale, Papaver setigerum, or Papaver somniferum. Alternatively, the cells can be from a plant of the species Croton salutaris, Croton balsamifera, Sinomenium acutum, Stephania cepharantha, Stephania zippeliana, Litsea sebiferea, Alseodaphne perakensis, Cocculus laurifolius, Duguetia obovata, Rhizocarya racemifera, or Beilschmiedia oreophila. Alternatively, the cells can be from a plant of the genera Sanguinaria, Dendromecon, Glaucium, Meconopsis, Chelidonium, Eschscholzia, or Argemone.

The method can further comprise the step of extracting the secondary metabolite from the plant.

In some embodiments, the regulatory region is a constitutive promoter, a tissue-specific promoter, or an inducible promoter. In some embodiments, the regulatory region confers transcription in laticifer cells, companion cells or sieve cells. A tissue-specific promoter can be a promoter specific for stem tissue, seed pod or parenchymal tissue.

The methylation status polypeptide in the above methods can be a cytosine DNA methyltransferase, or a decrease in DNA methylation polypeptide. The nucleic acid in the above methods can be an antisense or an interfering RNA to a cytosine DNA methyltransferase, or a decrease in DNA methylation polypeptide.

In another aspect, the invention features a transgenic plant that contains a recombinant nucleic acid construct. The construct comprises a nucleic acid that modulates expression of a methylation status polypeptide, operably linked to a regulatory region that regulates transcription in seeds. Expression of the nucleic acid is effective for modulating the amount of at least one secondary metabolite in a tissue of the plant relative to the amount in corresponding tissue from a control plant that lacks the recombinant nucleic acid construct, e.g., at least one of the secondary metabolites described herein. The amount of the secondary metabolite can be increased from about 1.5 fold to about 450 fold relative to the amount of the compound in the corresponding control plant. The amount of the secondary metabolite can be undetectable in the corresponding control plant.

In some embodiments, the plant is from the genera Aesculus, Anamirta, Andrographis, Artemisia, Betula, Bixa, Cannabis, Centella, Chrysanthemum, Cinnamomum, Citrullus, Coleus, Curcuma, Cymbopogan, Daphne, Euphorbia, Glycine, Glycyrrhiza, Gossypium, Guayule, Hevea, Isodon, Luffa, Mentha, Oryza, Rabdosia, Rosmarinus, Salvia, Simarouba, Tanacetum, Taxus, Thymus, or Tripterygium. In some embodiments, the plant is from the families Apocynaceae, Asclepiadaceae, Berberidaceae, Boraginaceae, Convolvulaceae, Euphorbiaceae, Erythroxylaceae, Gnetaceae, Lauraceae, Liliaceae, Menispermaceae, Papaveraceae, Leguminosae, Ranunculaeceae, Rubiaceae, Rutaceae, Solanaceae.

The methylation status polypeptide can be a cytosine DNA methyltransferase, or a decrease in DNA methylation polypeptide. The regulatory region can be a tissue-specific promoter, e.g., a promoter specific for stem tissue, seed pod or parenchymal tissue. The promoter can be a constitutive promoter, a tissue-specific promoter, or an inducible promoter.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows an alignment of cDNA ID 23631543, At MET1, (SEQ ID NO: 121) with orthologous amino acid sequences gi 37039880 (SEQ ID NO: 123); gi 2895089 (SEQ ID NO: 125); gi 2887280 (SEQ ID NO: 127); Ceres GDNA ANNOT ID no. 1478748 (SEQ ID NO: 129); gi 56130955 (SEQ ID NO: 131); and gi 3132825 (SEQ ID NO: 133), and a consensus sequence.

FIG. 2 shows an alignment of cDNA ID 23965502 RiceMET1 (SEQ ID NO: 135) with orthologous amino acid sequences gi 20977598 (SEQ ID NO: 137); gi 2895089 (SEQ ID NO: 139); gi 56130955 (SEQ ID NO: 141); gi 2887280 (SEQ ID NO: 143); and Ceres GDNA ANNOT ID no. 1478748 (SEQ ID NO: 145), and a consensus sequence.

FIG. 3 shows an alignment of cDNA ID 23505366 DDM1 (SEQ ID NO: 109) with orthologous amino acid sequences Ceres GDNA ANNOT ID no. 1533431 (SEQ ID NO: 111); gi 51536001 (SEQ ID NO: 113); gi 45357056 (SEQ ID NO: 115); gi 68144413 (SEQ ID NO: 117); and gi 37542688 (SEQ ID NO: 119), and a consensus sequence.

### DETAILED DESCRIPTION

The present invention is based on the discovery that an alteration in chromosomal 5′ cytosine methylation status in plants and plant cells results in modulation of the amount of one or more secondary metabolites in such plants and cells. This discovery has led to novel methods for modulating an amount of one or more secondary metabolites, compositions suitable for modulating the amount of one or more secondary metabolites, and compositions in which the amount of one or more secondary metabolites is modulated. Modulating the amount of one or more secondary metabolites in plants is useful, inter alia, to increase the yield of such compounds and for the discovery of new compounds.

I. Methods of Producing a Secondary Metabolite

In one aspect, the invention features a method of producing a secondary metabolite in a plant or plant cell. The method includes growing a plant or plant cell that has a recombinant nucleic acid construct. The recombinant nucleic acid construct includes a nucleic acid that modulates expression of a methylation status polypeptide and typically is operably linked to a regulatory region that drives transcription in the plant or plant cell. A methylation status polypeptide affects cytosine methylation status in genomic DNA. Without being bound by theory, it is believed that modulating expression of a methylation status polypeptide affects cytosine methylation status in chromatin and, via transcriptional gene activation and/or silencing, expression of one or more endogenous genes involved in secondary metabolite biosynthesis is altered, thereby modulating the amount and/or rate of biosynthesis of one or more secondary metabolites in a plant or plant cell. In this way, modulation of methylation status polypeptide expression modulates the amount of one or more secondary metabolites in the plant or plant cell.

Also provided herein are methods for producing a secondary metabolite in a plant or plant cell, by modulating the expression level of one or more endogenous genes involved in secondary metabolite biosynthesis. Such methods include growing a plant cell transformed with a recombinant nucleic acid construct that has a nucleic acid that modulates expression of a methylation status polypeptide, typically operably linked to a regulatory region that drives transcription in the plant or cell. One or more of the endogenous genes described herein can have its expression level modulated, e.g., increased or decreased, relative to the expression of the same endogenous gene in a corresponding plant or cell that is not transformed with the construct.

II. Methylation Status Polypeptides

Methods and compositions described herein utilize a nucleic acid that decreases expression of a methylation status polypeptide, i.e., a polypeptide that affects the pattern and/or relative level of cytosine methylation within genomic DNA, either generally or in a segment thereof. Such polypeptides are capable of affecting the methylation status of DNA in vivo and in vitro, and changes in their expression can be used to bring about changes in the methylation status of DNA. These polypeptides can affect the methylation status of genomic DNA, a segment or portion of genomic DNA, or a regulatory region. Polypeptides that affect methylation status are known to be present in a variety of organisms and are suitable for use in the methods described herein.

In some embodiments, such a polypeptide is a cytosine DNA methyltransferase. A number of methyltransferases (e.g., cytosine DNA methyltransferase) are known to catalyze the transfer of a methyl group to the C5 position of cytosine in DNA and play a role in the control of gene expression during development, including the polypeptide encoded by the Arabidopsis MET1 locus, the polypeptide encoded by the Arabidopsis MET2 locus, and orthologs thereof. See, e.g., SEQ ID NOS: 120-145, which describe orthologs and homologs of Arabidopsis and Oryza MET1, and nucleic acids encoding them.

In other embodiments, such a polypeptide is a decrease in DNA methylation 1 polypeptide (DDM1; SNF2 domain-containing proteins/helicase domain-containing proteins; e.g., At5g66750). See, e.g., SEQ ID NOS: 108-119, which describe orthologs and homologs of DDM1 and nucleic acids encoding them. The DDM1 polypeptide is found in the nucleosome, possesses an ATPase activity, and plays a role in methylation-dependent chromatin silencing.

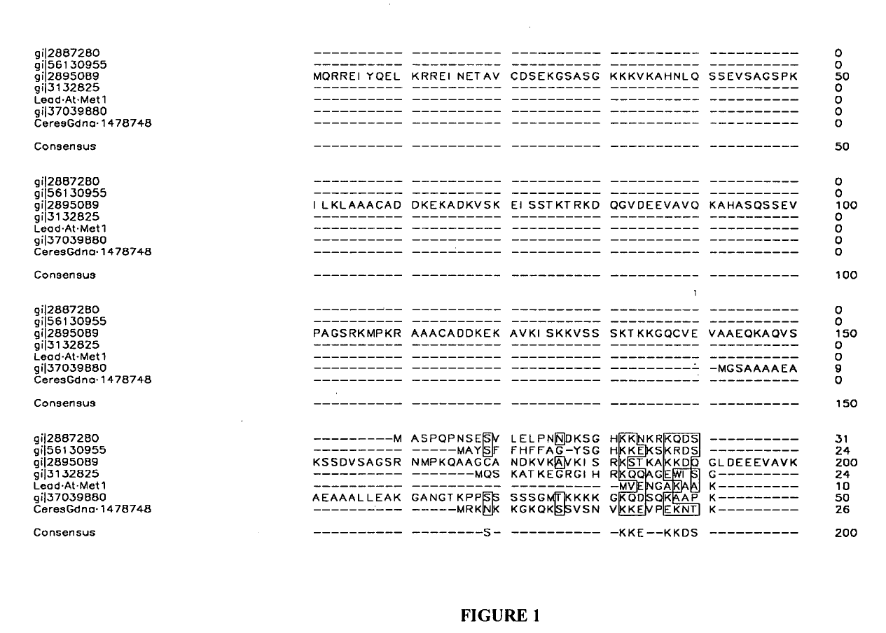

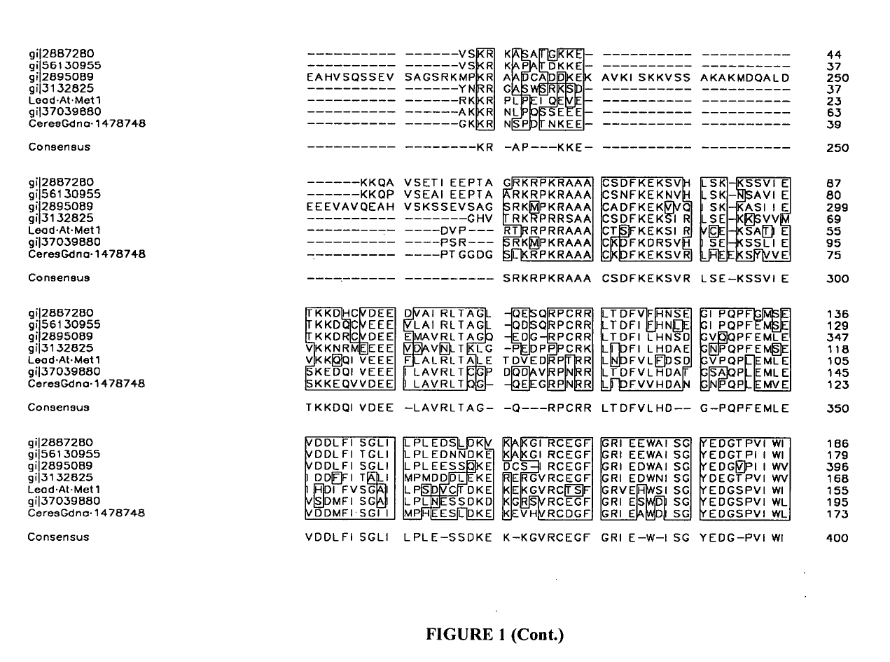

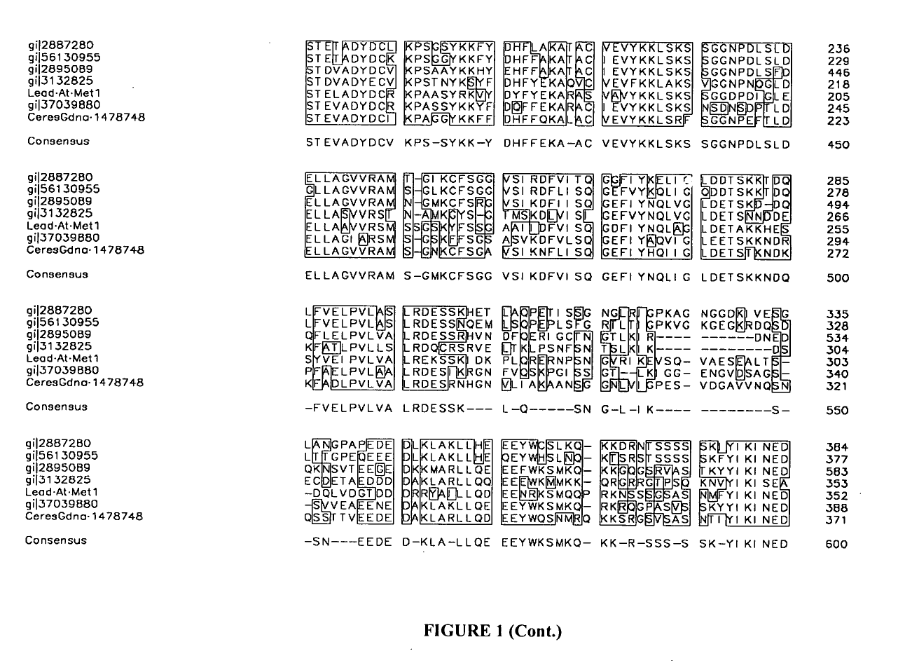

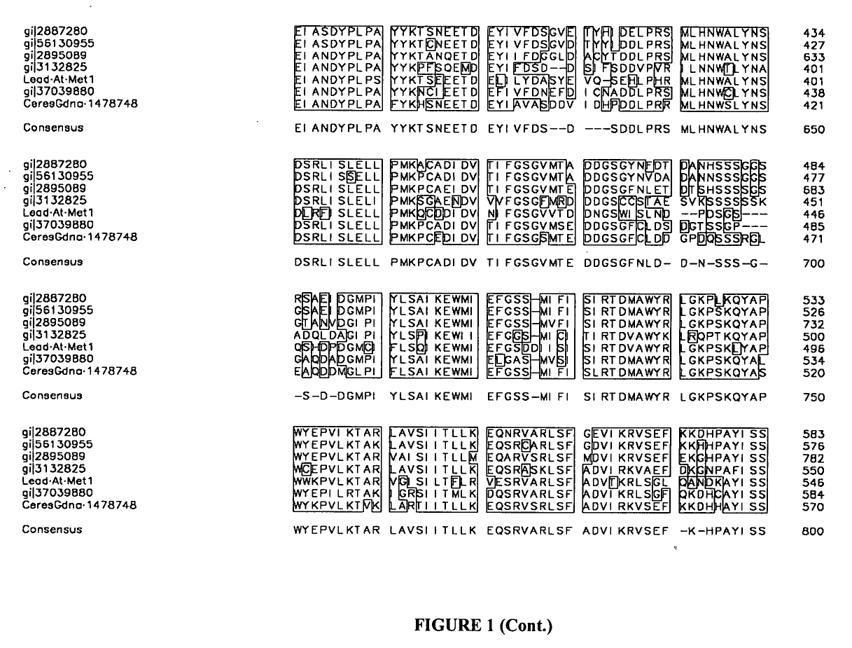

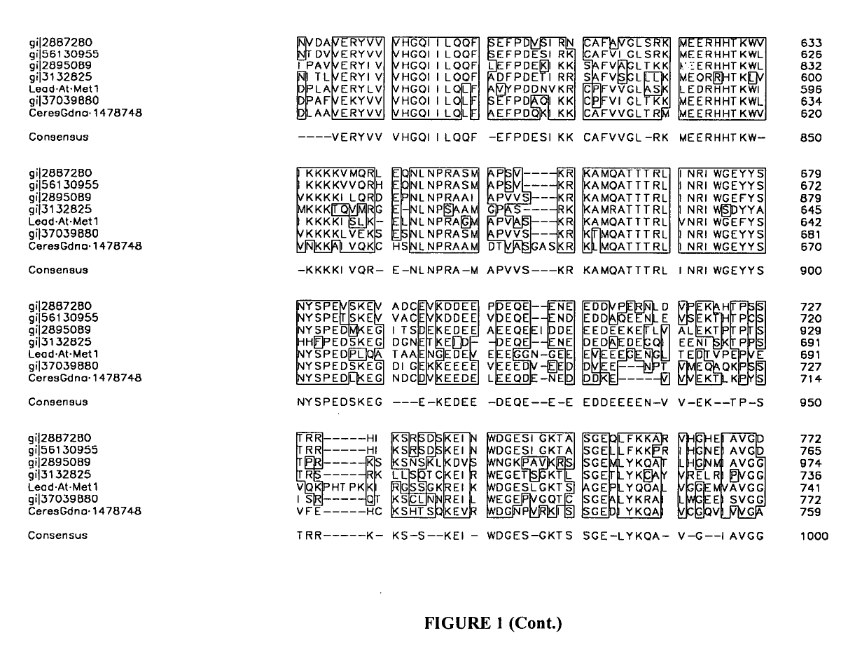

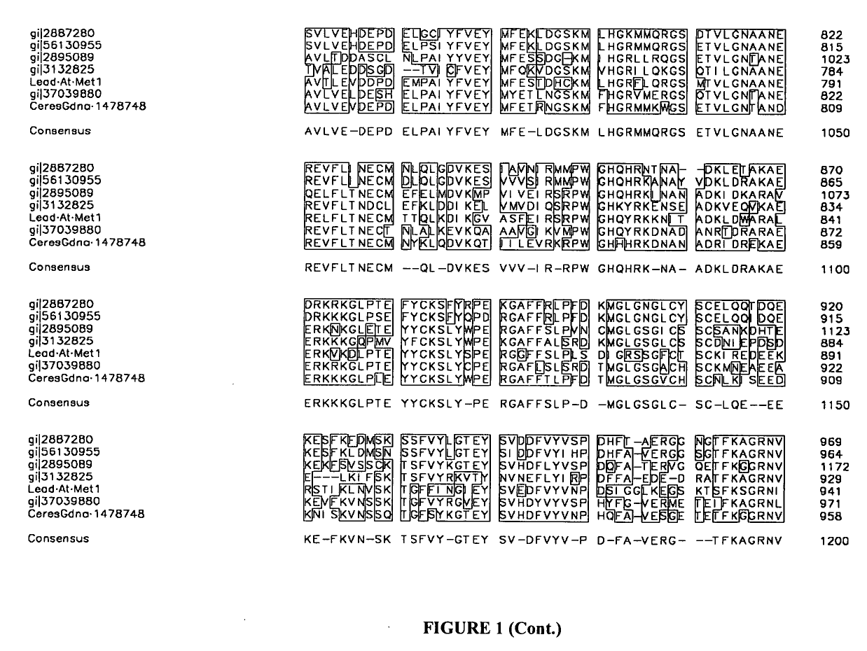

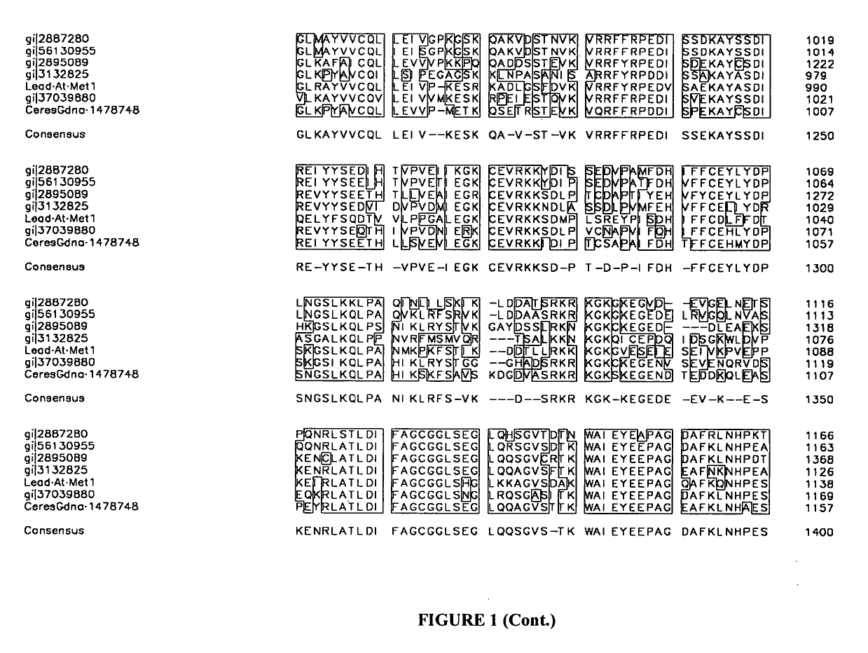

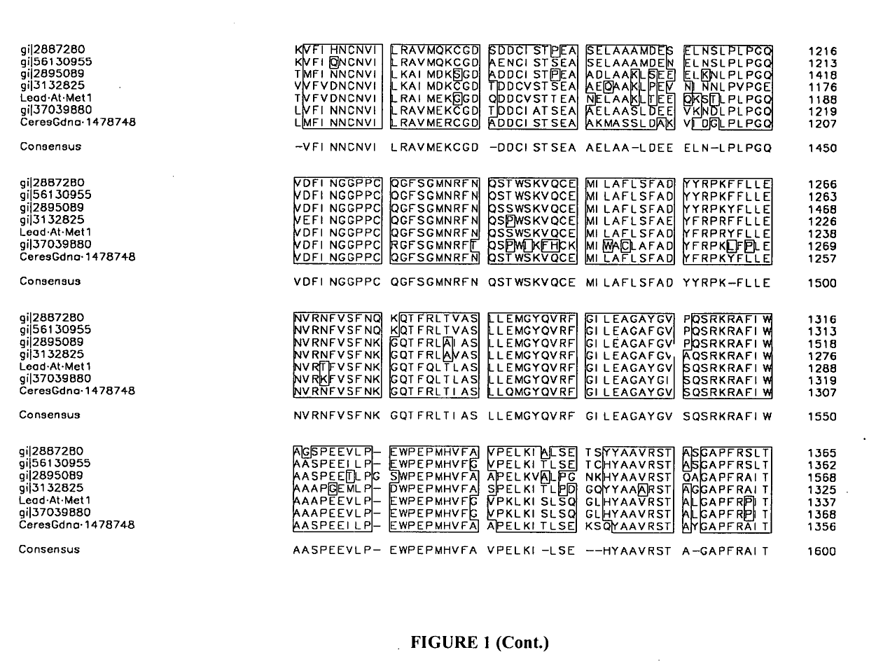

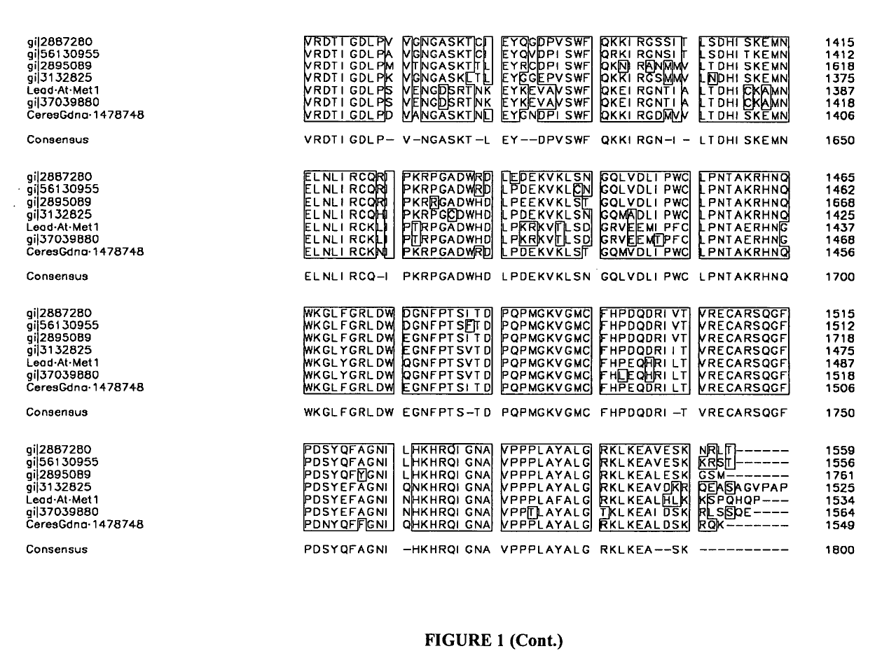

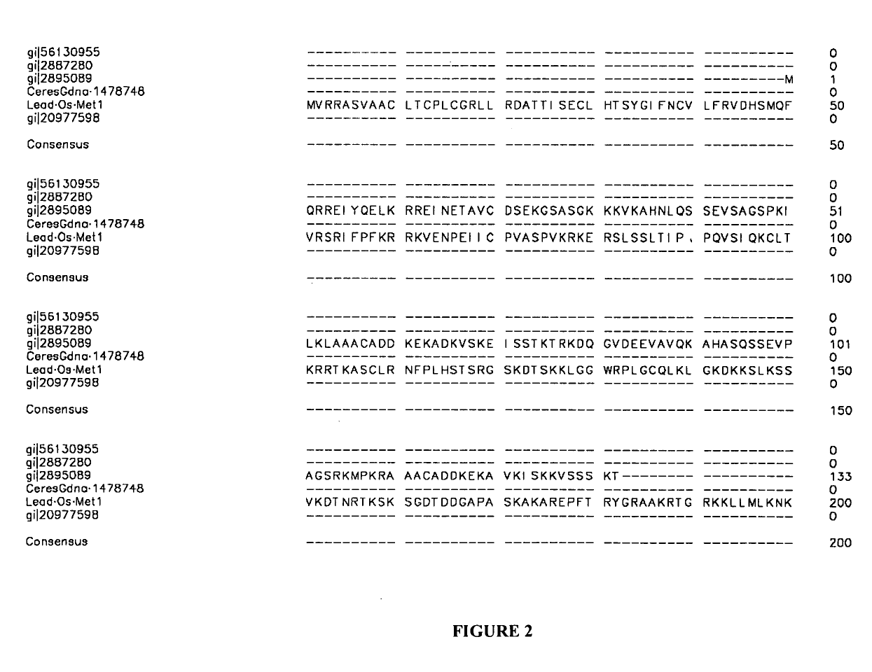

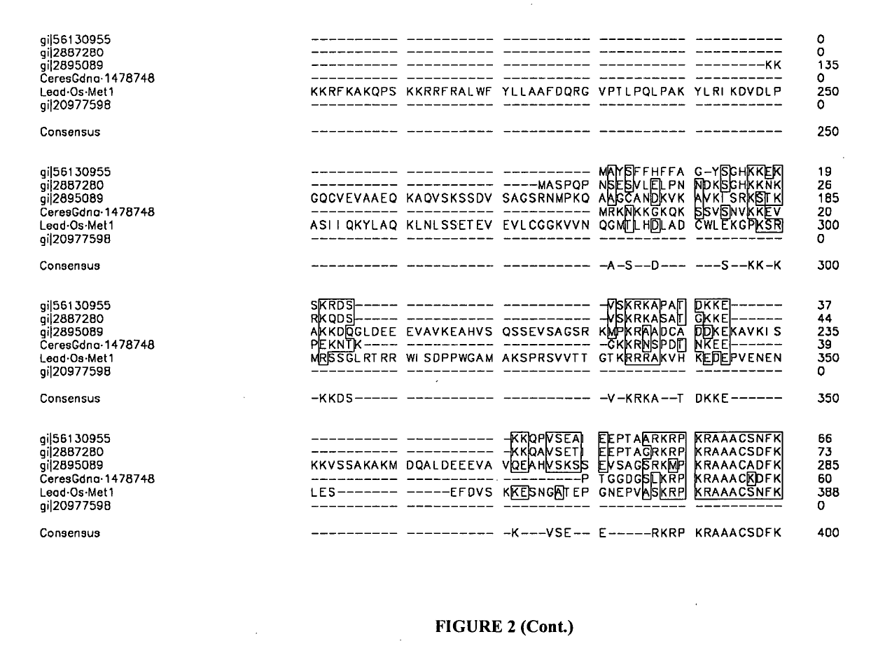

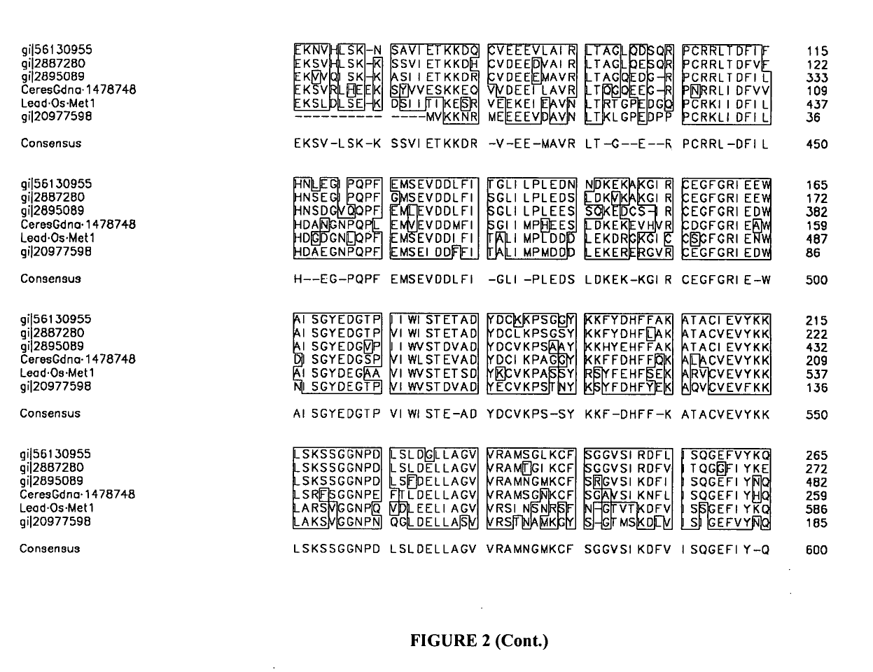

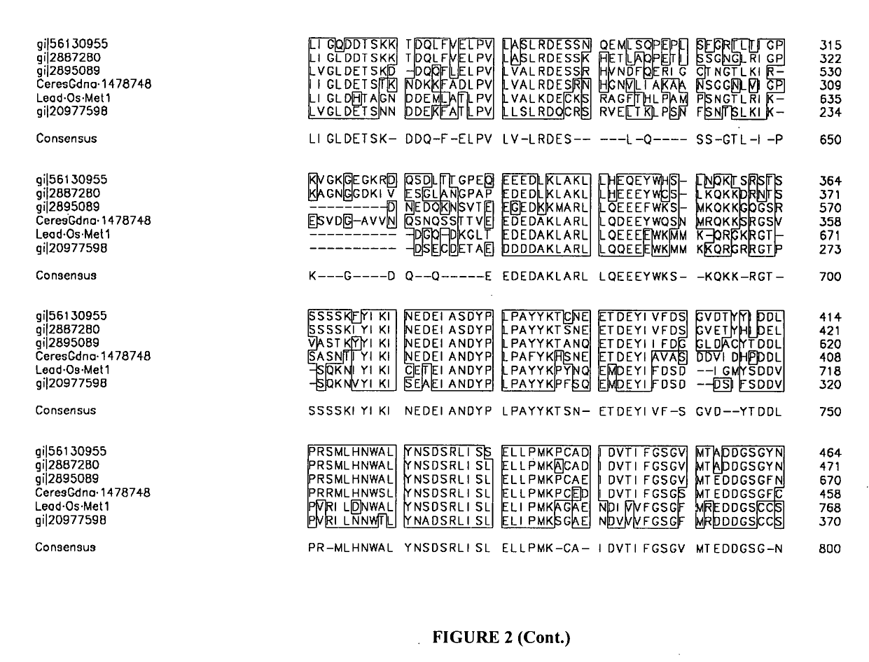

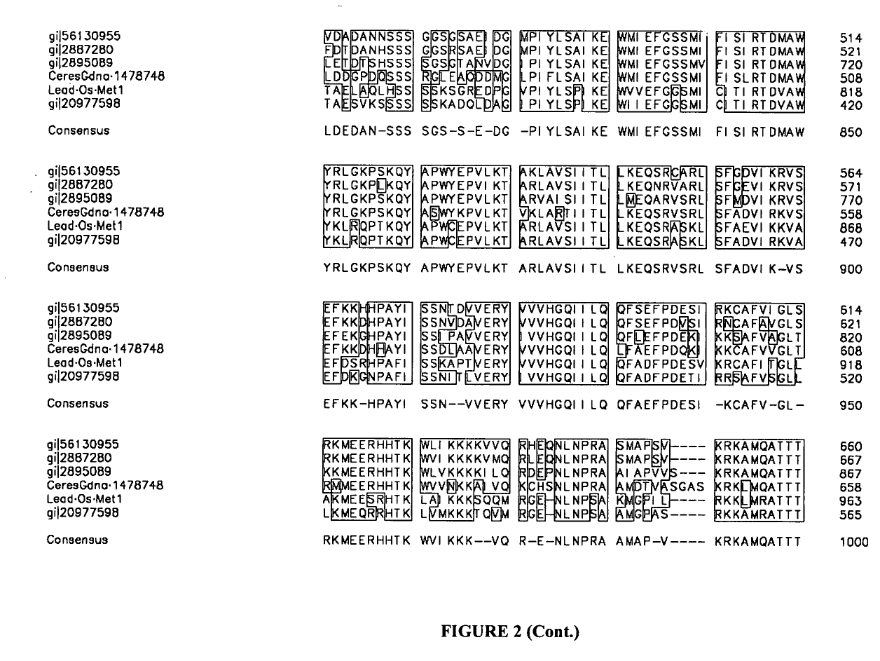

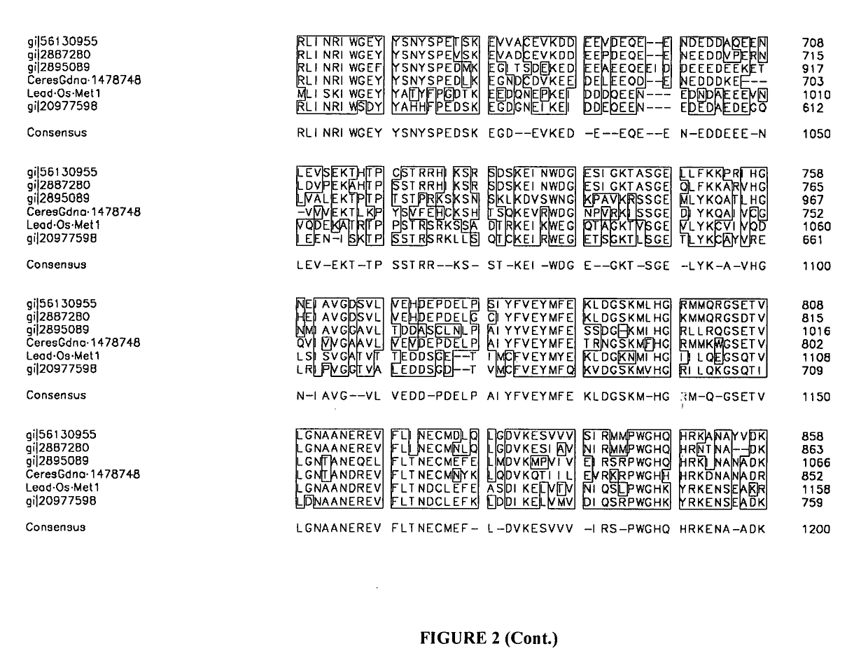

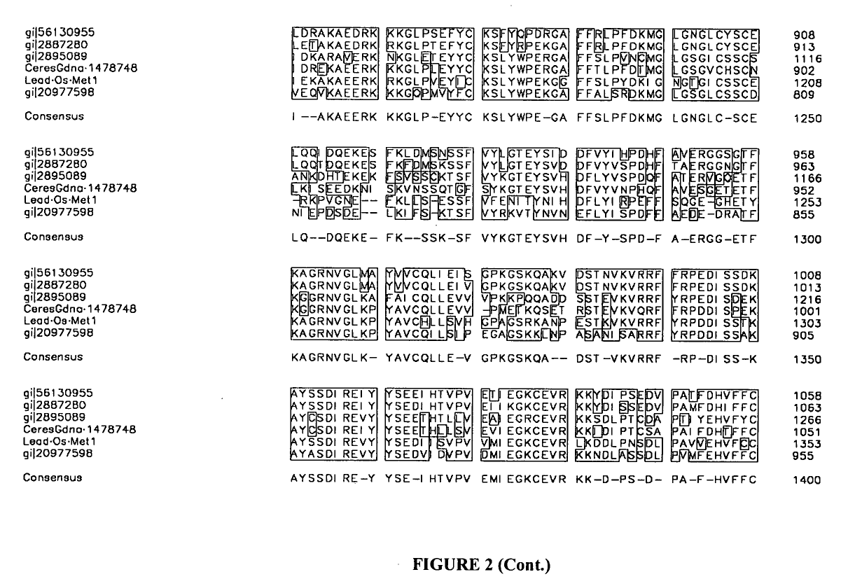

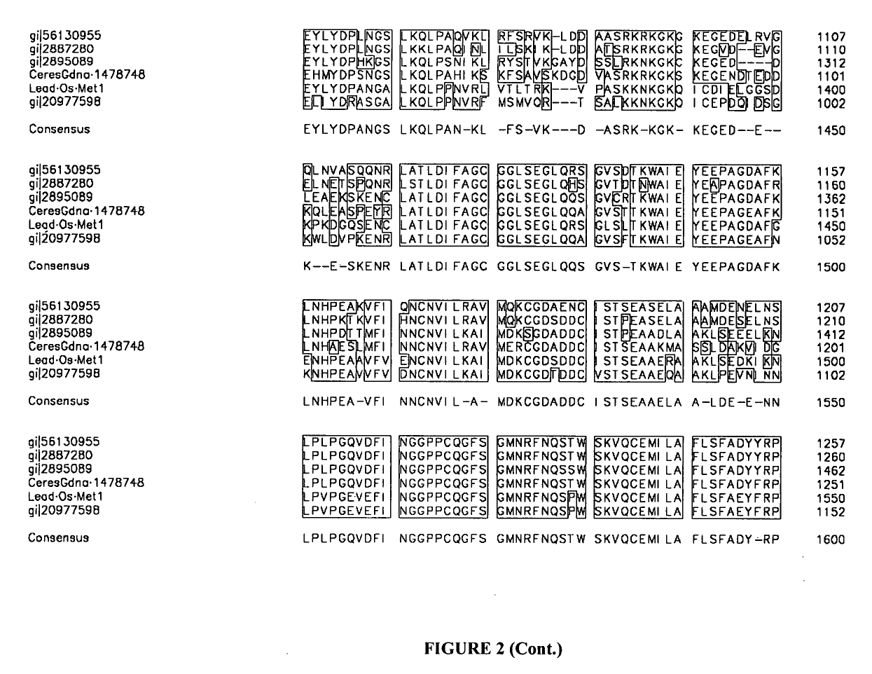

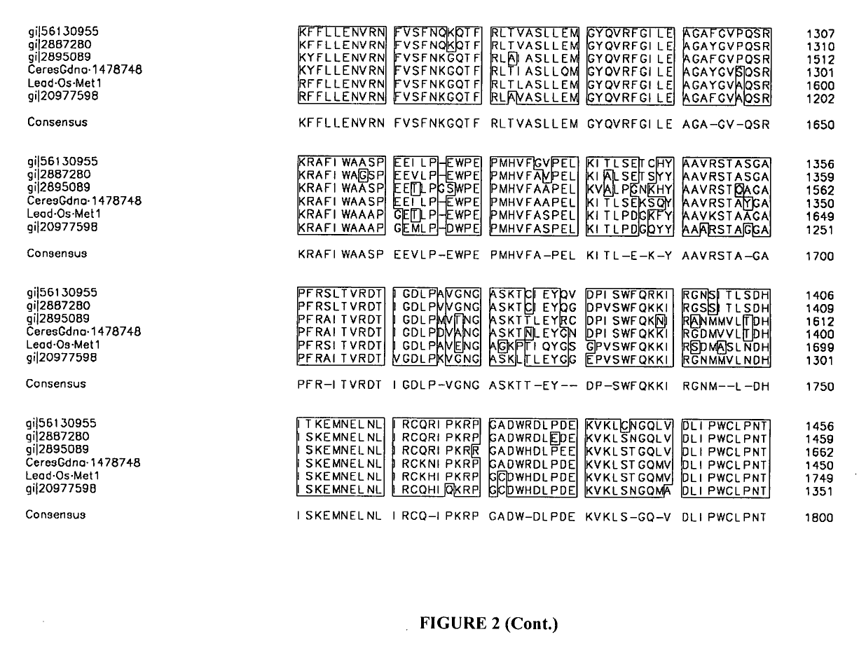

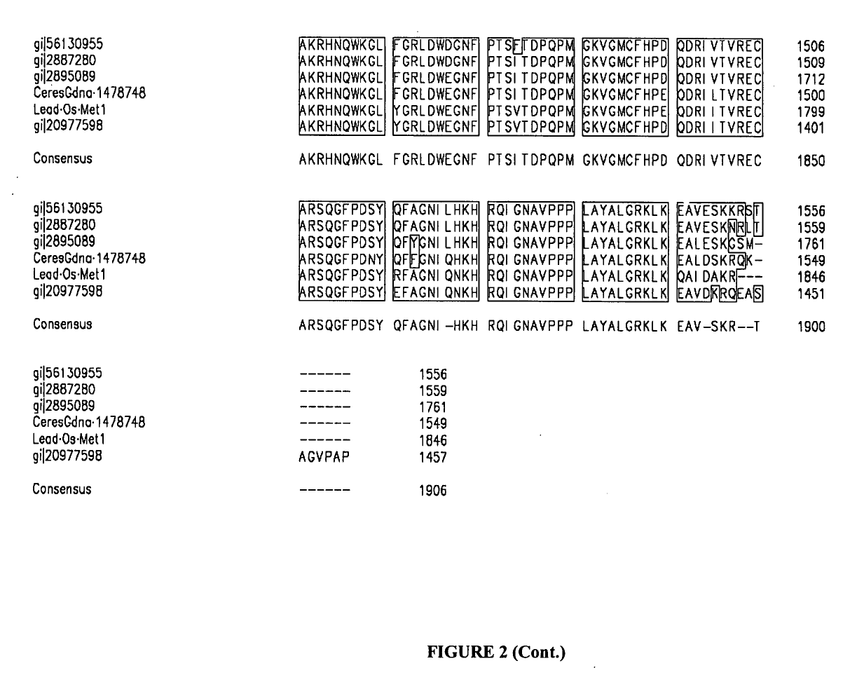

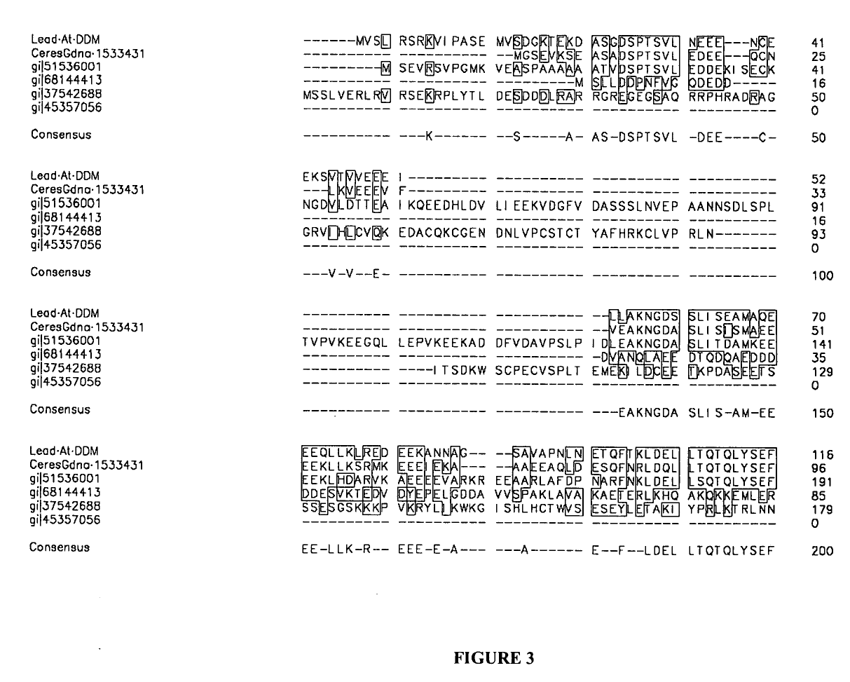

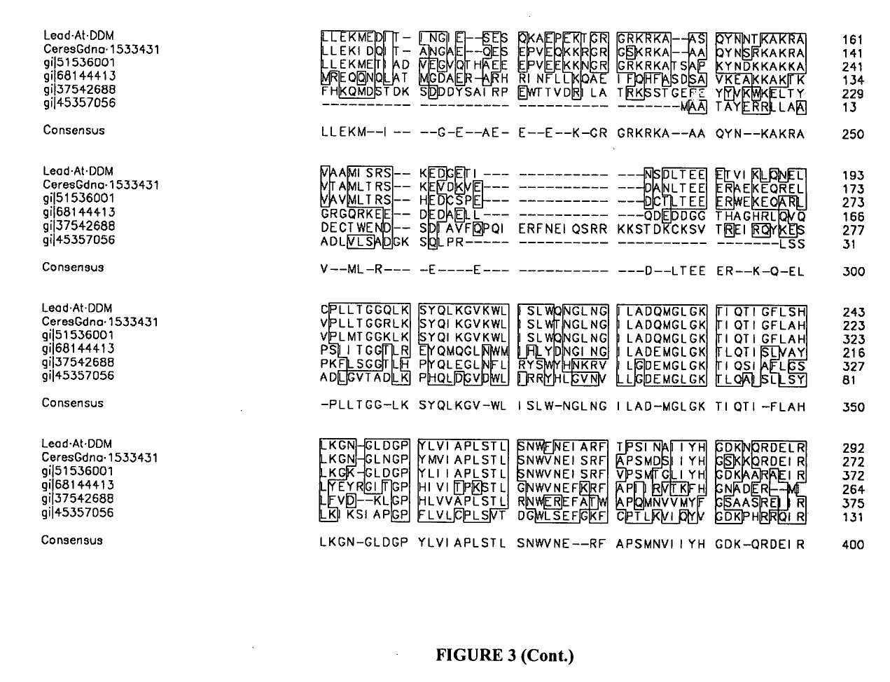

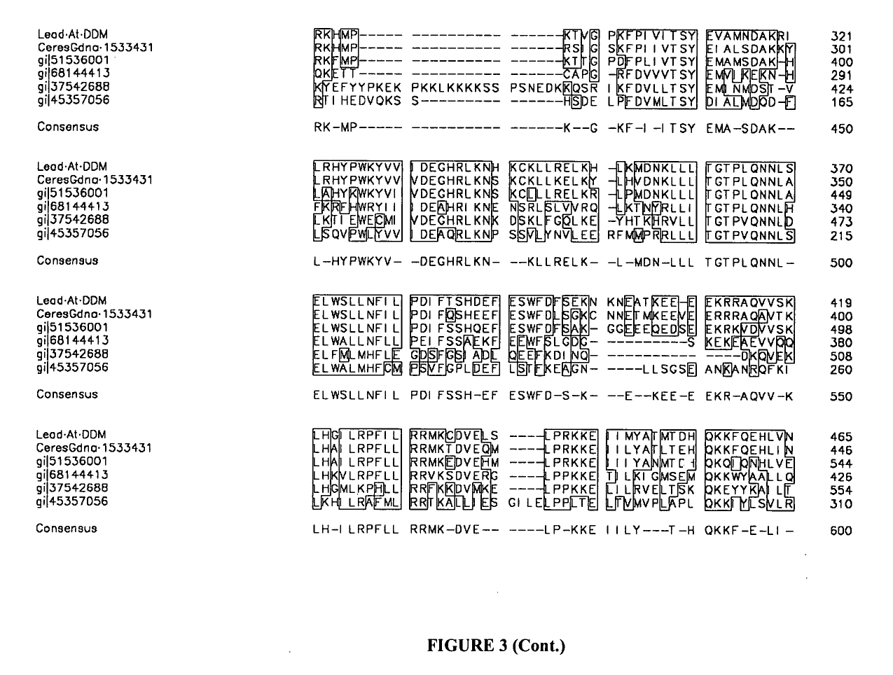

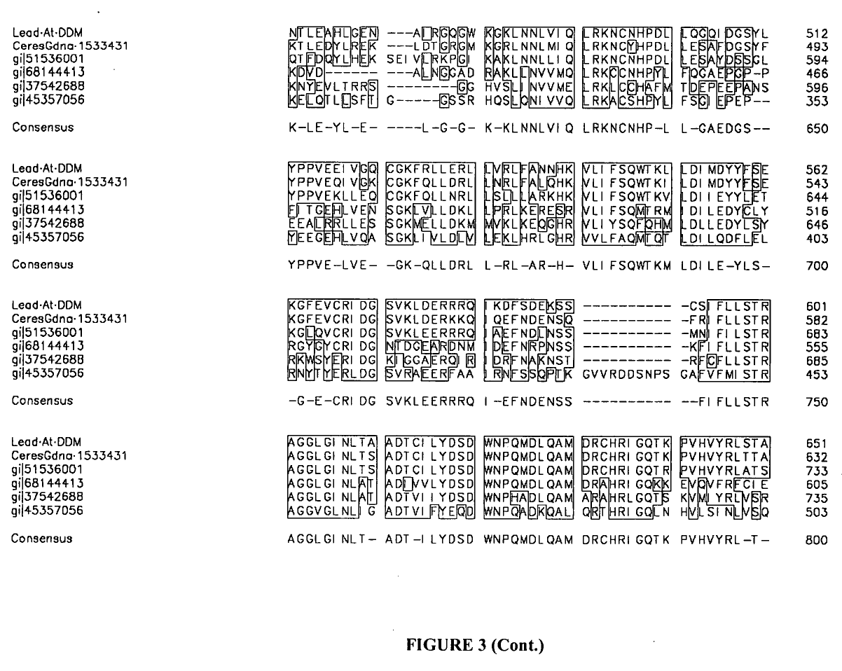

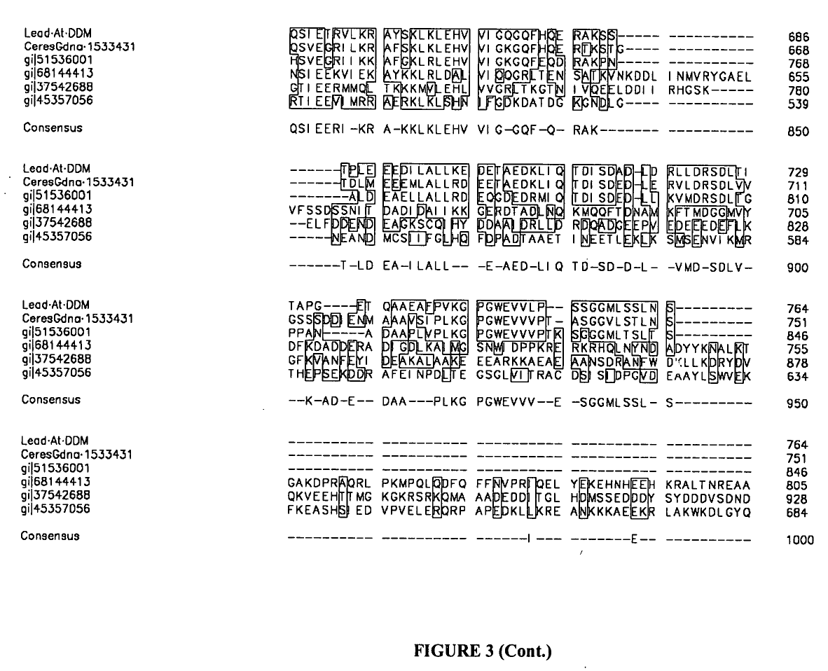

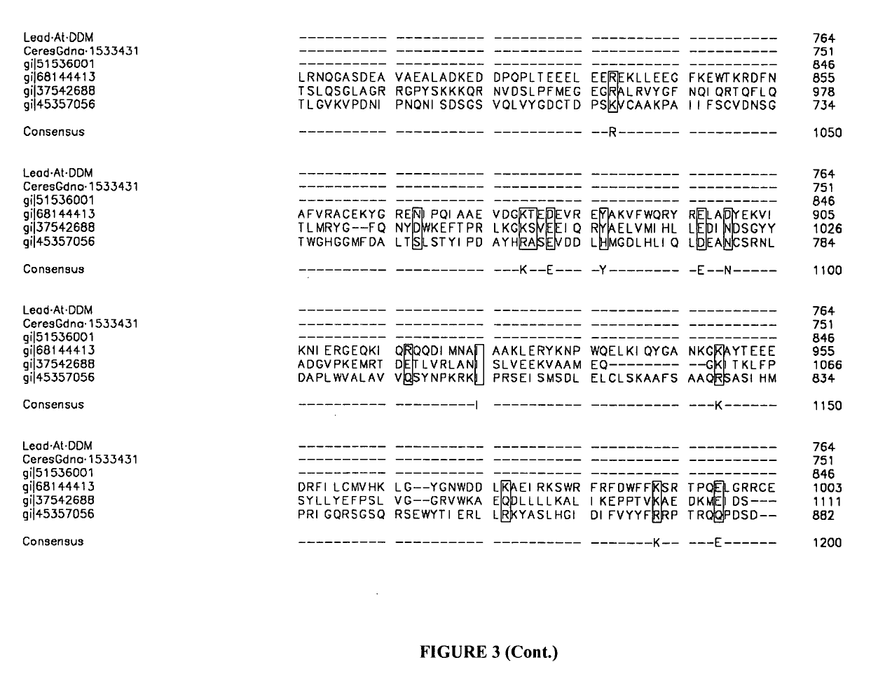

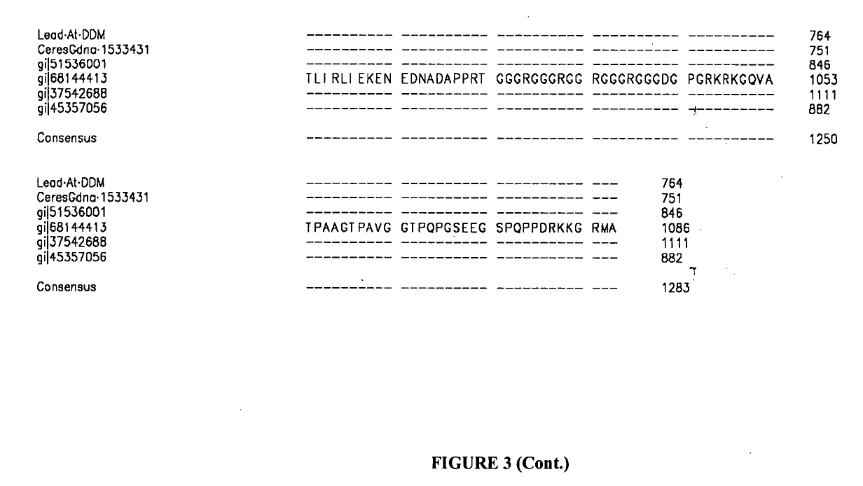

In some embodiments, a methylation status polypeptide is an ortholog, homolog or variant of the polypeptide having the amino acid sequence set forth in SEQ ID NO: 109, e.g., SEQ ID NOS: 111, 113, 115, 117, and 119, or the consensus sequence shown in FIG. 3. In some embodiments, a methylation status polypeptide is an ortholog, homolog or variant of the polypeptide having the amino acid sequence set forth in SEQ ID NO: 121, e.g., SEQ ID NOS: 123, 125, 127, 129, 131, and 133, or the consensus sequence shown in FIG. 1. In some embodiments, a methylation status polypeptide is an ortholog, homolog or variant of the polypeptide having the amino acid sequence set forth in SEQ IID NO: 135, e.g., SEQ ID NOS: 137, 139, 141, 143, and 145, or the consensus sequence shown in FIG. 2.

In certain cases, a methylation status polypeptide comprises an amino acid sequence having about 80% or greater sequence identity to SEQ ID NO: 121, 123, 125, 127, 129, 131, 133, 135, 137, 139, 141, 143, and 145. In some embodiments the percent sequence identity is about 82, 85, 87, 90, 92, 95, 96, 97, 98, 99, or greater percent sequence identity to such a sequence. In some embodiments, a methylation status polypeptide comprises an amino acid sequence having about 80% or greater sequence identity to SEQ ID NO: 109, 111, 113, 115, 117, and 119. In some embodiments the percent sequence identity is about 82, 85, 87, 90, 92, 95, 96, 97, 98, 99, or greater percent sequence identity to such a sequence.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chenna et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a “percent identity” between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that methods and compositions described herein may be able to utilize non-transgenic plant cells or plants that carry a mutation in a gene for a methylation status polypeptide. For example, a plant carrying a T-DNA insertion, a deletion, a transversion mutation, or a transition mutation in the coding sequence for one of the aforementioned methylation status polypeptides can affect cytosine methylation status in chromatin, and be used to produce one or more secondary metabolites.

Methylation status polypeptides that are suitable candidates for modulation can be identified in a variety of ways. For example, candidate methyltransferases can be screened to identify polypeptides that affect cytosine methylation by preparing nuclear extracts from axenic seedlings and incubating solubilized proteins from the extract with a hemi-methylated (CpI)ₙ substrate and radioactively labeled S-adenosyl-methionine. See, e.g., Kakutani et al., Nucleic Acids Res. 93:12406-12411 (1995).

Suitable methylation status polypeptides also can be identified by analysis of nucleotide and polypeptide sequence alignments. For example, performing a query on a database of nucleotide or polypeptide sequences can identify orthologs of the polypeptides having amino acid sequence set forth in SEQ ID NOS: 109, 121 and 135. Sequence analysis can involve BLAST or PSI-BLAST analysis of nonredundant databases using amino acid sequences of known methylation status polypeptides. Those proteins in the database that have greater than 40% sequence identity can be candidates for further evaluation for suitability as methylation status polypeptides. If desired, manual inspection of such candidates can be carried out in order to narrow the number of candidates to be further evaluated. Manual inspection can be performed by selecting those candidates that appear to have domains suspected of being present in methylation status polypeptides. A consensus amino acid sequence for a methylation status polypeptide can be determined by aligning amino acid sequences, e.g., amino acid sequences related to SEQ ID NO: 109, 121 and 135, from a variety of plant species and determining the most common amino acid or type of amino acid at each position. Consensus sequences are shown in FIGS. 1-3.

Typically, conserved regions of methylation status polypeptides exhibit at least 40% amino acid sequence identity (e.g., at least 45%, at least 50%, at least 60%, at least 70%, at least 80%, or at least 90% amino acid sequence identity). In some embodiments, a conserved region of target and template polypeptides exhibits at least 92%, 94%, 96%, 98%, or 99% amino acid sequence identity. Amino acid sequence identity can be deduced from amino acid or nucleotide sequences. In certain cases, highly conserved domains have been identified within methylation status polypeptides. These conserved regions can be useful in identifying functionally similar methylation status polypeptides.

Domains are groups of contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, each domain has been associated with either a conserved primary sequence or a sequence motif. Generally these conserved primary sequence motifs have been correlated with specific in vitro and/or in vivo activities. Examples of domains that can be used to identify orthologous cytosine DNA methyltransferases include, without limitation, a methyltransferase catalytic activity domain, a “eukaryotic” domain, a PWWP domain, an Ado-Met binding site, a TS domain, a bromo-adjacent homology (BAH) domain, a Cys-rich domain, a GK repeat domain, a UBA domain, and a PC repeat domain.

The identification of conserved regions in a template, or subject, polypeptide can facilitate production of variants of wild type methylation status polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing a variety of protein motifs and domains at sanger.ac.uk/Pfam and genome.wustl.edu/Pfam. A description of the information included at the Pfam database is described in Sonnhammer et al., 1998, Nucl. Acids Res. 26: 320-322; Sonnhammer et al., 1997, Proteins 28:405-420; and Bateman et al., 1999, Nucl. Acids Res. 27:260-262.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

In some embodiments, the amino acid sequence of a suitable subject polypeptide has greater than 40% sequence identity (e.g., >40%, >50%, >60%, >70% or >80%) to the amino acid sequence of the query polypeptide. In some embodiments, the nucleotide sequence of a suitable subject nucleic acid has greater than 70% sequence identity (e.g., >75%, >80%, >90%, >91%, >92%, >93%, >94%, >95%, >96%, >97%, >98%, or 25>99%) to the nucleotide sequence of the query nucleic acid. It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

If desired, the classification of a polypeptide as a methylation status polypeptide can be determined by techniques known to those having ordinary skill in the art. These techniques can be divided into two general categories: global methylation analysis, and gene-specific methylation analysis. Global methylation analysis techniques, such as chromatographic methods and a methyl accepting capacity assay, allow the measurement of the overall level of methyl cytosines in genomic DNA. One global methylation analysis technique includes digesting total genomic DNA with TaqI and labeling 5′ terminal cytosines in the digest with radioactivity. The labeled DNA is then digested to mononucleotides and the amount of methylated and unmethylated cytosine is estimated using thin layer chromatography. See, e.g., Kakutani, et al., Nucl. Acids Res. 93:12406-12411 (1995). In addition, techniques such as Restriction Landmark Genomic Scanning for Methylation (RLGS-M), and CpG island microarray can be used to identify unknown methylation hot-spots or methylated CpG islands in genomic DNA. Gene-specific methylation analysis techniques include the use of methylation sensitive restriction enzymes to digest DNA, followed by Southern detection or PCR amplification. For example, the methylation of single copy and repetitive sequences can be estimated from the digestion pattern observed in Southern blots of genomic DNA digested with HpaII or MspI. See, Jeddeloh et al., Plant J. 9:579-586 (1996) and Finnegan et al., Proc. Natl. Acad. Sci. USA 93:8449-8454 (1996). In addition, techniques based on bisulfite reaction are known, and include methylation specific PCR (MSP) and bisulfite genomic sequencing PCR. Other techniques include the use of hydrazine or potassium permanganate and ligation-mediated PCR.

III. Recombinant Nucleic Acid Constructs

A recombinant construct utilized in the methods and compositions described herein contains a nucleic acid that decreases the amount of transcription or translation product of a gene encoding a methylation status polypeptide, e.g., decreases the stability of, reduces the accumulation of, or decreases the translation of, an mRNA for such a polypeptide. Examples of nucleic acids that can affect expression of a methylation status polypeptide include antisense nucleic acids, ribozyme nucleic acids, or interfering RNA nucleic acids. Such nucleic acids are typically targeted in a plant or plant cell to a cytosine DNA methyltransferase or a DDM1 polypeptide.

In some embodiments, a nucleic acid that decreases the amount of transcription or translation product of a gene encoding a methylation status polypeptide is transcribed into an antisense nucleic acid or an interfering RNA similar or identical to the sense coding sequence of an ortholog, homolog or variant, e.g., SEQ ID NOS: 108, 110, 112, 114, 116, and 118. In some embodiments, a nucleic acid that decreases the amount of transcription or translation product of a gene encoding a methylation status polypeptide is transcribed into an antisense nucleic acid or an interfering RNA similar or identical to the sense coding sequence of an ortholog, homolog or variant, e.g., SEQ ID NOS: 120, 122, 124, 126, 128, 130, and 132. In some embodiments, a nucleic acid that decreases the amount of transcription or translation product of a gene encoding a methylation status polypeptide is transcribed into an antisense nucleic acid or an interfering RNA identical to all or part of the sense coding sequence of an ortholog, homolog or variant, e.g., SEQ ID NOS: 134, 136, 138, 140, 142, and 144. In such embodiments the antisense nucleic acid or interfering RNA is from about 15 nucleotides to about 2,500 nucleotides in length, or any integer therebetween as described herein. For example, the length of the antisense nucleic acid or interfering RNA nucleic acid can be 20 nucleotides, 21 nucleotides, 22 nucleotides, 23 nucleotides, 24 nucleotides, 25 nucleotides, 30 nucleotides, 35 nucleotides, 40 nucleotides, 50 nucleotides, 60 nucleotides, 75 nucleotides, 100 nucleotides, 200 nucleotides, 300 nucleotides, 400 nucleotides, 500 nucleotides, 1000 nucleotides, or 1500 nucleotides.

Thus, for example, a suitable nucleic acid can be an antisense nucleic acid to one of the aforementioned genes encoding a methylation status polypeptide. Alternatively, the transcription product of a nucleic acid can be similar or identical to the sense coding sequence of a methylation status polypeptide, but is an RNA that is unpolyadenylated, lacks a 5′ cap structure, or contains an unsplicable intron. In some embodiments, the nucleic acid is a partial or full-length coding sequence that, in sense orientation results in inhibition of the expression of an endogenous polypeptide by co-suppression. Methods of co-suppression using a full-length cDNA sequence as well as a partial cDNA sequence are known in the art. See, e.g., U.S. Pat. No. 5,231,020.

A suitable nucleic acid also can be transcribed into an interfering RNA. Such an RNA can be one that can anneal to itself, e.g., a double stranded RNA having a stem-loop structure. One strand of the stem portion of a double stranded RNA can comprise a sequence that is similar or identical to the sense coding sequence of an endogenous polypeptide, and that is from about 10 nucleotides to about 2,500 nucleotides in length. The length of the nucleic acid sequence that is similar or identical to the sense coding sequence can be from 15 nucleotides to 300 nucleotides, from 20 nucleotides to 100 nucleotides, or from 25 nucleotides to 100 nucleotides. The other strand of the stem portion of a double stranded RNA can comprise an antisense sequence of an endogenous polypeptide, and can have a length that is shorter, the same as, or longer than the length of the corresponding sense sequence. The loop portion of a double stranded RNA can be from 10 nucleotides to 500 nucleotides in length, e.g., from 15 nucleotides to 100 nucleotides, from 20 nucleotides to 300 nucleotides, or from 25 nucleotides to 400 nucleotides in length. The loop portion of the RNA can include an intron. See, e.g., WO 98/53083; WO 99/32619; WO 98/36083; WO 99/53050; and US patent publications 20040214330 and 20030180945. See also, U.S. Pat. Nos. 5,034,323; 6,452,067; 6,777,588; 6,573,099; and 6,326,527.

A suitable interfering RNA can be constructed as described in Brummell, et al., Plant J. 33:793-800 (2003). Examples of RNAi nucleic acids are shown in SEQ ID NOS: 104 and 106. SEQ ID NO: 104 comprises about 0.6 kb of a rice cytosine DNA methyltransferase sense strand (N-terminal region) and an inverted repeat of a nos terminator sequence. SEQ ID NO: 106 comprises about 0.7 kb of a rice cytosine DNA methyltransferase sense strand (C-terminal region) and an inverted repeat of a nos terminator sequence. Nucleic acid sequences for the N and C-terminal domains of the rice cytosine DNA methyltransferase are shown in SEQ ID NOS: 105 and 107, respectively.

As used herein, nucleic acid refers to RNA or DNA, and can be single- or double-stranded. If single-stranded, a nucleic acid having a polypeptide coding sequence can be either the coding or the non-coding strand.

A nucleic acid can be made by, for example, chemical synthesis or the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach, C. & Dveksler, G., Eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies are available by which site-specific nucleotide sequence modifications can be introduced into a template nucleic acid.

Nucleic acids can be detected by methods such as ethidium bromide staining of agarose gels, Southern or Northern blot hybridization, PCR or in situ hybridizations. Hybridization typically involves Southern or Northern blotting (see, for example, sections 9.37-9.52 of Sambrook et al., 1989, “Molecular Cloning, A Laboratory Manual”, 2^{nd} Edition, Cold Spring Harbor Press, Plainview; NY). Probes should hybridize under high stringency conditions to a nucleic acid or the complement thereof. High stringency conditions can include the use of low ionic strength and high temperature washes, for example 0.015 M NaCl/0.0015 M sodium citrate (0.1×SSC), 0.1% sodium dodecyl sulfate (SDS) at 65° C. In addition, denaturing agents, such as formamide, can be employed during high stringency hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

IV. Regulatory Regions

A recombinant nucleic acid construct disclosed herein typically includes one or more regulatory regions. The term “regulatory region” refers to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals and protease cleavage sites.

As used herein, the term “operably linked” refers to positioning of a regulatory region and a transcribable sequence in a nucleic acid so as to allow or facilitate transcription of the transcribable sequence. For example, to bring a coding sequence under the control of a promoter, it typically is necessary to position the translation initiation site of the translational reading frame of the coding sequence between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element. The choice of promoters to be included depends upon several factors, including, but not limited to, efficiency, selectability, inducibility, desired expression level, and cell or tissue specificity. It is a routine matter for one of skill in the art to modulate expression by appropriately selecting and positioning promoters and other regulatory regions relative to an operably linked sequence.

Some suitable promoters initiate transcription only, or predominantly, in certain cell types. For example, a promoter specific to a reproductive tissue (e.g., fruit, ovule, seed, pollen, pistils, female gametophyte, egg cell, central cell, nucellus, suspensor, synergid cell, flowers, embryonic tissue, embryo, zygote, endosperm, integument, seed coat or pollen) is used. A cell type or tissue-specific promoter may drive expression of operably linked sequences in tissues other than the target tissue. Thus, as used herein a cell type or tissue-specific promoter is one that drives expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

Examples of various classes of promoters are described below. Some of the promoters indicated below as well as additional promoters are described in more detail in U.S. Patent Application Ser. Nos. 60/505,689; 60/518,075; 60/544,771; 60/558,869; 60/583,691; 60/619,181; 60/637,140; 60/757,544; 60/776,307; Ser. Nos. 10/957,569; 11/058,689; 11/172,703; 11/208,308; 11/274,890; and PCT/US05/23639. Nucleotide sequences of promoters are set forth in SEQ ID NOS: 1-103. It will be appreciated that a promoter may meet criteria for one classification based on its activity in one plant species, and yet meet criteria for a different classification based on its activity in another plant species.

Constitutive Promoters

Constitutive promoters can promote transcription of an operably linked nucleic acid under most, but not necessarily all, environmental conditions and states of development or cell differentiation. Non-limiting examples of constitutive promoters that can be included in the nucleic acid constructs provided herein include the cauliflower mosaic virus (CaMV) 35S transcription initiation region, the mannopine synthase (MAS) promoter, the 1′ or 2′ promoters derived from T-DNA of Agrobacterium tumefaciens, the figwort mosaic virus 35S promoter, actin promoters such as the rice actin promoter, and ubiquitin promoters such as the maize ubiquitin-1 promoter.

Broadly Expressing Promoters

A promoter can be said to be “broadly expressing” when it promotes transcription in many, but not all, plant tissues. For example, a broadly expressing promoter can promote transcription of an operably linked sequence in one or more of the stem, shoot, shoot tip (apex), and leaves, but weakly or not at all in tissues such as reproductive tissues of flowers and developing seeds. In certain cases, a broadly expressing promoter operably linked to a sequence can promote transcription of the linked sequence in a plant shoot at a level that is at least two times, e.g., at least 3, 5, 10, or 20 times, greater than the level of transcription in a developing seed. In other cases, a broadly expressing promoter can promote transcription in a plant shoot at a level that is at least two times, e.g., at least 3, 5, 10, or 20 times, greater than the level of transcription in a reproductive tissue of a flower. In view of the above, the CaMV 35S promoter is not considered a broadly expressing promoter. Non-limiting examples of broadly expressing promoters that can be included in the nucleic acid constructs provided herein include the p326, YP0158, YP0214, YP0380, PT0848, PT0633, YP0050, YP0144 and YP0190 promoters (SEQ ID NOS: 76, 57, 61, 70, 26, 7, 35, 55, and 59, respectively).

Root-Specific Promoters

Root-specific promoters confer transcription only or predominantly in root tissue, e.g., root endodermis, root epidermis or root vascular tissues. Root-specific promoters include the YP0128, YP0275, PT0625, PT0660, PT0683 and PT0758 promoters (SEQ ID NOS: 52, 63, 6, 9, 14, and 22, respectively). Other root-specific promoters include the PT0613, PT0672, PT0678, PT0688 and PT0837 promoters (SEQ ID NOS: 5, 11, 13, 15, and 24, respectively), which drive transcription primarily in root tissue and to a lesser extent in ovules and/or seeds. Promoter p32449 (SEQ ID NO: 77) has preferential activity in roots, and somewhat less activity in other vegetative tissues. Other examples of root-specific promoters include the root specific subdomains of the CaMV 35S promoter (Lam et al., Proc Natl Acad Sci USA 86:7890-7894 (1989)), root cell specific promoters reported by Conkling et al. Plant Physiol. 93:1203-1121 (1990), and the tobacco RD2 gene promoter.

Maturing Endosperm Promoters

In some embodiments, promoters that preferentially drive transcription in maturing endosperm can be useful. Transcription from a maturing endosperm-specific promoter typically begins after fertilization and occurs primarily in endosperm tissue during seed development and is typically highest during the cellularization phase. Non-limiting examples of maturing endosperm-specific promoters that can be included in the nucleic acid constructs provided herein include the napin promoter, the Arcelin-5 promoter, the phaseolin gene promoter (Bustos et al., Plant Cell 1(9):839-853 (1989)), the soybean trypsin inhibitor promoter (Riggs et al., Plant Cell 1(6):609-621 (1989)), the ACP promoter (Baerson et al., Plant Mol Biol, 22(2):255-267 (1993)), the stearoyl-ACP desaturase gene (Slocombe et al., Plant Physiol 104(4):167-176 (1994)), the soybean α subunit of β-conglycinin promoter (Chen et al., Proc Natl Acad Sci USA 83:8560-8564 (1986)), the oleosin promoter (Hong et al., Plant Mol Biol 34(3):549-555 (1997)), zein promoters such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell Biol. 13:5829-5842 (1993)), the β-amylase gene promoter, and the barley hordein gene promoter. Other maturing endosperm-specific promoters include the YP0092, PT0676 and PT0708 promoters (SEQ ID NOS: 38, 12, and 17, respectively).

Ovary Tissue Promoters

Promoters that are active in ovary tissues such as the ovule wall and mesocarp can also be useful, e.g., a polygalacturonidase promoter or banana TRX promoter. Examples of promoters that are active primarily in ovules include YP0007, YP0111, YP0092, YP0103, YP0028, YP0121, YP0008, YP0039, YP0115, YP0119, YP0120, and YP0374 (SEQ ID NOS: 30, 46, 38, 43, 33, 51, 31, 34, 47, 49, 50, and 68, respectively).

Embryo Sac/Early Endosperm Promoters

To achieve embryo sac/early endosperm specific expression, regulatory regions can be used that preferentially drive transcription in polar nuclei and/or the central cell, or in precursors to polar nuclei, but not in egg cells or precursors to egg cells. A pattern of transcription that extends from polar nuclei into early endosperm development can also be found with embryo sac/early endosperm specific promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, Genbank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038); Arabidopsis viviparous-1 (see, Genbank No. U93215); Arabidopsis atmycl (see, Urao (1996) Plant Mol. Biol., 32:571-57; Conceicao (1994) Plant, 5:493-505). Other promoters include the following Arabidopsis promoters: YP0039, YP0101, YP0102, YP0110, YP0117, YP0119, YP0137, DME, YP0285 and YP0212 (SEQ ID NOS: 34, 41, 42, 45, 48, 49, 53, 88, 64, and 60, respectively). Other promoters that may be useful include the following rice promoters: p530c10, pOsFIE2-2, pOsMEA, pOsYp102, and pOsYp285, having SEQ ID NOS: 97, 90, 91, 92, and 93, respectively.

Embryo-Specific Promoters

Regulatory regions that preferentially drive transcription in zygotic cells following fertilization can provide embryo-specific expression. Most suitable are promoters that preferentially drive transcription in early stage embryos prior to the heart stage, but expression in late stage and maturing embryos is also suitable. Embryo-specific promoters include the barley lipid transfer protein (Ltp1) promoter (Plant Cell Rep (2001) 20:647-654), YP0097, YP0107, YP0088, YP0143, YP0156, PT0650, PT0695, PT0723, PT0838, PT0879, and PT0740 (SEQ ID NOS: 40, 44, 37, 54, 56, 8, 16, 19, 25, 28, and 20, respectively).

Photosynthetically-Active Tissue Promoters

Photosynthetically-active tissue promoters confer transcription only or predominantly in photosynthetically active tissue. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol. 35:773-778 (1994)), the Cab-1 gene promoter from wheat (Fejes et al., Plant Mol. Biol. 15:921-932 (1990)), the CAB-1 promoter from spinach (Lubberstedt et al., Plant Physiol. 104:997-1006 (1994)), the cab1R promoter from rice (Luan et al., Plant Cell 4:971-981 (1992)), the pyruvate, orthophosphate dikinase (PPDK) promoter from corn (Matsuoka et al., Proc Natl Aca. Sci USA 90:9586-9590 (1993)), the tobacco Lhcb1*2 promoter (Cerdan et al., Plant Mol. Biol. 33:245-255 (1997)), the Arabidopsis thaliana SUC2 sucrose-H+ symporter promoter (Truernit et al., Planta. 196:564-570 (1995)), and thylakoid membrane protein promoters from spinach (psaD, psaF, psaE, PC, FNR, atpC, atpD, cab, rbcS). Other photosynthetic tissue promoters include PT0535, PT0668, PT0886, YP0144, YP0380, and PT0585 (SEQ ID NOS: 3, 2, 29, 55, 70, and 4, respectively).

Vascular Tissue Promoters

Examples of promoters that have high or preferential activity in vascular bundles include YP0087 (SEQ ID NO: 83), YP0093 (SEQ ID NO: 84), YP0108 (SEQ ID NO: 85), YP0022 (SEQ ID NO: 81), and YP0080 (SEQ ID NO:82). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2): 185-192 (1992)), and the rice tungro bacilliform virus (RTBV) promoter (Dai et al., Proc. Natl. Acad. Sci. USA, 101(2):687-692 (2004)).

Poppy Capsule Promoters

Examples of promoters that have high or preferential activity in siliques/fruits, which are botanically equivalent to capsules in opium poppy, include PT0565 (SEQ ID NO: 79) and YP0015 (SEQ ID NO: 80).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO: 70), PT0848 (SEQ ID NO: 26), YP0381 (SEQ ID NO: 71), YP0337 (SEQ ID NO: 66), PT0633 (SEQ ID NO: 7), YP0374 (SEQ ID NO: 68), PT0710 (SEQ ID NO: 18), YP0356 (SEQ ID NO: 67), YP0385 (SEQ ID NO: 73), YP0396 (SEQ ID NO: 74), YP0388, YP0384 (SEQ ID NO: 72), PT0688 (SEQ ID NO: 15), YP0286 (SEQ ID NO: 65), YP0377 (SEQ ID NO: 69), PD1367 (SEQ ID NO: 78), PD0901 (SEQ ID NO: 87), and PD0898 (SEQ ID NO: 86). Nitrogen-inducible promoters include PT0863 (SEQ ID NO: 27), PT0829 (SEQ ID NO: 23), PT0665 (SEQ ID NO: 10), and PT0886 (SEQ ID NO: 29).

Laticifer- and Sieve-Preferential Promoters

Other useful promoters are those promoters that preferentially drive transcription in laticifer cells (found in the cortical cells of the stem), sieve elements, or companion cells; see, e.g., Bird et al., The Plant Cell, 15:2626-2635 (2003).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, inducible promoters, such as promoters that confer transcription in response to external stimuli such as chemical agents, developmental stimuli, or environmental stimuli. Promoters designated p13879, YP0086, YP0188, YP0263, PT0758, PT0743, PT0829, YP0119 and YP0096 (SEQ ID NOS: 75, 36, 58, 62, 22, 21, 23, 49, and 39, respectively), as described in the above-referenced patent applications, may be useful.

Other Regulatory Regions

A 5′ untranslated region (UTR) is transcribed, but is not translated, and lies between the start site of the transcript and the translation initiation codon and may include the +1 nucleotide. See, e.g., PCT publication WO 03/025172. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences, e.g., a nopaline synthase termination sequence.

A suitable enhancer is a cis-regulatory element (−212 to −154) from the upstream region of the octopine synthase (ocs) gene. Fromm et al., The Plant Cell 1:977-984 (1989).

Recombinant nucleic acid constructs provided herein also can include, for example, origins of replication, scaffold attachment regions (SARs), and/or markers. A marker gene can confer a selectable phenotype on a plant cell. For example, a marker can confer, biocide resistance, such as resistance to an antibiotic (e.g., kanamycin, G418, bleomycin, or hygromycin), or a herbicide (e.g., glyphosate, chlorosulfuron, glufosinate, or phosphinothricin). In addition, a recombinant nucleic acid construct can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

It will be understood that more than one regulatory region may be present in a recombinant polynucleotide, e.g., introns, enhancers, upstream activation regions, and inducible elements. Thus, more than one regulatory region can be operably linked to the sequence for a methylation status polypeptide.

V. Transgenic Plants and Cells A plant or plant cell used in methods and compositions of the invention contains one or more recombinant nucleic acid constructs as described herein. The plant or plant cell can be transformed and have the construct integrated into its genome, i.e., be stably transformed. Stably transformed cells typically retain the introduced nucleic acid sequence with each cell division. The plant or plant cells can also be transformed and have the construct not integrated into its genome. Such transformed cells are called transiently transformed cells. Transiently transformed cells typically lose all or some portion of the introduced nucleic acid construct with each cell division such that the introduced nucleic acid cannot be detected in daughter cells after sufficient number of cell divisions.

Typically, transgenic plant cells used in methods described herein constitute part or all of a whole plant. Such plants can be grown in a manner suitable for the species under consideration, either in a growth chamber, a greenhouse, or in a field. Fertile transgenic plants can be bred as desired for a particular purpose, e.g., to introduce a recombinant nucleic acid into other lines, to transfer a recombinant nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. Seeds produced by a transgenic plant can be grown and then selfed (or outcrossed and selfed) to obtain seeds homozygous for the nucleic acid construct.

In other embodiments, transgenic plant cells are grown in suspension culture, or tissue or organ culture, for production of secondary metabolites. For the purposes of this invention, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

Techniques for introducing exogenous nucleic acids into monocotyledonous, dicotyledonous, and gymnosperm plants are known in the art, and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or tissue culture is used as the recipient tissue for transformation, plants can be regenerated from transformed cultures if desired, by techniques known to those skilled in the art. See, e.g., Allen et al., “RNAi-mediated replacement of morphine with the normarcotic alkaloid reticuline in opium poppy,” Nature Biotechnology 22(12):1559-1566 (2004); Chitty et al., “Genetic transformation in commercial Tasmanian cultures of opium poppy, Papaver somniferum, and movement of transgenic pollen in the field,” Funct. Plant Biol. 30:1045-1058 (2003); and Park et al., J. Exp. Botany 51(347):1005-1016 (2000). See also, WO99/34663. Methods of making a transgenic plant include the step of introducing the desired nucleic acid into a recipient tissue, identifying transformants in the recipient tissue, and growing a transgenic plant from at least one of the identified transformants. In some embodiments, methods of making a transgenic plant include selecting transformants that produce fertile plants.

The polynucleotides and vectors described herein can be used to transform a number of monocotyledonous and dicotyledonous plants and plant cell systems, including dicots such as alfalfa, almond, amaranth, apple, apricot, avocado, beans (including kidney beans, lima beans, dry beans, green beans), brazil nut, broccoli, cabbage, canola, carrot, cashew, castor bean, cherry, chick peas, chicory, chocolate, clover, cocoa, coffee, cotton, cottonseed, crambe, eucalyptus, flax, foxglove, grape, grapefruit, hazelnut, hemp, jatropha, jojoba, lemon, lentils, lettuce, linseed, macadamia nut, mango, melon (e.g., watermelon, cantaloupe), mustard, neem, olive, orange, peach, peanut, peach, pear, peas, pecan, pepper, pistachio, plum, poplar, poppy, potato, pumpkin, oilseed rape, quinoa, rapeseed (high erucic acid and canola), safflower, sesame, soaptree bark, soybean, spinach, strawberry, sugar beet, sunflower, sweet potatoes, tea, tomato, walnut, and yams, as well as monocots such as banana, barley, bluegrass, coconut, corn, date palm, fescue, field corn, garlic, millet, oat, oil palm, onion, palm kernel oil, pineapple, popcorn, rice, rye, ryegrass, sorghum, sudangrass, sugarcane, sweet corn, switchgrass, turf grasses, timothy, and wheat. Gymnosperms such as fir, pine, and spruce can also be suitable.

Thus, the methods and compositions described herein can be used with dicotyledonous plants belonging, for example, to the orders Apiales, Arecales, Aristochiales, Asterales, Batales, Burseraceae, Campanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malpighiales, Malvales, Myricales, Myrtales, Nymphaeales, Papaverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ephedrales, Ginkgoales, Gnetales, and Pinales.

The methods and compositions can be used over a broad range of plant species, including species from the dicot genera Acokanthera, Aconitum, Aesculus, Alangium, Alchornea, Alexa, Alseodaphne, Amaranthus, Ammodendron, Anabasis, Anacardium, Angophora, Anisodus, Apium, Apocynum, Arabidopsis, Arachis, Argemone, Asclepias, Atropa, Azadirachta, Beilschmiedia, Berberis, Bertholletia, Beta, Betula, Bixa, Bleekeria, Borago, Brassica, Calendula, Camellia, Camptotheca, Canarium, Cannabis, Capsicum, Carthamus, Carya, Catharanthus, Centella, Cephaelis, Chelidonium, Chenopodium, Chrysanthemum, Cicer, Cichorium, Cinchona, Cinnamomum, Cissampelos, Citrus, Citrullus, Cocculus, Cocos, Coffea, Cola, Commiphora, Convolvulus, Coptis, Corylus, Corymbia, Crambe, Crotalaria, Croton, Cucumis, Cucurbita, Cuphea, Cytisus, Datura, Daucus, Dendromecon, Dianthus, Dichroa, Digitalis, Dioscorea, Duguetia, Erythroxylum, Eschscholzia, Eucalyptus, Euphorbia, Euphoria, Ficus, Fragaria, Galega, Gelsemium, Glaucium, Glycine, Glycyrrhiza, Gossypium, Helianthus, Heliotropium, Hemsleya, Hevea, Hydrastis, Hyoscyamus, Jatropha, Juglans, Lactuca, Landolphia, Lavandula, Lens, Linum, Litsea, Lobelia, Luffa, Lupinus, Lycopersicon, Macadamia, Mahonia, Majorana, Malus, Mangifera, Manihot, Meconopsis, Medicago, Menispermum, Mentha, Micropus, Nicotiana, Ocimum, Olea, Origanum, Papaver, Parthenium, Persea, Petunia, Phaseolus, Physostigma, Pilocarpus, Pistacia, Pisum, Populus, Prunus, Psychotria, Pyrus, Quillaja, Rabdosia, Raphanus, Rhizocarya, Ricinus, Rosa, Rosmarinus, Rubus, Rubia, Salix, Salvia, Sanguinaria, Scopolia, Senecio, Sesamum, Simmondsia, Sinapis, Sinomenium, Solanum, Sophora, Spinacia, Stephania, Strophanthus, Strychnos, Tagetes, Theobroma, Thymus, Trifolium, Trigonella, Vaccinium, Vicia, Vigna, Vinca, and Vitis; and the monocot genera Agrostis, Allium, Ananas, Andropogon, Areca, Asparagus, Avena, Cocos, Colchicum, Convallaria, Curcuma, Cynodon, Elaeis, Eragrostis, Festuca, Festulolium, Galanthus, Hemerocallis, Hordeum, Lemna, Lolium, Musa, Oryza, Panicum, Pennisetum, Phleum, Phoenix, Poa, Ruscus, Saccharum, Secale, Sorghum, Triticosecale, Triticum, Veratrum, Zea, and Zoysia; and the gymnosperm genera Abies, Cephalotaxus, Cunninghamia, Ephedra, Picea, Pinus, Populus, and Pseudotsuga.

A suitable group of species with which to practice the invention include alkaloid producing plants, e.g., plants from the Papaveraceae, Berberidaceae, Lauraceae, Menispermaceae, Euphorbiaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Rutaceae and Solanaceae families. The Papaveraceae family, for example, family contains about 250 species found mainly in the northern temperate regions of the world and includes plants such as California poppy and Opium poppy. Useful genera within the Papaveraceae family include the Papaver (e.g., Papaver bracteatum, Papaver orientale, Papaver setigerum, and Papaver somniferum), Sanguinaria, Dendromecon, Glaucium, Meconopsis, Chelidonium, Eschscholzioideae (e.g., Eschscholzia, Eschscholzia california), and Argemone (e.g., Argemone hispida, Argemone mexicana, and Argemone munita) genera. Other alkaloid producing species with which to practice this invention include Croton salutaris, Croton balsamifera, Sinomenium acutum, Stephania cepharantha, Stephania zippeliana, Litsea sebiferea, Alseodaphne perakensis, Cocculus laurifolius, Duguetia obovata, Rhizocarya racemifera, and Beilschmiedia oreophila.

Alkaloid producing species with which to practice this invention include Aconitum hemsleyanum, Aconitum spp., Alangium lamarkii, Alexa spp., Alseodaphne perakensis, Ammodendron spp., Anabasis sphylla, Anisodus tanguticus, Areca catechu, Argemone hispida, Argemone mexicana, Argemone munita, Argemone spp., Aspidospera subincanum, Atropa belladonna, Atropa spp., Beilschmiedia oreophila, Berberis spp., Bleekeria vitiensis, Camellia sinensis, Camptotheca acuminate, Castanosperma australe, Catharanthus roseus, Catharanthus spp., Cephaelis ipecacuanha, Cephalotaxus spp., Chondodendron tomentosum, Cinchona officinalis, Cinchona spp., Cissampelos pareira, Claviceps pupurea, Cocculus laurifolius, Coffea Arabica, Cola spp., Colchicum autumnale, Colchicum spp., Coptisjaponica, Crotalaria spp., Croton balsamifera, Croton salutaris, Cytisus scoparius, Datura sanguinera, Datura spp., Datura stomonium, Dichroafebrifuga, Duguetia obovata, Ecteinascidia turbinate, Ephedra sinica, Ephedra spp., Erythroxylum coca, Eschscholzia California, Eschsholtzia spp., Excavatia coccinea, Galanthus wornorii, Galega officinalis, Gelsemium sempervivens, Glauciumflavum, Glaucium spp., Heliotropium indicum, Hemsleya amabilis, Hydrastis Canadensis, Hyoscyamus spp., Litsea sebiferea, Lobelia spp., Lycopodium serratum (=Huperzia serrata), Lycopodium spp., Mahonia spp., Melodinus balansae, Merendera spp., Messerschmidia argentea, Nicotiana tabacum, Ochrosia spp., Papaver bracteatum, Papaver somniferum, Papaver spp., Pausinystalia yohimbe, Physostigma venenosum, Pilocarpus microphyllus, Pilocarpus spp., Pseudoxandra lucida, Psychotria spp., Rauwolfia canescens, Rauwolfia serpentina, Rauwolfia spp., Remijia pedunculata, Rhizocarya racemifera, Sanguinaria canadensis, Scopolia spp., Sinomenium acutum, Sophora pschycarpa, Sophora spp., Stephania cepharantha, Stephania sinica, Stephania tetrandra, Stephania zippeliana, Strychnos nux-vomica, Strychnos spp., Theobroma cacao, Tonduzia longifolia, Veratrum spp., Vinca minor, Vinca rosa, Vinca spp.

Another suitable group of species with which to practice the invention include terpenoid producing plants, e.g., plants from the genera Aesculus, Anamirta, Andrographis, Artemisia, Betula, Bixa, Cannabis, Centella, Chrysanthemum, Tanacetum, Cinnamomum, Citrullus, Luffa, Coleus, Curcuma, Cymbopogan, Daphne, Euphorbia, Glycine, Glycyrrhiza, Gossypium, Guayule, Hevea, Isodon, Rabdosia, Rabdosia, Mentha, Salvia, Rosmarinus, Simarouba, Taxus, Thymus, and Tripterygium.

In some embodiments, a terpenoid producing plant is a member of the species Artemisia annua, Ananus comosus, Andrographis paniculata, Bixa orellana, Brassica campestris, Brassica napus, Brassica oleracea, Calendula officinalis, Cannabis sativa, Chrysanthemum parthenium, Cinnamommum camphora, Coffea arabica, Coleus forskohlii, Digitalis lanata, Digitalis purpurea, Glycine max, Glycyrrhiza glabra, Lactuca sativa, Lycopersicon esculentum, Mentha piperita, Mentha spicata, Musa paradisiaca, Parthenium argentatum, Rosmarinus officinalis, Solanum tuberosum, Tanacetum parthenium, Taxus baccata, Taxus brevifolia, Theobroma cacao, Tripterygium wilfordii, Vitis vinifera, or Zea mays.

Another suitable group of species with which to practice the invention include phenylpropanoid producing plants, e.g., plants from the genera Camellia, Potentilla, Citrus, Lathyrus, Lonchocarpus, Silybum, Sophora, Glycine, Ammi, Heracleum, Curcuma, Curcuma, Cynara, Larrea, Podophyllum, Juniperus, Sassafras, Taiwania, Vitis, Aesculus, Atropa, Datura, Digitalis, Frazinus, Vaccinium, and Populus. Suitable species include Camellia sinensis, Potentilla fragarioides, Lonchocarpus nicou, Silybum marianum, Sophora japonica, Glycine, Ammi majus, Heracleum candicans, Curcuma zedoaria, Cynara scolymus, Larrea divaricata, Sassafras officinalis, Taiwania cryptomeroides, Vitis vinifera, Frazinus, and Populus trichocarpa.

A transformed cell, callus, tissue, or plant can be identified and isolated by selecting or screening the engineered plant material for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. After a polynucleotide is stably incorporated into a transgenic plant, it can be introduced into other plants using, for example, standard breeding techniques.

Transgenic plants can have an altered phenotype as compared to a corresponding control plant that either lacks the transgene or does not express the transgene. Phenotypic effects can be evaluated relative to a control plant that does not express the exogenous polynucleotide of interest, such as a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polynucleotide when the plant exhibits less than 10% (e.g., less than 9%, 8%, 7%, 6%, 5%, 4%, 3%, 2%, 1%, 0.5%, 0.1%, 0.01%, or 0.001%) of the amount of mRNA exhibited by the plant of interest. Expression can be evaluated using methods including, for example, RT-PCR, Northern blots, S1 RNAse protection, primer extensions and chip assays. It should be noted that if a polynucleotide is expressed under the control of a tissue-specific or broadly expressing promoter, expression can be evaluated in the entire plant or in a selected tissue. Similarly, if a polynucleotide is expressed at a particular time, e.g., at a particular time in development or upon induction, expression can be evaluated selectively at a desired time period.

VI. Two Component Systems

In some embodiments, compositions and methods described herein are based in part on the following components: 1) a transcription factor under the control of a selected plant promoter and 2) an upstream activation sequence (UAS) recognized by the transcription activator, operably linked to a nucleic acid that decreases expression of a methylation status polypeptide. These components, when combined genetically, result in a pattern of decreased transcription and expression of the methylation status polypeptide so that production of secondary metabolites can be controlled in a desired manner. See, U.S. Patent Publication 2005/0257293.

Components can be combined genetically by, inter alia, permitting seed development to occur on a plurality of first plants that have been pollinated by a plurality of second plants. The first plants have been transformed with a first exogenous nucleic acid construct. The first nucleic acid construct comprises a first transcription activator recognition site that is operably linked to a nucleic acid that decreases expression of a methylation status polypeptide. The first plants are male-sterile in some embodiments.

The second plants are male-fertile and have been transformed with an exogenous activator nucleic acid encoding a transcription activator and having a promoter operably linked thereto. The transcription activator is effective for binding to the first recognition site.

Upon pollination of the first plants by pollen from the second plants, seed development ensues. In some embodiments, the activator nucleic acid carried by the pollen is expressed prior to or during seed development, and the resulting transcription activator polypeptide activates transcription of the first nucleic acid in developing seeds on the female plants. Transcription of the first nucleic acid causes secondary metabolite production. Thus, secondary metabolite production is effectively controlled to occur in a desired seed tissue or at a desired stage of seed development.

In some embodiments, plants are grown from seeds derived by pollination of the first plants by pollen from the second plants. The activator nucleic acid carried by the pollen can be expressed during growth of such progeny plants, and the resulting transcription activator polypeptide activates transcription of the first nucleic acid in developing progeny plants. Transcription of the first nucleic acid causes secondary metabolite production. Thus, secondary metabolite production is effectively controlled to occur in vegetative tissue or during seed development on progeny plants.

In some embodiments a second nucleic acid is present in the first plants. The second nucleic acid comprises the coding sequence for an endogenous gene involved in secondary metabolite biosynthesis operably linked to a second transcription activator recognition site. Seeds in such embodiments contain the first and second nucleic acids and the transcription activator nucleic acid.

A transcription activator is a polypeptide that binds to a recognition site in DNA, resulting in an increase in the level of transcription from a promoter operably linked in cis with the recognition site. Suitable transcription activators include, without limitation, plant transcription activators, chimeric transcription activators and yeast transcription activators. Plant transcription activators typically are from a species that is in a different taxonomic genus from plants used in a method, are from a species that is geographically widely separated from plants used in a method, and/or are from a species where the timing or tissue specificity of naturally occurring expression differs from that occurring in plants used in a method. If desired, a transcription activator can be tested for its allergenic properties and those that are non-allergenic selected for use. Suitable transcription factors include YAP1, YAP2, SKO1, zinc finger protein MIG1, ABF1 and UME6, all of which are from yeast.

Many transcription activators have discrete DNA binding and transcription activation domains. Thus, DNA binding domain(s) and transcription activation domain(s) of a suitable transcription activator can be derived from different sources, i.e., can be a chimeric transcription activator. For example, a transcription activator can have a DNA binding domain derived from the yeast gal4 gene and a transcription activation domain derived from the VP16 gene of herpes simplex virus. In other embodiments, a transcription activator can have a DNA binding domain derived from a yeast HAP1 gene and the transcription activation domain derived from VP16. Populations of transgenic organisms or cells having a first nucleic acid construct comprising a nucleic acid that decreases expression of a methylation status polypeptide and an activator nucleic acid that encodes a transcription activator polypeptide can be produced by transformation, transfection, or genetic crossing. See, e.g., WO 97/31064 and WO 2006/009922.

In embodiments in which first plants contain two exogenous nucleic acids, a single activator nucleic acid can encode two different transcription activators, one of which binds to a first recognition site on the first nucleic acid and the other of which binds to a second recognition site on the second nucleic acid. Alternatively, two different transcription activators can be encoded by separate nucleic acids. In either case, each of the transcription activators can have a different expression pattern, e.g., the transcription activator for the first recognition site can be operably linked to a constitutive promoter and the transcription activator for the second recognition site can be operably linked to a maturing endosperm promoter. In other embodiments, both transcription activators are operably linked to different, maturing endosperm promoters.

A nucleic acid that decreases expression of a methylation status polypeptide is operably linked to a recognition site for the transcription activator that is used to activate transcription of the nucleic acid. For example, a gal4 UAS recognition site would be operably linked to such a nucleic acid when a chimeric gal4-VP16 chimeric transcription activator is to be used to activate transcription. As another example, a Hap1 recognition site is operably linked to such a nucleic acid when a chimeric Hap1-VP16 chimeric transcription activator is to be used to activate transcription. It will be appreciated that more than one copy of a UAS can be operably linked to a nucleic acid that decreases expression of a methylation status polypeptide, i.e., 2, 3, 4, 5, or more than 5 copies of a UAS can be used in order to achieve the desired level of activation of the nucleic acid. Typically, a basal promoter is also operably linked to the nucleic acid that decreases expression of a methylation status polypeptide.

A first plant containing a first nucleic acid as described herein and suitable for use in the invention can be identified by crossing with one or more second plants containing a transcription activator as described herein, followed by selecting or screening for modulated levels of one or more secondary metabolites in progeny. After a suitable first plant has been identified, the first nucleic acid can be introduced into other plants using, for example, standard breeding techniques.

VII. Articles of Manufacture

A plant seed composition can contain seeds of a first type of plant and of a second type of plant. Seeds of the first type of plant are of a single hybrid, inbred, line or variety, as are seeds of the second type of plant.

The proportion of seeds of each type of plant in a composition is measured as the number of seeds of a particular type divided by the total number of seeds in the composition, and can be formulated as desired to meet requirements based on geographic location, pollen quantity, pollen dispersal range, plant maturity, choice of herbicide, and the like. The proportion of the first type can be from about 70 percent to about 99.9 percent, e.g., 90%, 91%, 92%, 93%, 94%, 95%, 96%, 97%, 98%, or 99%. The proportion of the second type can be from about 0.1 percent to about 30 percent, e.g., 0.5%, 1%, 2%, 5%, 10%, 15%, 20%, 25%, or 30%. When large quantities of a seed composition are formulated, or when the same composition is formulated repeatedly, there may be some variation in the proportion of each type observed in a sample of the composition, due to sampling error. In the present invention, such sampling error typically is about ±5% of the expected proportion, e.g., 90%±4.5%, or 5%±0.25%.

For example, a seed composition can be made from two corn hybrids. A first corn hybrid can constitute 92% of the seeds in the composition and carries a first nucleic acid construct comprising a nucleic acid that decreases the amount of transcription or translation product of a gene encoding a methylation status polypeptide, operably linked to a recognition site for a transcription activator. The second corn hybrid can constitute 8% of the seed in the composition, is male-fertile, and carries an activation nucleic acid encoding a transcription activator that recognizes the transcription recognition site on the first nucleic acid construct. The transcription activator coding sequence is operably linked to a promoter that confers transcription in seeds. Such a seed composition can be used to grow plants that are suitable for practicing a method of the invention. See, e.g., PCT publications WO 2006/009922, WO 2004/027038 A3 and WO 03/025172.

Plants of the first type can be male-sterile, e.g., pollen is either not formed or is nonviable. Suitable male-sterility systems are known, including cytoplasmic male sterility (CMS), nuclear male sterility, genetic male sterility, and molecular male sterility wherein a transgene inhibits microsporogenesis and/or pollen formation. Female parent plants containing CMS are particularly useful. In the case of rice, see, e.g., U.S. Pat. No. 6,294,717. In the case of corn, a number of different methods of conferring male sterility are available, such as multiple mutant genes at separate locations within the genome that confer male sterility. In addition, one can use transgenes to silence one or more nucleic acid sequences necessary for male fertility. See, U.S. Pat. Nos. 4,654,465, 4,727,219, and 5,432,068. See also, EPO publication no. 329 308 and PCT application WO 90/08828.

Alternatively, plants of both the first type and the second type can be male-fertile. In this case, plants of the first type can be pollinated by hand, using pollen from plants of the second type. In some embodiments, pollen-forming structures on plants of the first type are removed in order to prevent self-pollination of first plants, thereby permitting manual or natural pollination by pollen from second plants. One can also use gametocides to inhibit or prevent pollen formation on plants of the first type. Gametocides are chemicals that affect cells critical to male fertility. Typically, a gametocide affects fertility only in the plants to which the gametocide is applied. Application of the gametocide, timing of the application and genotype can affect the usefulness of the approach. See, U.S. Pat. No. 4,936,904. In some embodiments, plants are of a species that exhibits partial or complete self-incompatibility. When complete self-incompatibility is present, measures such as male sterility systems or removal of pollen-forming structures on plants of the first type may not be necessary.

Typically, a substantially uniform mixture of seeds of each of the types is conditioned and bagged in packaging material by means known in the art to form an article of manufacture. Packaging material such as paper and cloth are well known in the art. Such a bag of seed preferably has a package label accompanying the bag, e.g., a tag or label secured to the packaging material, a label printed on the packaging material or a label inserted within the bag. The package label indicates that the seeds therein are a mixture of different plant types, e.g., two different varieties. The package label also may indicate the seed mixture contained therein incorporate transgenes that provide increased amounts of a secondary metabolite in tissues of such plants.

Plants grown from seeds in the composition typically have the same or very similar maturity, i.e., the same or very similar number of days from germination to crop seed maturation. In some embodiments, however, one or more of the seed types can have a different relative maturity compared to other varieties in the composition. The presence of plants of different relative maturities in a seed composition can be useful as desired to properly coordinate optimum pollen receptivity of the first type of plants with optimum pollen shed from the second type of plants. Relative maturity of a hybrid, inbred, line or variety of a given crop species is classified by techniques known in the art.

In some embodiments, a seed composition contains seeds of essentially a single plant type, e.g., a corn hybrid. The hybrid can be made by crossing two corn inbreds. The first corn inbred carries a first nucleic acid construct comprising a first nucleic acid that decreases the amount of transcription or translation product of a gene encoding a methylation status polypeptide, operably linked to an upstream activation region for a transcription activator. The second corn inbred carries an activator nucleic acid encoding a transcription activator that recognizes the upstream activation region on the first nucleic acid construct. The transcription activator coding sequence is operably linked to a broadly expressing promoter, e.g., p326 (SEQ ID NO: 76). The F₁ progeny of the cross can be placed in packaging material and used to grow plants that are suitable for practicing a method of the invention. See, e.g., PCT publications WO 2006/009922, WO 2004/027038 and WO 03/025172.

In some embodiments, plant cells are subjected to environmental conditions that facilitate the synthesis of increased amounts of a secondary metabolite. Environmental conditions under which a plant, or a plant or cell culture, is grown can be altered, e.g., by increasing the temperature, increasing the watering rate, or decreasing the watering rate, relative to a control temperature or watering rate. Other environmental conditions that can be altered in order to increase the amount or synthesis rate of a polypeptide include the concentration of salt, minerals, hormones, nitrogen, carbon, osmoticum, or known elicitors such as yeast extract, salicylic acid, and methyl jasmonate.

VIII. Secondary Metabolites Transgenic plants or cells grown according to the methods described herein produce one or more secondary metabolites, e.g., alkaloids, terpenoids, terpene indole alkaloids, flavonoids, and/or phenylpropanoids.

The amount of one or more of any individual secondary metabolite can be modulated, e.g., increased or decreased, relative to a control plant or cell lacking a nucleic acid that decreases expression of a gene encoding a methylation status polypeptide. In certain cases, more than one secondary metabolite, e.g., two, three, four, five, six, seven, eight, nine, ten, or even more secondary metabolites, can have its amount modulated relative to a control plant or cell. In some embodiments, at least one secondary metabolite will be detectable by the analytical technique used, whereas the secondary metabolite will not be detectable in a corresponding non-transgenic control using the same analytical technique. In some of these embodiments, the detectable secondary metabolite is a novel compound. In such instances, the novel compound may be new to the plant species or a new chemical entity.

Alkaloid Compounds

Alkaloid compounds are nitrogenous organic molecules that are typically derived from plants. Alkaloid biosynthetic pathways often include amino acids as reactants.

Alkaloid compounds can be mono-, bi-, or polycyclic compounds. Bi- or poly-cyclic compounds can include bridged structures or fused rings. Alkaloid producing plants or cells containing a recombinant nucleic acid construct described herein typically have a difference in the amount and/or rate of synthesis of one or more of alkaloid compounds, relative to a corresponding control plant or cell that is not transformed with the recombinant nucleic acid construct.

A number of different classes of alkaloid compounds, based on chemical and structural features, can be produced by the methods and compositions described herein. Such classes include, without limitation, tetrahydrobenzylisoquinoline alkaloids, morphinan alkaloids, benzophenanthridine alkaloids, monoterpenoid indole alkaloids, bisbenzylisoquinoline alkaloids, pyridine alkaloids, purine alkaloids, tropane alkaloids, quinoline alkaloids, terpenoid alkaloids, betaine alkaloids, steroid alkaloids, acridone alkaloids, and phenethylamine alkaloids. Other classifications may be known to those having ordinary skill in the art. Alkaloid compounds whose amounts are modulated relative to a control plant can be from the same alkaloid class or from different alkaloid classes.

In some embodiments, a morphinan alkaloid compound that is modulated is salutaridine, salutaridinol, salutaridinol acetate, isothebaine, thebaine, neopinone, codeinone, codeine, morphine, morphinone, papaverine, narcotine, narceine, hydrastine, or oripavine.

In other embodiments, a benzophenanthridine alkaloid compound is produced, e.g., dihydrosanguinarine, sanguinarine, dihydroxy-dihydrosanguinarine, 12-hydroxy-dihydrochelirubine, 10-hydroxy-dihydrosanguinarine, dihydromacarpine, dihydrochelirubine, dihydrosanguinarine, chelirubine, 12-hydroxychelirubine, or macarpine.

In still other embodiments, a tetrahydrobenzylisoquinoline alkaloid compound is produced, e.g., 2′-norberbamunine, S-coclaurine, S-norcoclaurine, R—N-methyl-coclaurine, S—N-methylcoclaurine, S-3′-hydroxy-N-methylcoclaurine, aromarine, S-3-hydroxycoclaurine, S-norreticuline, R-norreticuline, S-reticuline, R-reticuline, S-scoulerine, S-cheilanthifoline, S-stylopine, S-cis-N-methyl-stylopine, protopine, 6hydroxyprotopine, 1,2-dehydroreticuline, S-tetrahydrocolumbamine, columbamine, palmatine, tetrahydropalmatine, S-canadine, nororientaline, or berberine.

In some embodiments, a monoterpenoid indole alkaloid compounds is produced, e.g., vinblastine, vincristine, yohimbine, ajmalicine, ajmaline, and vincamine. In other cases, a pyridine alkaloid is produced. The pyridine alkaloid can be piperine, coniine, trigonelline, arecaidine, guvacine, pilocarpine, nicotine, and sparteine. A tropane alkaloid that can be produced includes atropine, cocaine, tropacocaine, hygrine, ecgonine, (−) hyoscyamine, (−) scopolamine, and pelletierine. A quinoline alkaloid that is produced can be quinine, strychnine, brucine, veratrine, or cevadine. Acronycine is an example of an acridone alkaloid.

In some cases, a phenylethylamine alkaloid is produced, e.g., MDMA, methamphetamine, mescaline, and ephedrine. In other cases, a purine alkaloid is produced, such as the xanthines, caffeine, theobromine, theacrine, and theophylline.

Bisbenzylisoquinoline alkaloids that can be produced include (+)-tubocurarine, dehatrine, (+)-thalicarpine, aromoline, guatteguamerine, berbamunine, and isotetradine. Yet other alkaloid compounds that can be produced include 3,4-dihydroxyphenylacetaldehyde.

Certain useful alkaloid compounds, with associated plant species that are capable of producing them, are listed in the Alkaloid Table, below.

Alkaloid Compound Table

| Alkaloid Name | Plant Source(s) | Alkaloid Name | Plant Source(s) |
|---|---|---|---|
| Oreobeiline | Beilschmiedia oreophila | Galanthamine | Galanthus wornorii |
| Hemsleyadine | Aconitum hemsleyanum, | Serpentine | Rauwolfvia spp. and |
| | Hemsleya amabilis | | Catharanthus spp. |
| Anabasine | Anabasis aphylla | Noscapine | Papaver somniferum |
| Aconitine | Aconitum spp. | Scopolamine | Atropa, Datura, Scopolia, |
| | | | Hyoscyamus spp. |
| Anisodamine | Anisodus tanguticus | Monocrotaline | Crotalaria spp. |
| Anisodine | Datura sanguinea | Apomorphine | Papaver somniferum |
| Arecoline | Areca catechu | Levallorphan | Papaver somniferum |
| Homatropine | Atropa belladonna | Hydrocodone | Papaver somniferum |
| Camptothecin | Camptotheca acuminata | Hydromorphone | Papaver somniferum |
| Orothecin | Camptotheca acuminata | Oxycodone | Papaver somniferum |
| 9-amino camptothecin | Camptotheca acuminata | Oxymorphone | Papaver somniferum |
| Topotecan | Camptotheca acuminata | Buprenorphine | Papaver somniferum |
| Irinotecan | Camptotheca acuminata | Nicotine | Nicotiana tabacum |
| Castanospermine | Castanospermum australe, Alexa | Sparteine | Cytisus scoparius, Sophora |
| | spp. | | pschycarpa, Ammodendron spp. |
| Vinorelbine | Catharanihus roseus | Oxandrin | Pseudoxandra lucida |
| Zippeline | Stephania zippeliana | Sarpagine | Rauwolfia & Vinca spp. |
| Homoharringtonine | Cephalotaxus spp. | Deserpidine | Rauwolfia canescens, |
| | | | Rauwolfia spp. |
| Harringtonine | Cephalotaxus spp. | Rescinnamine | Rauwolfia spp. |
| Quinidine | Cinchona spp., Remijia | Reserpine | Rauwolfia serpentina, |
| | pedunculata | | Rauwolfia spp. |
| Cissampareine | Cissampelos pareira | Matrine | Sophora spp. |
| Colchicine | Colchicum autumnale | Cabergoline | Claviceps pupurea |
| Demecolcine | Colchicum spp., Merendera spp. | Ellipticine | Ochrosia spp., Aspidospera |
| | | | subincanum, Bleekeria vitiensis |
| Glaucine | Glaucium flavum, Berberis spp. | 9-Methoxyellipticine | Ochrosia spp., Excavatia |
| | and Mahonia spp. | | coccinea, Bleekeria vitiensis |
| Physostigmine | Physostigma venenosum | Protoveratrines A, B | Veratrum spp. |
| Changrolin | Dichroa febrifuga | Cyclopamine | Veratrum spp. |
| a-Lobeline | Lobelia spp. | Veratramine | Veratrum spp. |
| Sinomenine | Sinomenium acutum and | Vasicine | Vinca minor, Galega officinalis |
| | Stephania cepharantha | | |
| Gelsemin | Gelsemium sempervivens | Vindesine | Vinca rosea |
| Hydrastine | Hydrastis canadensis | Vincamine | Vinca spp. |
| Indicine | Heliotropium indicum & | Cimetropium | Atropa, Datura, Scopolia, |
| | Messerschmidia argentea | Bromide | Hyoscyamus spp. |
| Rotundine | Eschsholtzia californica, | Huperzine A | Lycopodium serratum (=Huperzia |
| | Stephania sinica, Eschsholtzia | | serrata), Lycopodium |
| | spp., Argemone spp. | | spp. |
| Hyoscyamine | Hyoscyamus, Atropa, Datura, | Ecteinascidin 743 | Marine tunicate - Ecteinascidia |
| | Scopolia spp. | | turbinata |
| Flavinine | Litsea sebiferea, Alseodaphne | Emetine | Alangium lamarckii, Cephaelis |
| | perakensis, Cocculus laurifolius, | | ipecacuanha, Psychotria spp. |
| | Duguetia obovata and Rhizocarya | | |
| | racemifera | | |
| Tetrandrine | Stephania tetrandra | | |

Terpenoid Compounds

A number of different classes of terpenoid compounds, based on chemical and structural features, can be produced by the methods and compositions described herein. Such classes include, without limitation, monoterpenoids, monoterpenoid lactones, sesquiterpenoids, sesquiterpenoid lactones, diterpenoids, triterpenoids, carotenoids, steroids, and sterols. Plants containing a recombinant nucleic acid construct described herein typically have a difference in the amount and/or rate of synthesis of one or more of terpenoid compounds, relative to a corresponding control plant or cell that is not transformed with the recombinant nucleic acid construct.

In some embodiments, a monoterpenoid compound is produced, e.g., geranyl diphosphate, linalyl acetate, carvone, nerol, menthol, β-ocimene, pinene, limonene, 1,8 cineole, myrcene, (+)-bornyl diphosphate, (−)-isopiperitenone, (+)-pulegone, (−)-menthone, thujone, marinol, tetrahydrocannabinol, camphor, borneol, perillyl alcohol, thymol, sobrerol, or sabinene.

In other embodiments, a sesquiterpene or sesquiterpene-derived compound is produced, such as farnesyl diphosphate, E-β-farnesene, β-caryophyllene, 5-epi-aristolochene, vetispiradiene, δ-cadinene, germacrene C, E-α-bisabolene, δ-selinene, parthenolide, artemisinin, artemisin, artemether, santonin, parthenolide, gossypol, manoalide, acetyldigoxin, digoxin, deslanoside, digitalin, digitoxin, lanatosides A, B and C or γ-humulene.

In yet other embodiments, a diterpene or diterpene-derived compound is produced such as geranylgeranyl diphosphate, ent-copalyl diphosphate, ent-kaurene, taxadiene, taxol, baccatin III, calanolide A, ginkgolides, casbene, abietadiene, andrographolide, neoandrographolide, forskolin, resiniferatoxin, pseudopterosin C, methopterosin, carnosic acid, camosol, tanshinone II-A, saprorthoquinone, triptolide or cambrene.

In some embodiments, a triterpenoid or steroid is produced, such as squalene, lupeol, α-amyrin, β-amyrin, glycyrrhizin, β-sitosterol, sitostanol, stigmasterol, α-tocopherol, β-tocopherol, γ-tocopherol, campesterol, ergosterol, diosgenin, aescin, picrotoxin, betulinic acid, asiaticoside, cucurbitacin E, glycyrrhizin, diosgenin or ruscogenin.

In yet other embodiments, a tetra- or polyterpene is produced, such as lycopene, β-carotene, zeta-carotene, lutein, zeaxanthin, and antheraxanthin, phytoene, bixin and astaxanthin. Other terpenoid compounds that can be produced and/or extracted by methods described herein include yuanhuacin, yuanhuadin, glaucarubin, convallatoxin, squalamine, ouabain, or strophanthidin.

Phenylpropanoid Compounds

A number of different classes of phenylpropanoid compounds, based on chemical and structural features, can be produced by the methods and compositions described herein. Such classes include, without limitation, flavonoids, catechins, isoflavones, anthocyanins, stilbenes, chalcones, lignans, coumarins, and lignin. Exemplary phenylpropanoid compounds that can be produced and/or extracted by methods described herein include (+)-catechin, hesperidin, rutin, malvidin, rotenone, silymarin (silybin), troxerutin, quercetin, flavopiridol, genistein, daidzein, methoxsalen, curcumin, cynarin, nordihydroguaiaretic acid (NDGA), masoprocol, podophyllotoxin, etoposide, teniposide, saffrole, gomisin A, taiwanin A, psoralen, resveratrol, oxypsoralen, rosmarinic acid, warfarin, aesculetin, cyanidin and lignin.

Increases in Secondary Metabolite Amounts

The amount of one or more secondary metabolites can be increased or decreased in transgenic cells comprising a recombinant nucleic acid construct as described herein. The amount of a secondary metabolite that is produced can be determined by known techniques, e.g., by extraction of secondary metabolites followed by gas chromatography-mass spectrometry (GC-MS) or liquid chromatography-mass spectrometry (LC-MS). If desired, the structure of a particular compound(s) can be confirmed by GC-MS, LC-MS, nuclear magnetic resonance and/or other known techniques.

An increase in the amount of a secondary metabolite can be from about 5% to about 500% on a weight basis (e.g., a fresh weight or dry weight basis) in such a transgenic cell compared to a corresponding control cell that lacks the recombinant nucleic acid. In some embodiments, the increase is from about 5% to about 250%, or about 50% to about 500%, or about 100% to about 400%, or about 25% to about 400%, or about 50% to about 350%, or about 75% to about 150%, or about 90% to about 250%, or about 125% to about 375%, or about 150% to about 450% higher than the amount in a corresponding control cell that lacks the recombinant nucleic acid. In some embodiments, the increase is from about 1.5-fold to about 450-fold, e.g., about 2-fold to about 22-fold, or about 25-fold to about 50-fold, or about 75-fold to about 130-fold, or about 5-fold to about 50-fold, or about 5-fold to about 10-fold, or about 10-fold to about 20-fold, or about 10-fold to about 25-fold, or about 20-fold to about 75-fold, or about 10-fold to about 100-fold, or about 40-fold to about 100-fold, or about 30-fold to about 50-fold higher than the amount in a corresponding control cell that lacks the recombinant nucleic acid construct.

In other embodiments, a secondary metabolite is produced in transgenic plants or cells in which the secondary metabolite is either not produced or is not detectable in a corresponding control plant or cell. Thus, in such embodiments, the increase in such a secondary metabolite is infinitely higher than in a corresponding control cell. That is, the decrease in expression of a methylation status polypeptide is contemplated to activate a biosynthetic pathway in the plant that is not normally activated or operational, thereby producing one or more new compounds that are not normally produced in that plant species.

The increase in amount of one or more secondary metabolites can be restricted in some embodiments to particular tissues and/or organs, relative to other tissues and/or organs. For example, a transgenic plant can have an increased amount of an alkaloid in inflorescence tissue relative to leaf tissue.

When expression of a methylation status polypeptide is decreased in specific cells or tissues, secondary metabolites can be produced in those same cells or tissues. In other embodiments, secondary metabolites are produced in cells or tissues other than the cells in which expression of a methylation status polypeptide is decreased. For example, expression of a methylation status polypeptide can be decreased preferentially in a first cell type or tissue, e.g., a gametophytic tissue, which leads to production of sterol compounds in a second cell type or tissue, e.g., endosperm or embryo tissue. As another example, a first transgenic plant tissue or organ culture can be grown, such as a feeder layer, in the presence of a second plant tissue or organ culture. Sterol compounds from the first tissue or organ culture can diffuse in media, be taken up by cells of the second tissue or organ culture and may lead to production of other sterol compounds in the second tissue. It will be appreciated that the first tissue or organ may or may not express the down regulator of a methylation status polypeptide.

In some embodiments, plant cells are subjected to environmental conditions that facilitate the synthesis of increased amounts of secondary metabolites. Environmental conditions under which a plant, or a plant or cell culture, is grown can be altered, e.g., by increasing the temperature, increasing the watering rate, or decreasing the watering rate, relative to a control temperature or watering rate. Other environmental conditions that can be altered in order to increase the amount or synthesis rate of secondary metabolites include the concentration of salt, minerals, hormones, nitrogen, carbon, osmoticum, or known elicitors such as yeast extract, salicylic acid, and methyl jasmonate.

Typically, an increase in the amount of a secondary metabolite in cells of a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of a secondary metabolite is statistically significant at p<0.01, p<0.005, or p<0.001. A statistically significant difference in, for example, the amount of a secondary metabolite in cells of a transgenic plant compared to the amount in cells of a control plant indicates that (1) the recombinant nucleic acid present in the transgenic plant results in an altered amount of a secondary metabolite in cells and/or (2) the recombinant nucleic acid warrants further study as a candidate for altering the amount of a secondary metabolite in a plant.

Decreases in Secondary Metabolite Amounts

In other embodiments, the amounts of one or more secondary metabolites are decreased in transgenic cells comprising a recombinant nucleic acid construct as described herein. A decreased ratio can be expressed as the ratio of the amount of a compound in such a transgenic cell on a weight basis (e.g., fresh or freeze dried weight basis) as compared to the amount in a corresponding control cell that lacks the recombinant nucleic acid construct. The decrease ratio can be from about 0.05 to about 0.90. In certain case, the ratio can be from about 0.2 to about 0.6, or from about 0.4 to about 0.6, or from about 0.3 to about 0.5, or from about 0.2 to about 0.4. A decrease in the amount of a secondary metabolite in cells of a transgenic plant or cell relative to a control plant or cell is considered statistically significant at p≦0.05 with an appropriate parametric or non-parametric statistic, as discussed above.

The decrease in amount of one or more secondary metabolites can be restricted in some embodiments to particular tissues and/or organs, relative to other tissues and/or organs. For example, a transgenic plant can have a decreased amount of an alkaloid in inflorescence tissue relative to leaf tissue.

Extraction of Secondary Metabolites

The invention also features methods in which one or more secondary metabolites are extracted from plant cells containing a recombinant nucleic acid construct described herein. Suitable tissues or organs from which secondary metabolites can be extracted include leaves, roots, stems, cambial cells, flowers, seeds, immature flower pods, seed capsules, embryos, endosperm, cotyledons, trichomes, meristematic tissue, embryogenic cultures, organogenic cultures, or liquid suspension cultures.

In some instances, plant cells in which a secondary metabolite is known or suspected of being present can be separated from cells in which the secondary metabolite is not suspected of being present. Such a separation can enrich for cells or cell types that contain such compounds. A number of methods for separating particular cell types or cell layers are known to those having ordinary skill in the art. For example, cell types may be dissected using laser capture microdissection, or can be captured using a cell sorter by virtue of an epitope tag in a reporter or receptor.

Typically, fractionation of extracts of plant cells in which a secondary metabolite is known or suspected of having been modulated is guided by information regarding the solubility of the known or suspected compound(s). Fractionation can be carried out by techniques known in the art. For example, plant tissue can be extracted with 100% MeOH to give a crude oil which is partitioned between several solvents in a conventional manner. As an alternative, fractionation can be carried out on gel columns using methylene chloride and ethyl acetate/hexane solvents.

In other embodiments, a fractionated or unfractionated plant tissue or organ extract is subjected to mass spectrometry in order to identify and characterize one or more secondary metabolites. See, e.g., WO 02/37111. In some embodiments, electrospray ionization (ESI) mass spectrometry can be used. In other embodiments, atomospheric pressure chemical ionization (APCI) mass spectrometry is used. If it is desired to identify higher molecular weight molecules in an extract, matrix-assisted laser desorption/ionization time-of-flight (MALDI-TOF) mass spectrometry can be useful.

Methods described herein permit extraction of one or more secondary metabolites from a wide-ranging group of plants and, if desired, permit extraction to be focused on particular tissues or organs in such plants. Such extracts can be crude extracts, partially purified extracts, or extensively purified extracts. Such extracts can be aqueous extracts or non-aqueous extracts.

Genes Involved in Secondary Metabolite Biosynthesis

Methods described herein can involve production of one or more secondary metabolites from a plant or cell comprising a second exogenous nucleic acid construct effective for modulating the amount of one or more endogenous genes that are involved in secondary metabolite biosynthesis in the plant. In some embodiments, a method comprises extracting one or more secondary metabolites from such plants or cells. The one or more secondary metabolites can be extracted as described above.

Endogenous genes involved in secondary metabolite biosynthesis typically are native, i.e., are unmodified by recombinant DNA technology from the sequence and structural relationships that occur in nature. The coding sequence of an endogenous gene typically encodes a polypeptide involved in secondary metabolite biosynthesis, e.g., an enzyme involved in biosynthesis of the secondary metabolite compounds described herein. An endogenous protein can also be a regulatory or auxiliary protein involved in transcription, e.g., transcription of terpenoid biosynthesis genes. Other components that may be present in an endogenous gene include promoters, introns, enhancers, upstream activation regions, and inducible elements. In some embodiments, however, the coding sequence from an endogenous gene can be operably linked to non-native regulatory regions, e.g., promoters as described herein (SEQ ID NOS: 1-103).

Alkaloid Biosynthesis Genes

In some instances, an endogenous gene can encode an enzyme involved in alkaloid biosynthesis. Such enzymes include those involved in morphinan alkaloid biosynthesis, e.g., an enzyme selected from the group consisting of salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); codeinone reductase (CR; EC 1.1.1.247); and other enzymes related to the biosynthesis of morphinan/opiate alkaloids.

In some instances, an endogenous gene can encode an enzyme involved in tetrahydrobenzylisoquinoline alkaloid biosynthesis, e.g., an enzyme selected from the group consisting of those encoding for tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC 1.14.18.1), N-methylcoclaurine 3′-hydroxylase (NMCH; EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; EC 1.21.3.3), reticuline oxidase (EC 1.21.3.4), dehydroreticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), columbamine oxidase (EC 1.21.3.2), and other enzymes related to the biosynthesis of tetrahydrobenzylisoquinoline alkaloids.

In some instances, an endogenous gene can encode an enzyme involved in benzophenanthridine alkaloid biosynthesis, e.g., an enzyme selected from the group consisting of those encoding for dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), 12-hydroxy-dihydrochelirubine 12-O-methyltransferase (EC 2.1.1.120), dihydrobenzophenanthridine oxidase, dihydrosanguinarine 10-monooxygenase, protopine 6-monooxygenase, and other enzymes related to the biosynthesis of benzophenanthridine alkaloids.

In some instances, an endogenous gene can encode an enzyme involved in purine alkaloid (e.g., xanthines, such as caffeine) biosynthesis such as xanthosine methyltransferase, 7-N-methylxanthine methyltransferase (theobromine synthase), or 3,7-dimethylxanthine methyltransferase (caffeine synthase).

In some instances, an endogenous gene can encode an enzyme involved in biosynthesis of indole alkaloids compounds such as tryptophane decarboxylase, strictosidine synthase, strictosidine glycosidase, dehydrogeissosshizine oxidoreductase, polyneuridine aldehyde esterase, sarpagine bridge enzyme, vinorine reductase, vinorine synthase, vinorine hydroxylase, 17-O-acetylajmalan acetylesterase, or norajamaline N-methyl transferase. In other embodiments, a suitable endogenous gene encodes an enzyme involved in biosynthesis of vinblastine, vincristine and compounds derived from them, such as tabersonine 16-hydroxylase, 16-hydroxytabersonine 16-O-methyl transferase, desacetoxyvindoline 4-hydroxylase, or desacetylvindoline O-acetyltransferasesynthase.

In still other instances, an endogenous gene can encode an enzyme involved in biosynthesis of pyridine, tropane, and/or pyrrolizidine alkaloids such as arginine decarboxylase, spermidine synthase, ornithine decarboxylase, putrescine N-methyl transferase, tropinone reductase, hyoscyamine 6-beta-hydroxylase, diamine oxidase, and tropinone dehydrogenase.

Terpenoid Biosynthesis Genes

In some instances, an endogenous gene can encode an enzyme involved in terpenoid biosynthesis. Terpenoid biosynthesis enzymes include those involved in isoprenoid biosynthesis via the mevalonic acid pathway, such as acetyl CoA acetyl (ACA) transferase (EC 2.3.1.9), hydroxy methyl glutaryl-CoA (HMG-CoA) synthase (EC 4.1.3.5), hydroxy methyl glutaryl-CoA (HMG-CoA) reductase (EC 1.1.1.34), mevalonate kinase (EC 2.7.1.36), mevalonate phosphate kinase (EC 2.7.4.2), mevalonate pyrophosphate decarboxylase (EC 4.1.1.33), or isopentenyl pyrophosphate (IPP) isomerase (EC 5.3.3.2). In other embodiments, a suitable endogenous gene encodes an enzyme involved in isoprenoid biosynthesis via the deoxyxylulose phosphate pathway such as deoxyxylulose phosphate synthase deoxyxylulose phosphate reductoisomerase, diphosphocytidyl methylerythritol transferase, diphosphocytidyl methylerythritol kinase, methylerythritol phosphocytidine diphosphate synthase, hydroxymethyl butiryl diphosphate synthase, or isopentenyl diphosphate synthase.

In some embodiments, a suitable endogenous gene encodes an enzyme involved in biosynthesis of monoterpenes and monoterpene-derived compounds such as geranyl diphosphate synthase (EC 2.5.1.1), β-ocimene synthase, pinene synthase (EC 4.2.3.14), limonene synthase (EC 4.2.3.16), 1,8 cineole synthase, myrcene synthase (EC 4.2.3.15), bornyl diphosphate synthase, (−)-isopiperitenone reductase (EC 5.3.3.11), (+)-pulegone reductase, (−)-menthone reductase, or sabinene synthase. In other embodiments, a suitable endogenous gene encodes an enzyme involved in biosynthesis of sesquiterpenes and sesquiterpene-derived compounds such as farnesyl diphosphate synthase (EC 2.5.1.10), E-β-farnesene synthase, β-caryophyllene synthase, 5-epi-aristolochene synthase (EC 4.2.3.9), vetispiradiene synthase (EC 4.2.3.21), δ-cadinene synthase (EC 4.2.3.13), germacrene C synthase, E-α-bisabolene synthase, δ-selinene synthase, and γ-humulene synthase. In yet other embodiments, a suitable endogenous gene encodes an enzyme involved in biosynthesis of diterpenes and diterpene-derived compounds such as geranylgeranyl diphosphate synthase, ent-copalyl diphosphate synthase (EC 5.5.1.12), ent-kaurene synthase (EC 1.14.13.78), taxadiene synthase (EC 4.2.3.17), casbene and cambrene synthase (EC 4.2.3.8), 3′-N-debenzoyl-2′-deoxytaxol N-benzoyltransferase, taxoid 2α-hydroxylase, taxoid 7β-hydroxylase, taxane 13α-hydroxylase (EC 1.14.13.77), taxane 10β-hydroxylase (EC 1.14.13.76), taxadiene 5α-hydroxylase (EC 1.14.99.37), taxadien-5α-ol-O-acetyltransferase, 10-deacetylbaccatin III-10β-O-acetyltransferase (EC 2.3.1.167), taxane 2α-O-benzoyltransferase, and abietadiene synthase (EC 4.2.3.18). In some embodiments, a suitable endogenous gene encodes an enzyme involved in triterpene biosynthesis such as squalene synthase, lupeol synthase, Arabidopsis pentacyclic synthase, and α and β-amyrin synthases. In yet other embodiments, a suitable endogenous gene encodes an enzyme involved in tetra- and polyterpene biosynthesis such as phytoene synthase (EC 2.5.1.32), phytoene desaturase, lycopene β-cyclase, lycopene ε-cyclase, β-carotene hydroxylase, z-carotene desaturase, zeaxanthin/antheraxanthin de-epoxidase, and zeaxanthin/antheraxanthin epoxidase. In some embodiments, a suitable endogenous gene encodes an enzyme involved in sterol synthesis, such as sterol methyl oxidase, C-8,7 sterol isomerase, and sterol methyl transferase2, which are involved in β-sitosterol synthesis.

In some embodiments, a suitable endogenous gene encodes an enzyme involved in artemisinin synthesis, such as amorpha-4,11-diene synthase. In yet other embodiments, a suitable endogenous gene encodes an enzyme involved in tetrahydrocannabinol synthesis, such as delta(1)-tetrahydrocannabinolic acid (THCA) synthase or geranyl diphosphate:olivetolate geranyltransferase (GOT). In yet other embodiments, a suitable endogenous gene encodes an enzyme involved in pseudopterosin C synthesis, such as elisabethatriene synthase. In yet other embodiments, a suitable endogenous gene encodes an enzyme involved in sterol synthesis, such as sterol methyl oxidase, C-8,7 sterol isomerase, and sterol methyl transferase2, which are involved in β-sitosterol synthesis. In yet other embodiments, a suitable endogenous gene encodes an enzyme involved in Vinca alkaloid synthesis, such as geraniol 10-hydroxylase, deoxyloganin 7-hydroxylase, and secologanin synthase.

Phenylpropanoid Biosynthesis Genes

In some instances, an endogenous gene can encode an enzyme involved in phenylpropanoid biosynthesis. Such enzymes include, without limitation, cinnamic acid 4-hydroxylase (EC 1.14.13.11), 4-coumaroyl-CoA synthetase (EC 6.2.1.12), chalcone synthase (EC 2.3.1.74), chalcone isomerase (EC 5.5.1.6), flavonoid 3′-hydroxylase (EC 1.14.13.21), flavanone 3-hydroxylase (EC 1.14.11.9), flavonol 3-O-glucosyltransferase (EC 2.4.1.91), flavonol-3-O-glucoside L-rhamnosyltransferase (EC 2.4.1.159), isoflavone synthase (EC 1.14.14.-), beta-peltatin 6-O-methyltransferase, secoisolariciresinol dehydrogenase, pinoresinol/lariciresinol reductase, resveratrol synthase (EC 2.3.1.95), dihydroflavanol 4-reductase (EC 1.1.1.219), cinnamyl-alcohol dehydrogenase (EC 1.1.1.195), pyrocatechol peroxidase (EC 1.11.1.7) and cinnamoyl-CoA reductase (EC 1.2.1.44).

The ability to produce one or more secondary metabolites in plants can provide advantages to agricultural producers and to consumers. For example, an increase in the amount of an alkaloid compound can provide increased yield of the compound when extracted from plant tissue, thereby providing an economic benefit to farmers. As another example, an increase in the amount of a terpenoid compound can provide improved nutritional and/or taste qualities to fruits and vegetables. As another example, an increase in the amount of one or more sterol compounds can provide increased yield of the compound when extracted from plant tissue, thereby providing a more economically efficient production system. As another example, an increase in the amount of a sterol can provide improved nutritional and/or taste qualities to grains. As another example, the ability to modulate secondary metabolite levels can lead to the production of new chemical entities with useful pharmaceutical properties, thereby providing a contribution to human and animal health care.

A number of embodiments of the invention have been described. Nevertheless, it will be understood that various modifications may be made without departing from the spirit and scope of the invention. The invention will be further described in the following examples, which do not limit the scope of the invention described in the claims.

### EXAMPLES

### Example 1

### Rice Methyltransferase RNAi Construct (35S::OsMET1Ct-RNAi)

An RNAi construct was made by operably linking a CaMV35S promoter to a sequence effective for being transcribed into an interfering RNA. The RNAi sequence comprised about 600 nucleotides of a rice cytosine DNA methyltransferase sense strand (N-terminal region) and an inverted repeat of a nos terminator sequence. The construct was made using standard molecular biology techniques. The sequence of the 35S::rice MET::inverted nos construct is shown in SEQ ID NO: 104. The rice MET portion of the construct is shown in SEQ ID NO: 105. The construct was inserted into a vector that contained a selectable marker gene conferring resistance to the herbicide Basta®.

A second RNAi construct was made by operably linking a CaMV35S promoter to a sequence effective for being transcribed into an interfering RNA. The RNAi sequence comprised about 660 nucleotides of a rice cytosine DNA methyltransferase sense strand (C-terminal region) and an inverted repeat of a nos terminator sequence. The construct was made using standard molecular biology techniques. The sequence of the 35S::rice MET::inverted nos construct is shown in SEQ ID NO: 106. The rice MET portion of the construct is shown in SEQ ID NO: 107. The construct was inserted into a vector that contained a selectable marker gene conferring resistance to the herbicide Basta®.

### Example 2

### Chemical Analysis of Terpenoid Compounds in Transgenic Rice

The following symbols are used in this Example: T1: plant regenerated from transformed tissue culture; T2: first generation progeny of self-pollinated T1 plants; T3: second generation progeny of self-pollinated T2 plants; T4: third generation progeny of self-pollinated T3 plants.

The second RNAi construct vector of Example 1 (SEQ ID NO: 106) was introduced into a tissue culture of the rice cultivar Kitaake by an Agrobacteriummediated transformation protocol. Approximately 20 independent T1 transgenic plants were generated from the transformation by selecting for Basta® resistance, as well as for the control plasmid (empty vector NB42-35S—RNAi). Preliminary phenotypic analysis indicated that T₁ transformants did not show any significant phenotypic anomalies in vegetative organs, with a few exceptions where some plants appeared to be shorter than the rest of the T1 plants. However, any variation in plant height may be due to tissue culture stress.

T1 plants were allowed to self-pollinate, T2 seeds were germinated and plants grown in a greenhouse. The presence of the RNAi construct was confirmed by PCR. Most of the T1 plants displayed severe fertility defects. In some case, inflorescences developed to maturity, but pistil development arrested soon after pollination. In other cases, plants had no inflorescence. These defects were observed in the majority of T1 plants. Occasionally, it was possible to obtain a few fully developed seeds from each inflorescence. A summary of seed phenotypes observed in individual T1 plants is presented in the Table below.

TABLE 1

| Transformant | Seed Phenotype |
|---|---|
| CT-1A | very few seeds |
| CT-2A | very few seeds |
| CT-3A | no seeds² |
| CT-4A | very few seeds |
| CT-5A | no seeds |
| CT-6A | no seeds |
| CT-7A¹ | no inflorescence |
| CT-8A | no seeds |
| CT-9A | few seeds |
| CT-10A | no seeds |
| CT-11A | no seeds |
| CT-12A | no seeds |
| CT-13A | no seeds |
| CT-14A | no seeds |
| CT-15A | normal seeds |
| CT-16A | few seeds |
| CT-17A | no seeds |
| CT-18A | no seeds |
| CT-20A | few seeds |
| CT-2B | very few seeds |
| CT-9B | no seeds |
| CT-13B | no seeds |

¹= small leaves
²= “no seeds” indicates that an inflorescence was present, but no seeds formed

Leaf tissue from four T₁ generation plants from each of four events, CT1A, CT10A, CT12A and CT17A were collected at mature stages. Leaf tissue from each event was pooled and analyzed for the amount of campesterol, stigmasterol, sitosterol and alpha-tocopherol. As a control, leaf tissue from transgenic control Kitaake T₁ plants was analyzed at the same stage in development. The results showed that the amounts of campesterol, stigmasterol, sitosterol and α-tocopherol in T₁ leaf tissue were not statistically different from the corresponding amounts in the control tissue for any of the four events.

About three inflorescences from four T₁ generation plants from each of the four events were collected before pollination. Inflorescences from each event were pooled and analyzed in triplicate for the amount of campesterol, stigmasterol, β-sitosterol and alpha-tocopherol. As a control, inflorescences from transgenic control Kitaake T₁ plants were analyzed at the same stage in development. Transgenic control plants contained the control vector described above. The results, shown in Table 2, indicated that the amount of campesterol was significantly higher in inflorescences produced by T₁ plants from events CT10 and CT12 compared to the corresponding amount in control inflorescences. The amounts of stigmasterol and β-sitosterol were significantly higher in inflorescences produced by T₁ plants from events CT10, CT12 and CT17 compared to the corresponding amounts in controls. The amount of α-tocopherol was significantly higher in inflorescences produced by T₁ plants from event CT12 compared to the corresponding amount in controls. The amounts of α-tocopherol were significantly decreased in events CT1, CT10 and CT17 relative to the controls.

TABLE 2

| | Terpenoid accumulation in | | | | |
|---|---|---|---|---|---|
| | transgenic OsMET1-RNAi rice plants | | | | |
| Compound* | Control (%) | CT1 (%) | CT10 (%) | CT12 (%) | CT17 (%) |
| Campesterol | 100 ± 1.73 | 101.58 ± 2.39 | 140.64 ± 2.44 | 150.15 ± 1.42 | 101.85 ± 1.80 |
| Stigmasterol | 100 ± 1.46 | 154.56 ± 7.01 | 159.29 ± 7.90 | 165.58 ± 9.65 | 113.69 ± 4.59 |
| β-Sitosterol | 100 ± 3.76 | 162.64 ± 7.69 | 168.93 ± 15.11 | 135.80 ± 3.68 | 117.81 ± 9.15 |
| α-Tocopherol | 100 ± 3.85 | 46.05 ± 15.60 | 88.16 ± 3.60 | 313.06 ± 4.42 | 75.39 ± 2.07 |

*The amount of each compound is expressed as percent of control.

### Example 3

### OsMET1-1 and OsMET1-2 Transcription in Inflorescence Tissues

Previously, it has been reported that expression of OsMET1-1 and OsMET1-2 is active in callus, root and inflorescence, and that the steady-state level of OsMET1-2 is 7to 12-fold higher than that for OsMET1-1 in these tissues. Teerawanichpan P, et al. Planta 218:337-49 (2004). In addition, it was reported that no transcript of OsMET1-2 was detectable in differentiated tissue (10-day-old leaf), and no expression for either gene was found in mature leaves.

Total RNA was isolated from whole-inflorescence tissues from ten of the T₁ plants described in Example 2, and qRT-PCR analysis was performed using gene-specific primers for OsMET1-1 and OsMET1-2. Inflorescence tissue collected from plants transformed with an empty vector was used as the control. The results are shown in Table 3. The majority of T₁ CT and NT transgenic plants had reduced transcription of OsMET1 genes. For example, endogenous OsMET1-1 transcript levels in plant CT1 inflorescences were diminished by up to 95%. Endogenous OsMET1-2 transcript levels in plant CT1 inflorescences were reduced by up to 99%.

TABLE 3

| | OsMET1-1 | OsMET1-2 |
|---|---|---|
| Transformant | (%)* | (%)* |
| CT-1A | 6 | 1 |
| CT-2A | 83 | 56 |
| CT-3A | 47 | 34 |
| CT-4A | 138 | 16 |
| CT-5A | 56 | 95 |
| CT-6A | 36 | 48 |
| CT-7A | — | — |
| CT-8A | — | — |
| CT-9A | 54 | 36 |
| CT-10A | 32 | 58 |
| CT-11A | — | — |
| CT-12A | 48 | 25 |
| CT-13A | — | — |
| CT-14A | — | — |
| CT-15A | — | — |
| CT-16A | — | — |
| CT-17A | 22 | 28 |
| CT-18A | — | — |
| CT-20A | — | — |
| CT-2B | — | — |
| CT-9B | — | — |
| CT-13B | — | — |

*Amount of transcript in inflorescences relative expressed as a percentage of amount in controls
—: Not done

### Example 4

### Preparation of Transgenic Rice Containing Different Rice Methyltransferase RNAi Constructs

Rice cells were transformed as described in Example 2 with three different MET 1 RNAi constructs. Each construct was the same as the second construct described in Example 1, except that the 35S promoter was replaced with one of the following rice tissue-specific regulatory regions: pOsFIE2-2, pOsMEA and p530c10. See, SEQ ID NOS: 90, 91 and 97, respectively.

T₁ plants from independent transformation events containing the p530c10, pOsFIE2-2 and pOsMEA constructs are regenerated from tissue selected for Basta® resistance, and are allowed to self-pollinate. Inflorescences from transgenic T₁ plants are analyzed for the amounts of campesterol, stigmasterol, β-sitosterol and α-tocopherol.

### Example 5

### Preparation of Transgenic Corn Containing Two Component Constructs

Corn cells are transformed with a MET 1 RNAi construct. The construct is the same as the second construct described in Example 1, except that the 35S promoter is replaced with 4 copies of an upstream activation sequence (UAS) recognized by the DNA binding domain of the yeast HAP1 transcription activator and a basal promoter. Transgenic plants are identified and designated first corn plant lines.

In a separate transformation, corn cells are transformed with a nucleic acid construct comprising the coding sequence for a chimeric transcription activator having a DNA binding domain derived from a yeast HAP1 gene and a transcription activation domain derived from VP16. The coding sequence is operably linked to a p326 promoter (SEQ ID NO: 76). Transgenic plants are identified and designated as second corn plant lines.

Transgenic T₁ plants from each transformation are allowed to self-pollinate. Transgenic T₂ plants of each transformation are grown and pollen from second corn plant lines is used to pollinate first corn plant lines. F₁ progeny seeds are grown and are allowed to self- and/or sib-pollinate. Vegetative tissue from F₂ plants is analyzed for the amounts of campesterol, stigmasterol, β-sitosterol and α-tocopherol. Inflorescences on pollinated F₂ plants are analyzed for the amounts of the same compounds.

The experiment is repeated using first corn plant lines that contain, in addition to the second construct of Example 1, an exogenous nucleic acid construct comprising a squalene synthase coding sequence operably linked to 4 copies of the yeast HAP1 UAS. T₁, T₂, and F₁ plants are made as described above. Vegetative tissue from F₂ plants is analyzed for the amounts of campesterol, stigmasterol, β-sitosterol and α-tocopherol. Inflorescences on pollinated F₂ plants are analyzed for the amounts of the same compounds.

### Example 6

### Analysis of Transgenic California Poppy Callus Containing a Rice Methyltransferase RNAi Construct

California poppy (Eschscholzia californica) was transformed with the second RNAi construct vector of Example 1 (SEQ ID NO: 106) essentially following the procedures published by Park and Facchini, (Plant Cell Rep 19: 421-426, 1999) and (Plant Cell Rep 19: 1006-1012, 2000).

Tissues were collected from transformed callus from two independent transformation events for analysis of transcription levels of berberine bridge enzyme (BBE), N-methyl-coclaurine-hydroxylase (NMCH3), Phantastica-like Myb gene (PHAN), NADPH:ferrihemoprotein oxidoreductase (NADPH), and RuBis CO large subunit (RUBISCO). Transcription levels were analyzed in each callus sample using quantitative RT-PCR (qRT-PCR) as described below. Transcription levels of BBE, NMCH3, PHAN, NADPH and RUBISCO were also analyzed in wild-type non-transgenic California poppy callus. Transcription levels of these five genes were then normalized to the transcription levels of a tubulin gene in the same sample. The expression level of tubulin appeared to be similar in all wild-type and transgenic samples analyzed.

Total RNA was isolated from the tissue samples using Trizol Reagent (Invitrogen). RNA was converted to cDNA using the reagents included in the iScript kit (BioRad). Quantitative RT-PCR was performed using BioRad icycler reagents and an iCycler PCR machine. After determining the amount of gene transcription relative to tubulin transcription for each sample, the ratio of transcription relative to non-transgenic wild-type data was calculated. The results are shown in Table 4.

TABLE 4

| | | Ratio of Transcription in Transgenic Callus | | | | |
|---|---|---|---|---|---|---|
| | | Relative to Non-Transgenic Callus | | | | |
| Construct | | BBE | NMCH3 | PHAN | NADPH | RUBISCO |
| Os- | Event | 85.04 | 1.41 | 3.84 | 8.40 | 11.88 |
| MET1- | 1 | | | | | |
| RNAi | | | | | | |
| | Event | 207.94 | 0.21 | 0.09 | 0.44 | 2.97 |
| | 2 | | | | | |

As shown in Table 4, transcription of the BBE gene increased about 80 to 200-fold in callus containing the RNAi construct relative to BBE transcription in wild-type callus. In contrast, transcription of NMCH3, PHAN, NADPH and RUBISCO was never more than about 12-fold in transgenic callus relative to transcription of the corresponding gene in non-transgenic callus, and in some cases transcription was decreased in transgenic callus relative to non-transgenic callus.

### Example 7

### Chemical Analysis of Alkaloid Compounds in Transgenic California Poppy Callus

California poppy was transformed with the second Met 1 RNAi construct vector of Example 1 (SEQ ID NO: 106), and callus from three independent transformation events were freeze-dried. The three events were designated OsMet1-RNAi-01, OsMet1-RNAi-02 and OsMet1-RNAi-03. Twenty (20) mg of freeze-dried callus from each event was extracted in methanol using a sonicator for 4 hours. Reserpine was included during extraction to serve as an internal standard. The crude extract was then clarified using a syringe filter. Freeze-dried callus from two non-transgenic California poppy callus lines were extracted in the same manner as controls, and were designated Ec-WT-01 and Ec-WT-02.

Liquid chromatography-mass spectrometry (LC-MS) analysis was carried out on the clarified extracts, using a Waters-Micromass ZMD (single quadrupole, benchtop MS detector with positive electrospray ionization). The LC-MS conditions were a gradient of 20% to 95% acetonitrile (in 0.1% formic acid) for 55 min followed by a 5 min isocratic run in 95% acetonitrile (in 0.1% formic acid) using Alltima C18 column (5 um; 150×4.6 mm). The area of signature peaks from LC-MS data for known alkaloid intermediates was normalized to the internal standard. The results were then expressed as the ratio of the amount of the indicated alkaloid compound relative to the average amount in non-transgenic callus as determined from previous experiments. The results are shown in Table 5.

TABLE 5

| | Ratio of Amount in Transgenic Callus Relative to Average Amount in Non-Transgenic Callus | | | | | | |
|---|---|---|---|---|---|---|---|
| | San- | | | | | | |
| | guin- | | | | | | |
| Event | a- | Dihydroxy- | 12-hydroxy- | 10-hydroxy- | | | |
| # | rine | dihydrosanguinarine | dihydrochelirubine | dihydrosanguinarine | Dihydromacarpine | Dihydrochelirubine | Dihydrosanguinarine |
| Ec- | 0.99 | 1.84 | 1.78 | 1.20 | 0.51 | 1.04 | 1.03 |
| WT-01 | | | | | | | |
| Ec- | 1.01 | 0.16 | 0.22 | 0.80 | 1.49 | 0.96 | 0.97 |
| WT-03 | | | | | | | |
| OsMet1- | 1.62 | 26.04 | 14.43 | 84.57 | 0.79 | 2.11 | 16.65 |
| RNAi-01 | | | | | | | |
| OsMet1- | 1.26 | 13.58 | 5.06 | 37.76 | 0.77 | 1.58 | 8.36 |
| RNAi-02 | | | | | | | |
| OsMet1- | 1.29 | 18.13 | 6.16 | 24.61 | 1.49 | 2.43 | 6.38 |
| RNAi-03 | | | | | | | |

As shown in Table 5, the amount of each alkaloid in non-transgenic callus was similar or identical to the average amount determined in previous experiments. In contrast, the amounts of dihydroxy-dihydrosanguinarine, 12-hydroxy-dihydrochelirubine, 10-hydroxy-dihydrosanguinarine and dihydrosanguinarine increased in transgenic callus relative to the average amount in previous experiments. The extent of the increase for these four alkaloids varied from about 5-fold to more than 80-fold, depending on the compound and the particular transgenic event. The amounts of sanguinarine and dihydrochelirubine appeared to be increased slightly in transgenic callus, the extent of the increase varying from about 1.2-fold to about 2.4-fold. The amount of dihydromacarpine in transgenic callus relative to average amounts in non-transgenic callus appeared to depend on the particular event, increasing in one event but decreasing in the other two events.

### Example 8

### Analysis of Opium Poppy Callus Containing a Rice Methyltransferase RNAi Construct

Opium poppy was transformed with the second Met 1 RNAi construct vector of Example 1 (SEQ ID NO: 106) following the procedures as described below.

Explant Preparation and Embryogenic Callus Induction

Seeds of Papaver somniferum cv. Bea's Choice (Source: The Basement Shaman, Woodstock, Ill.) were surface-sterilized in 20% Clorox (commercial bleach) plus 0.1% Liqui-Nox (surfactant) for 20 min and rinsed 3 times with sterile MilliQH2O. Seeds were allowed to germinate in Germination Medium (GM; ½ strength of MS basal salts supplemented with B5 vitamins, 1.5% sucrose and 4 g/l Phytagar, pH 5.7) in Magenta boxes by incubating in Percival growth chamber with 16 hr/8 hr light/dark photo period at 25° C.

Hypocotyls, roots, and young leaves of 10 to 20 day old seedlings were cut and placed on Callus Induction Medium (CIM; MS basal medium with B5 vitamins, 1 g/1 Casamino acid, 2 mg/l 2,4 D, 0.5 mg/l BA, and 6.5 g/l Phytagar) and incubated at low light at 25° C. in Percival growth chamber. Callus was initiated from the cut surface of the explants within 20 days. Callus was subcultured onto fresh CIM. Thereafter, subculture was done every 10 to 15 days. After 2-3 subcultures compact light yellow to white spherical embryogenic callus (EmC) usually emerged from the surface of translucent friable non-embryogenic callus (NEC). EmC was separated from NEC and subcultured in CIM every 10 to 12 days.

Transformation

Agrobacterium containing the second Met1 RNAi construct vector described in Example 1 (SEQ ID NO: 106) was inoculated into 2 ml of YEB liquid medium with appropriate antibiotics and incubated overnight at 28° C. with appropriate shaking. Agrobacterium cells were spun down at 10,000 rpm in 1.5 ml eppendorf tube at room temperature (RT) using a micro-centrifuge. Cells were resuspended in 6 mls of liquid co-cultivation medium (liquid CCM=CM with 1100M acetosyringone) in 50 ml conical tube to get a final OD₆₀₀ of 0.06-0.08.

Approximately 0.5 to 1 gram of EmC was infected with the Agrobacterium suspension for 5 min with gentle agitation. Transfected EmC was blotted-dry with sterile Kimwipe paper in a Petri plate before transfer on top of sterile Whatman filter paper contained in co-cultivation Medium (CCM). Transfected EmC was incubated at 22° C. under low light in Percival growth chamber for 3 days for co-cultivation.

Transfected EmC were washed 3 times with 20-30 ml of sterile MilliQ-H₂O with moderate shaking. The last wash was done in the presence of 500 mg/l Carbenicillin. Washed EmC was briefly dried in sterile Kimwipe® paper prior to transfer in Recovery Medium (RRM=CIM+500 mg/L carbenicillin). Transfected EmC was incubated at 25° C. under low light in Percival growth chamber for 7-9 days.

After the recovery period, all calli were transferred to Callus Selection Medium (CSM=CM+500 mg/L carbenicillin+5 mg/L bialaphos) and incubated at 25° C. under low light in Percival growth chamber. Subculture of transfected EmC was done every 10 to 12 days. After the second subculture, only bialaphos resistant calli were transferred to fresh CSM. The resistant embryogenic calli typically had light yellow color. Non-resistant calli typically were light to dark brown in color and were dead or dying.

After 3 subcultures, bialaphos resistant calli were transferred to Regeneration Medium 1 (RM1=CM+250 mg/L carbenicillin+2 mg/l Zeatin+0.05 mg/l IBA+100 mg/l L-Glutamine+200 mg/l L-Cysteine) and incubated at 25° C. under high light in a Percival growth chamber with a 16 hr photo period.

After 10-15 days, bialaphos resistant calli were transferred to Regeneration Medium 2 (RM2=CM+250 mg/L carbenicillin+0.5 mg/l Zeatin+0.05 mg/l IBA+100 mg/l L-Glutamine+200 mg/l L-Cysteine). Bialaphos resistant EmC continue to grow and differentiate into embryos. These embryos developed into plantlets after 15-20 days.

Small plantlets with roots were transferred to Rooting Medium (RtM=CM+250 mg/L carbenicillin+0.2 mg/l IBA+50 mg/l L-Glutamine+4 g/l Phytagar) in a sterile Sundae Cup. Fully-regenerated plants are transferred to soil at appropriate time.

Protocol for qRT-PCR Analysis

Embryogenic callus tissues from nine independent transgenic events were collected and used for qRT-PCR analysis. Similar tissue was also collected from wild-type lines as a control. Total RNA was isolated from the tissue samples using Trizol Reagent (Invitrogen). RNA was converted to cDNA using the reagents included in the iScript kit (BioRad). Quantitative RT-PCR was performed using BioRad iCycler reagents and iCycler PCR machine.

Opium poppy CAB (chlorophyll-a/b binding protein) gene was used to normalize the expression of different alkaloid-related genes in the samples. Transcription of the genes listed below was monitored for each of the transgenic events using a set of primers specific for each gene.

TABLE 6

| Gene | |
|---|---|
| Abbreviation | Enzyme Name |
| CR | Codeinone reductase (EC 1.1.1.247) |
| BBE | Berberine bridge enzyme (EC 1.21.3.3) |
| HMCOMT1 | S-adenosyl-L-methionine: 3′-hydroxy-N- |
| | methylcoclaurine 4′-O-methyltransferase 1 (EC 2.1.1.116) |
| HMCOMT2 | S-adenosyl-L-methionine: 3′-hydroxy-N- |
| | methylcoclaurine 4′-O-methyltransferase 2 (EC 2.1.1.116) |
| NOMT | (R,S)-norcoclaurine 6-O-methyltransferase (EC 2.1.1.128) |
| ROMT | (R,S)-reticuline 7-O-methyltransferase |
| SAT | Salutaridinol 7-O-acetyltransferase (EC 2.3.1.150) |
| YDC | Tyrosine decarboxylase (EC 4.1.1.25) |
| (or TYD) | |

Summary of the qRT-PCR Results:

The values for each gene shown below are the ratio of transcription for each of the transformation events relative to the averaged value of non-transgenic wild-type after normalization to CAB gene expression.

TABLE 7

| | Ratio of Transcription in Transgenic Callus | | | | | | | |
|---|---|---|---|---|---|---|---|---|
| | Relative to Non-Transgenic Callus | | | | | | | |
| | PsYDC | PsBBE | PsSAT | PsHMCOMT1 | PsHMCOMT2 | PsCR | PsNOMT | PsROMT |
| Event 1 | 1.58 | 0.57 | 0.81 | 0.47 | 3.64 | 36.75 | 0.53 | 1.14 |
| Event 10 | 2.46 | 0.43 | 0.52 | 0.61 | 0.7 | 2.57 | 0.24 | 1.04 |
| Event 12 | 2.89 | 0.69 | 1.31 | 0.38 | 3.4 | 35.09 | 1.17 | 0.2 |
| Event 2 | 3.4 | 0.97 | 1.7 | 0.83 | 5.4 | 97.005 | 6.06 | 1.02 |
| Event 3 | 3.17 | 0.75 | 1 | 0.62 | 2.96 | 29.85 | 1.66 | 0.69 |
| Event 4 | 1.41 | 0.39 | 0.38 | 0.43 | 0.42 | 1.44 | 0.044 | 0.91 |
| Event 5 | 0.21 | 0.3 | 1.12 | 0.23 | 3.03 | 12.12 | 0.65 | 0.83 |
| Event 8 | 2.63 | 0.7 | 0.79 | 0.72 | 0.97 | 4.48 | 0.29 | 1.28 |
| Event 9 | 2.96 | 0.57 | 0.89 | 0.74 | 1.62 | 7.29 | 0.67 | 1.17 |

As shown in Table 7, the CR gene was shown to be transcriptionally activated between 2.5 to 97-fold in eight of the nine transformation events tested. The HMCOMT2 gene was transcriptionally activated between 3 to 5-fold in five transformation events.

### Example 9

### Transcription of Biosynthetic Genes in Opium Poppy Plants Containing an Arabidopsis Methyltransferase RNAi Construct

An Arabidopsis Methyltransferase RNAi (AtMET1 RNAi) construct was made by operably linking a CaMV35S promoter to about 2.7 kb of the Arabidopsis Met1 sequence in sense orientation and an inverted repeat of a nos terminator sequence. See, US Patent Publication 2005/0081261. The construct was made using standard molecular biology techniques, and inserted into a vector that contained a selectable marker gene conferring resistance to the herbicide Basta®.

Opium poppy tissue was transformed with the vector containing the AtMET1 RNAi construct according to the protocol described in Example 8. Plants were regenerated from several independent transformation events.

After growing for ten weeks in a greenhouse, leaf tissue was collected from plants of each event and used for qRT-PCR analysis. As controls, similar tissue was collected from wild-type lines and from transgenic lines expressing a YP188::Green Fluorescent Protein construct. The opium poppy Elongation Factor 1-β gene was used to normalize the expression of different alkaloid-related genes in the samples. Transcription of the genes listed below was monitored for each of the transgenic events as described in Example 8. The results of the qRT-PCR analysis are shown in Table 8.

As shown in Table 8, transcription of opium poppy tyrosine decarboxylase (PsYDC) was significantly higher in several transgenic events containing the AtMET1 RNAi construct, relative to non-transgenic regenerated wild-type plants. Control opium poppy lines transgenic for a YP188::GFP construct did not exhibit a significant increase in transcription from any of the biosynthetic genes tested.

TABLE 8

| | Ratio of Transcription in Transgenic Leaf Tissue Relative to Non-Transgenic Leaf Tissue | | | | | | | |
|---|---|---|---|---|---|---|---|---|
| | PsBBE | PsCR | PsSAT | PsHMCOMT1 | PsHMCOMT2 | PsROMT | PsNOMT | PsYDC |
| PsGFP 6-03 | 0.27 | 0.56 | 0.49 | 0.18 | 0.91 | 0.30 | 0.52 | 0.68 |
| PsGFP 4-01 | 0.47 | 1.09 | 0.27 | 0.47 | 0.51 | 0.32 | 0.40 | 0.76 |
| PsEvent 1-11 | 0.50 | 0.64 | 0.49 | 0.49 | 0.52 | 0.28 | 0.42 | 52.59 |
| PsEvent 2-05 | 1.62 | 1.41 | 0.89 | 1.18 | 1.48 | 1.39 | 1.77 | 1.40 |
| PsEvent 2-04 | 0.63 | 1.37 | 0.35 | 0.96 | 0.78 | 0.36 | 0.72 | 1.20 |
| PsEvent 2-03 | 0.11 | 0.93 | 0.40 | 0.38 | 0.46 | 0.32 | 0.42 | 3.52 |
| PsEvent 2-01 | 0.91 | 0.79 | 0.81 | 1.03 | 1.38 | 0.76 | 0.79 | 7.64 |
| PsEvent 1-05 | 0.29 | 0.45 | 0.95 | 0.63 | 0.79 | 0.56 | 0.55 | 1.33 |
| PsEvent 1-04 | 0.59 | 0.30 | 0.14 | 0.02 | 0.22 | 0.05 | 0.17 | 74.37 |
| PsEvent 1-03 | 0.56 | 0.39 | 0.99 | 0.59 | 0.93 | 0.53 | 0.77 | 42.71 |

### Example 10

### Level of AtMET1 RNAi Transcript in Opium Poppy Plants

The opium poppy leaf tissue of Example 9 was also analysed by qRT-PCR for the level of AtMET1 RNAi transcription. The results are shown in Table 9.

TABLE 9
Transcription of AtMET1 RNAi*

| | AtMET1 |
|---|---|
| PsGFP 6-03 | 2.09 |
| PsGFP 4-01 | 0.55 |
| PsEvent 1-11 | 1516.64 |
| PsEvent 2-05 | 67067.82 |
| PsEvent 2-04 | 116771.85 |
| PsEvent 2-03 | 29875.32 |
| PsEvent 2-01 | 337.79 |
| PsEvent 1-05 | 16009.79 |
| PsEvent 1-04 | 11320.63 |
| PsEvent 1-03 | 7822.06 |

*Amount of AtMET1 transcript relative to non-transgenic control.

### Example 11

### Determination of Ortholog/Functional Homolog Sequences

A subject sequence was considered a functional homolog and/or ortholog of a query sequence if the subject and query sequences encode proteins having a similar function and/or activity. A process known as Reciprocal BLAST (Rivera et al, Proc. Natl. Acad. Sci. USA, 1998, 95:6239-6244) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides were designated as a cluster.

The main Reciprocal BLAST process consists of two rounds of BLAST searches; forward search and reverse search. In the forward search step, a query polypeptide sequence, “polypeptide A,” from source species S^{A} was BLASTed against all protein sequences from a species of interest. Top hits were determined using an E-value cutoff of 10⁻⁵ and an identity cutoff of 35%. Among the top hits, the sequence having the lowest E-value was designated as the best hit, and considered a potential functional homolog and/or ortholog. Any other top hit that had a sequence identity of 80% or greater to the best hit or to the original query polypeptide was considered a potential functional homolog and/or ortholog as well. This process was repeated for all species of interest.

In the reverse search round, the top hits identified in the forward search from all species were BLASTed against all protein sequences from the source species S^{A}. A top hit from the forward search that returned a polypeptide from the aforementioned cluster as its best hit was also considered as a potential functional homolog and/or ortholog.

Functional homologs and/or orthologs were identified by manual inspection of potential functional homolog and/or ortholog sequences. Representative functional homologs and/or orthologs for SEQ ID NOS: 121, 135 and 109 are shown in FIGS. 1 through 3, respectively.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of producing a secondary metabolite, said method comprising:
a) extracting at least one secondary metabolite from plant cells transformed with a recombinant nucleic acid construct, said construct comprising a regulatory region operably linked to a nucleic acid that decreases expression of a methylation status polypeptide.

2. The method of claim 1, wherein said secondary metabolite is a terpenoid compound.

3. The method of claim 2, wherein said secondary metabolite is a sterol compound.

4. The method of claim 2, wherein said terpenoid compound is selected from the group consisting of geranyl diphosphate, linalyl acetate, carvone, nerol, menthol, β-ocimene, pinene, limonene, 1,8 cineole, myrcene, (+)-bornyl diphosphate, (−) isopiperitenone, (+)-pulegone, (−)-menthone, thujone, marinol, tetrahydrocannabinol, camphor, borneol, perillyl alcohol, thymol, sobrerol, sabinene, farnesyl diphosphate, E-β-farnesene, β-caryophyllene, 5-epi-aristolochene, vetispiradiene, δ-cadinene, gerrnacrene C, E-α-bisabolene, δ-selinene, parthenolide, artemisinin, artemisin, artemether, santonin, parthenolide, gossypol, manoalide, acetyldigoxin, digoxin, deslanoside, digitalin, digitoxin, lanatosides A, B and C, γ-humulene, geranylgeranyl diphosphate, ent-copalyl diphosphate, ent-kaurene, taxadiene, taxol, baccatin III, calanolide A, ginkgolides, casbene, abietadiene, andrographolide, neoandrographolide, forskolin, resiniferatoxin, pseudopterosin C, methopterosin, carnosic acid, carnosol, tanshinone II-A, saprorthoquinone, triptolide, cambrene, squalene, lupeol, α-amyrin, β-amyrin, glycyrrhizin, β-sitosterol, sitostanol, stigrnasterol, campesterol, ergosterol, diosgenin, aescin, picrotoxin, betulinic acid, asiaticoside, cucurbitacin E, glycyrrhizin, diosgenin, ruscogenin, lycopene, β-carotene, zeta-carotene, lutein, zeaxanthin, antheraxanthin, phytoene, bixin, astaxanthin, yuanhuacin, yuanhuadin, glaucarubin, convallatoxin, squalamine, ouabain, and strophanthidin.

5. The method of claim 1, wherein said secondary metabolite is an alkaloid compound.

6. The method of claim 5, wherein said alkaloid compound is selected from the group consisting of salutaridine, salutaridinol, salutaridinol acetate, thebaine, isothebaine, oripavine, morphinone, morphine, codeine, codeinone, papaverine, narcotine, narceine, hydrastine, and neopinone.

7. The method of claim 5, wherein said alkaloid compound is selected from the group consisting of berberine, palmatine, tetrahydropalmatine, S-canadine, columbamine, S-tetrahydrocolumbamine, S-scoulerine, S-cheilathifoline, S-stylopine, S-cis-N-methylstylopine, protopine, 6-hydroxyprotopine, R-norreticuline, S-norreticuline, R-reticuline, S-reticuline, 1,2-dehydroreticuline, S-3′-hydroxycoclaurine, S-norcoclaurine, S-coclaurine, S—N-methylcoclaurine, berbamunine, 2′-norberbamunine, laudanosine, nororientaline and guatteguamerine.

8. The method of claim 5, wherein said alkaloid compound is selected from the group consisting of: sanguinarine, dihydrosanguinarine, dihydroxy-dihydrosanguinarine, 12-hydroxy-dihydrochelirubine, 10-hydroxy-dihydrosanguinarine, dihydromacarpine, dihydrochelirubine, dihydrosanguinarine, chelirubine, 12-hydroxy-chelirubine, dihydromacarpine, and macarpine.

9. The method of claim 1, wherein said methylation status polypeptide is a cytosine DNA methyltransferase, or a decreased DNA methylation polypeptide.

10. The method of claim 1, wherein said cells are monocotyledonous cells.

11. The method of claim 10, wherein said monocotyledonous cells are part of a whole plant.

12. The method of claim 11, wherein said regulatory region confers transcription in photosynthetically active tissue.

13. The method of claim 1, wherein said cells are dicotyledonous cells.

14. The method of claim 13, wherein said dicotyledonous cells are part of a whole plant.

15. The method of claim 14, wherein said whole plant is a Papaveraceae plant.

16. The method of claim 15, wherein said regulatory region confers transcription in laticifer cells, companion cells or sieve cells.

17. A method of producing a secondary metabolite, said method comprising:
a) growing plant cells comprising a recombinant nucleic acid construct, said construct comprising a regulatory region operably linked to a nucleic acid that decreases expression of a methylation status polypeptide, wherein expression of said nucleic acid is effective for modulating the amount of at least one secondary metabolite in said cell.

18. The method of claim 12, wherein said secondary metabolite is a terpenoid compound.

19. The method of claim 12, wherein said secondary metabolite is an alkaloid compound.

20. The method of claim 12, wherein said methylation status polypeptide is a cytosine DNA methyltransferase, or a decrease in DNA methylation polypeptide.

21. The method of claim 12, further comprising the step of extracting said secondary metabolite from said plant cells.

22. The method of claim 12, wherein said cells are part of a whole plant.

23. The method of claim 22, wherein said plant is an alkaloid producing plant.

24. The method of claim 22, wherein said plant is a terpenoid producing plant.

25. A plant comprising a recombinant nucleic acid construct, said construct comprising a nucleic acid that decreases expression of a methylation status polypeptide operably linked to a regulatory region, wherein expression of said nucleic acid is effective for modulating the amount of at least one secondary metabolite in a tissue of said plant relative to the amount in the corresponding tissue of a control plant that lacks said recombinant nucleic acid construct.

26. The plant of claim 25, wherein said plant is a monocotyledonous plant.

27. The plant of claim 21, wherein said plant is a dicotyledonous plant.

28. The plant of claim 21, wherein said secondary metabolite is a terpenoid compound, an alkaloid compound, or a phenylpropanoid compound.

29. The plant of claim 21, wherein said methylation status polypeptide is a cytosine DNA methyltransferase, or a decrease in DNA methylation polypeptide.

30. A plant comprising a first recombinant nucleic acid construct, said first construct comprising a first transcription activator recognition site operably linked to a nucleic acid that decreases expression of a methylation status polypeptide, and a first exogenous activator nucleic acid encoding a first transcription activator operably linked to a first promoter, wherein said first transcription activator is effective for binding to said first recognition site.

31. The plant of claim 30, wherein said first promoter is a broadly expressing promoter and said plant is male-sterile.

32. The plant of claim 30, further comprising a second recombinant nucleic acid construct, said second construct comprising a second transcription activator recognition site operably linked to a coding sequence for an endogenous gene involved in secondary metabolite biosynthesis.

33. The plant of claim 32, wherein said first transcription activator is effective for binding to said second recognition site.

34. The plant of claim 32, further comprising a second exogenous activator nucleic acid encoding a second transcription activator operably linked to a second promoter, wherein said second transcription activator is effective for binding to said second recognition site.

35. The plant of claim 34, wherein said first promoter is a broadly expressing promoter and second promoter is a maturing endosperm promoter.

36. The plant of claim 34, wherein said first promoter is a maturing endosperm promoter and said second promoter is a maturing endosperm promoter.
